SEQUENCE LISTING (8 sequences)

SEQ ID NO: 1 - DNA, 24 bp, synthetic construct
  source 1..24: mol_type = other DNA
gaacagcactggtggtcagacacc

SEQ ID NO: 2 - DNA, 20 bp, synthetic construct
  source 1..20: mol_type = other DNA
tggcaccagtgtccggatta

SEQ ID NO: 3 - DNA, 22 bp, synthetic construct
  source 1..22: mol_type = other DNA
atggatgcctctgctctcactg

SEQ ID NO: 4 - DNA, 22 bp, synthetic construct
  source 1..22: mol_type = other DNA
cccgatgagaatggcagaaagc

SEQ ID NO: 5 - DNA, 23 bp, synthetic construct
  source 1..23: mol_type = other DNA
ctctacacagtcacgggacaaag

SEQ ID NO: 6 - DNA, 22 bp, synthetic construct
  source 1..22: mol_type = other DNA
ctgtggtgctttgaggtaggtc

SEQ ID NO: 7 - DNA, 24 bp, synthetic construct
  source 1..24: mol_type = other DNA
ttcgacagtcagccgcatcttctt

SEQ ID NO: 8 - DNA, 23 bp, synthetic construct
  source 1..23: mol_type = other DNA
caggcgcccaatacgaccaaatc

(19) United States
(12) Patent Application Publication
(10) Pub. No.: US 2026/0364061 A1
(43) Pub. Date: Nov. 19, 2026

(54) COMPOUNDS, COMPOSITIONS, AND METHODS FOR THE TREATMENT OF CANCER

(71) Applicant: Ohio State Innovation Foundation, Columbus, OH (US)

(72) Inventors: Joal BEANE, Columbus, OH (US); Marina GORYUNOVA, Columbus, OH (US); Yiran HE, San Diego, CA (US); Christopher HADAD, Dublin, OH (US); Hua ZHU, Columbus, OH (US)

(21) Appl. No.: 19/487,317

(22) PCT Filed: May 23, 2024
(86) PCT No.: PCT/US2024/030880

§ 371 Date: Nov. 24, 2025

Related U.S. Application Data
(60) Provisional application No. 63/468,515, filed on May 23, 2023.

(51) Int. Cl. A61K 31/4535; A61K 31/445; A61K 31/454; A61P 35/00

(52) U.S. Cl. CPC A61K 31/4535; A61K 31/445; A61K 31/454; A61P 35/00

(57) ABSTRACT

Provided herein are compounds, compositions, and methods for the treatment of cancer, in particular lipocarcinomas (e.g., dedifferentiated liposarcomas).

### CROSS-REFERENCE TO RELATED APPLICATIONS

This application claims benefit of U.S. Provisional Application No. 63/468,515, filed May 23, 2023, which is hereby incorporated herein by reference in its entirety.

### REFERENCE TO SEQUENCE LISTING

The sequence listing submitted May 23, 2024, as an .XML file entitled “103361-517WO1_ST26” created on May 23, 2024, and having a file size of 8,091 bytes is hereby incorporated by reference pursuant to 37 C.F.R. § 1.52(e)(5).

### BACKGROUND

Current therapies for treating many cancers have limited efficacy. For example, surgery remains the only curative treatment for patients with liposarcoma (LPS). The translation of novel therapies has been hampered, and there has been no significant improvement in prognosis for 30 years. Accordingly, there remains a need for improved methods for the treatment of cancer.

### SUMMARY

Provided herein are methods of treating cancer in a subject (e.g., a human subject) in need thereof. These methods can comprise comprising administering to the subject an effective amount of an IWS1 inhibitor to treat the cancer.

In some embodiments, the cancer comprises a solid tumor.

In some embodiments, cancer comprises a soft tissue sarcoma.

In certain embodiments, the cancer is characterized by elevated levels of IWS1.

In certain embodiments, the cancer comprises a liposarcoma (LPS), such as dedifferentiated LPS (ddLPS).

In some embodiments, the IWS1 inhibitor is administered in combination with an additional treatment for the cancer (e.g., radiotherapy, surgery, administration of one or more additional active agents, or a combination thereof).

In some embodiments, the IWS1 inhibitor reduces the migration ability of cancer cells in the subject, reduces the invasiveness of cancer cells in the subject, reduces metastasis of the cancer, or a combination thereof.

Also provided herein are methods for preventing metastisis of a liposarcoma in a subject or reducing the rate of metastisis of a liposarcoma. These methods can comprise administering to the subject an effective amount of an IWS1 inhibitor to prevent metastisis of the liposarcoma or reduce the rate of metastisis of the liposarcoma. In certain embodiments, the liposarcoma can comprise dedifferentiated LPS (ddLPS).

Also provided herein are methods for reducing the migration of liposarcoma cells in a subject. These methods can comprise administering to the subject an effective amount of an IWS1 inhibitor to reduce the migration of the liposarcoma cells. In certain embodiments, the liposarcoma can comprise dedifferentiated LPS (ddLPS).

Also provided herein are methods for reducing the invasiveness of liposarcoma cells in a subject. These methods can comprise administering to the subject an effective amount of an IWS1 inhibitor to reduce the invasiveness of the liposarcoma cells. In certain embodiments, the liposarcoma can comprise dedifferentiated LPS (ddLPS).

Any suitable IWS1 inhibitor can be used in connection with the methods described herein. In some embodiments, the IWS1 inhibitor can target an IWS1/Spt6 interface. In some embodiments, the IWS1 inhibitor can reduce the binding of IWS1 and Spt6. In some embodiments, the IWS1 inhibitor reduces the formation of the RNAPII complex. In some embodiments, the IWS1 inhibitor down-regulates the transcriptional activities of RNAPII.

In certain embodiments, the IWS1 inhibitor comprises a tricyclic fused ring system.

In some embodiments, the IWS1 inhibitor is defined by Formula I or Formula II

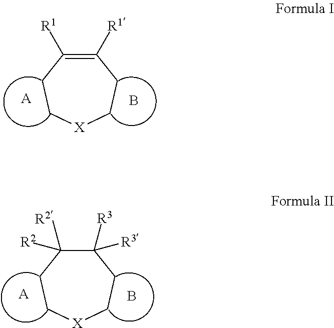

or a pharmaceutically acceptable salt or prodrug thereof, wherein
   X is NR⁴, CR⁵R^{5′}, or —C═CR⁶R^{6′};
   A and B are each individually selected from a fused 3-10 membered monocyclic or bicyclic cycloalkyl ring, a fused 3-10 membered monocyclic or bicyclic hecycloalkyl ring, a fused 5-11 membered monocyclic or bicyclic aryl ring, and a fused 5-11 membered monocyclic or bicyclic heteroaryl ring, each optionally substituted by 1, 2, 3, or 4 independently selected R^{A} groups;
   R¹ and R^{1′} are individually selected from H, halo, OH, OR^{B}, CN, NO₂, C₁₋₄ alkyl, C₁₋₄ haloalkyl, C₂₋₄ alkenyl, or C₂₋₄ alkynyl, wherein the C₁₋₄ alkyl, C₁₋₄ haloalkyl, C₂₋₄ alkenyl, and C₂₋₄ alkynyl groups are each optionally substituted by 1, 2, 3, or 4 independently selected R^{A} groups;
   R² and R^{2′} are individually selected from H, halo, OH, OR^{B}, CN, NO₂, C₁₋₄ alkyl, C₁₋₄ haloalkyl, C₂₋₄ alkenyl, or C₂₋₄ alkynyl, wherein the C₁₋₄ alkyl, C₁₋₄ haloalkyl, C₂₋₄ alkenyl, and C₂₋₄ alkynyl groups are each optionally substituted by 1, 2, 3, or 4 independently selected R^{A} groups; or wherein R² and R^{2′} together form a ketone group;
   R³ and R^{3′} are individually selected from H, halo, OH, OR^{B}, CN, NO₂, C₁₋₄ alkyl, C₁₋₄ haloalkyl, C₂₋₄ alkenyl, or C₂₋₄ alkynyl, wherein the C₁₋₄ alkyl, C₁₋₄ haloalkyl, C₂₋₄ alkenyl, and C₂₋₄ alkynyl groups are each optionally substituted by 1, 2, 3, or 4 independently selected R^{A} groups; or wherein R³ and R^{3′} together form a ketone group;
   R⁴ is selected from hydrogen, C₁₋₆ alkyl, C₂₋₆ alkenyl, C₂₋₆ alkynyl, C₁₋₄ haloalkyl, C₁₋₆ alkoxy, C₁₋₆ haloalkoxy, a 3-10 membered monocyclic or bicyclic cycloalkyl ring, a 3-10 membered monocyclic or bicyclic hecycloalkyl ring, a 5-11 membered monocyclic or bicyclic aryl ring, a 5-11 membered monocyclic or bicyclic heteroaryl ring, cyano-C₁₋₃ alkyl, HO—C₁₋₃ alkyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino, each optionally substituted by 1, 2, 3, or 4 independently selected R^{A} groups;
   R⁵ and R^{5′} are individually selected from H, halo, OH, OR^{B}, CN, NO₂, C₁₋₆ alkyl, C₂₋₆ alkenyl, C₂₋₆ alkynyl, C₁₋₄ haloalkyl, C₁₋₆ alkoxy, C₁₋₆ haloalkoxy, a 3-10 membered monocyclic or bicyclic cycloalkyl ring, a 3-10 membered monocyclic or bicyclic hecycloalkyl ring, a 5-11 membered monocyclic or bicyclic aryl ring, a 5-11 membered monocyclic or bicyclic heteroaryl ring, each optionally substituted by 1, 2, 3, or 4 independently selected R^{A} groups; or wherein R⁵ and R^{5′} together form a ketone group;
   R⁶ and R^{6′} are individually selected from H, halo, OH, OR^{B}, CN, NO₂, C₁₋₆ alkyl, C₂₋₆ alkenyl, C₂₋₆ alkynyl, C₁₋₄ haloalkyl, C₁₋₆ alkoxy, C₁₋₆ haloalkoxy, a 3-10 membered monocyclic or bicyclic cycloalkyl ring, a 3-10 membered monocyclic or bicyclic hecycloalkyl ring, a 5-11 membered monocyclic or bicyclic aryl ring, a 5-11 membered monocyclic or bicyclic heteroaryl ring, each optionally substituted by 1, 2, 3, or 4 independently selected R^{A} groups;
   each R^{A} is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino; and
   R^{B} is selected from hydrogen, C₁₋₆ alkyl, C₁₋₄haloalkyl, C₂₋₆ alkenyl, C₂₋₆ alkynyl, C₃₋₁₀ cycloalkyl, and 6-10 membered aryl, wherein the C₁₋₆ alkyl, C₁₋₄haloalkyl, C₂₋₆ alkenyl, C₂₋₆ alkynyl, C₃₋₁₀ cycloalkyl, and 6-10 membered aryl are each optionally substituted by 1, 2, 3, or 4 independently selected R^{A} groups.

In some embodiments, the IWS1 inhibitor is defined by Formula IA or Formula IIA

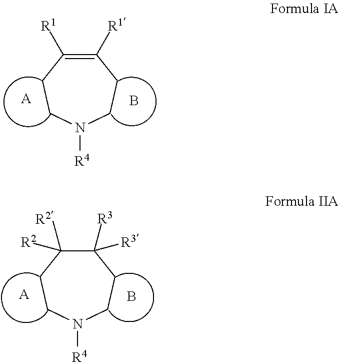

or a pharmaceutically acceptable salt or prodrug thereof, wherein
   A and B are each individually selected from a fused 3-10 membered monocyclic or bicyclic cycloalkyl ring, a fused 3-10 membered monocyclic or bicyclic hecycloalkyl ring, a fused 5-11 membered monocyclic or bicyclic aryl ring, and a fused 5-11 membered monocyclic or bicyclic heteroaryl ring, each optionally substituted by 1, 2, 3, or 4 independently selected R^{A} groups;
   R¹ and R^{1′} are individually selected from H, halo, OH, OR^{B}, CN, NO₂, C₁₋₄ alkyl, C₁₋₄ haloalkyl, C₂₋₄ alkenyl, or C₂₋₄ alkynyl, wherein the C₁₋₄ alkyl, C₁₋₄ haloalkyl, C₂₋₄ alkenyl, and C₂₋₄ alkynyl groups are each optionally substituted by 1, 2, 3, or 4 independently selected R^{A} groups;
   R² and R^{2′} are individually selected from H, halo, OH, OR^{B}, CN, NO₂, C₁₋₄ alkyl, C₁₋₄ haloalkyl, C₂₋₄ alkenyl, or C₂₋₄ alkynyl, wherein the C₁₋₄ alkyl, C₁₋₄ haloalkyl, C₂₋₄ alkenyl, and C₂₋₄ alkynyl groups are each optionally substituted by 1, 2, 3, or 4 independently selected R^{A} groups; or wherein R² and R^{2′} together form a ketone group;
   R³ and R^{3′} are individually selected from H, halo, OH, OR^{B}, CN, NO₂, C₁₋₄ alkyl, C₁₋₄ haloalkyl, C₂₋₄ alkenyl, or C₂₋₄ alkynyl, wherein the C₁₋₄ alkyl, C₁₋₄ haloalkyl, C₂₋₄ alkenyl, and C₂₋₄ alkynyl groups are each optionally substituted by 1, 2, 3, or 4 independently selected R^{A} groups; or wherein R³ and R^{3′} together form a ketone group;
   R⁴ is selected from hydrogen, C₁₋₆ alkyl, C₂₋₆ alkenyl, C₂₋₆ alkynyl, C₁₋₄ haloalkyl, C₁₋₆ alkoxy, C₁₋₆ haloalkoxy, a 3-10 membered monocyclic or bicyclic cycloalkyl ring, a 3-10 membered monocyclic or bicyclic hecycloalkyl ring, a 5-11 membered monocyclic or bicyclic aryl ring, a 5-11 membered monocyclic or bicyclic heteroaryl ring, cyano-C₁₋₃ alkyl, HO—C₁₋₃ alkyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino, each optionally substituted by 1, 2, 3, or 4 independently selected R^{A} groups;
   each R^{A} is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino; and
   R^{B} is selected from hydrogen, C₁₋₆ alkyl, C₁₋₄haloalkyl, C₂₋₆ alkenyl, C₂₋₆ alkynyl, C₃₋₁₀ cycloalkyl, and 6-10 membered aryl, wherein the C₁₋₆ alkyl, C₁₋₄haloalkyl, C₂₋₆ alkenyl, C₂₋₆ alkynyl, C₃₋₁₀ cycloalkyl, and 6-10 membered aryl are each optionally substituted by 1, 2, 3, or 4 independently selected R^{A} groups.

In some of these embodiments, R⁴ is C₁₋₆ alkyl optionally substituted by 1, 2, 3, or 4 independently selected R^{A} groups.

In some embodiments, the IWS1 inhibitor is defined by Formula IB or Formula IIB

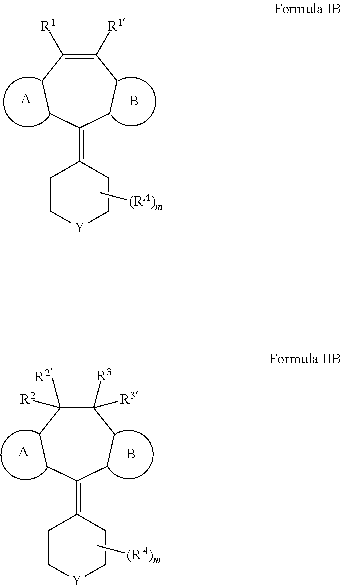

or a pharmaceutically acceptable salt or prodrug thereof, wherein
   Y is NR⁴ or CR⁵R^{5′};
   A and B are each individually selected from a fused 3-10 membered monocyclic or bicyclic cycloalkyl ring, a fused 3-10 membered monocyclic or bicyclic hecycloalkyl ring, a fused 5-11 membered monocyclic or bicyclic aryl ring, and a fused 5-11 membered monocyclic or bicyclic heteroaryl ring, each optionally substituted by 1, 2, 3, or 4 independently selected R^{A} groups;
   R¹ and R^{1′} are individually selected from H, halo, OH, OR^{B}, CN, NO₂, C₁₋₄ alkyl, C₁₋₄ haloalkyl, C₂₋₄ alkenyl, or C₂₋₄ alkynyl, wherein the C₁₋₄ alkyl, C₁₋₄ haloalkyl, C₂₋₄ alkenyl, and C₂₋₄ alkynyl groups are each optionally substituted by 1, 2, 3, or 4 independently selected R^{A} groups;
   R² and R^{2′} are individually selected from H, halo, OH, OR^{B}, CN, NO₂, C₁₋₄ alkyl, C₁₋₄ haloalkyl, C₂₋₄ alkenyl, or C₂₋₄ alkynyl, wherein the C₁₋₄ alkyl, C₁₋₄ haloalkyl, C₂₋₄ alkenyl, and C₂₋₄ alkynyl groups are each optionally substituted by 1, 2, 3, or 4 independently selected R^{A} groups; or wherein R² and R^{2′} together form a ketone group;
   R³ and R^{3′} are individually selected from H, halo, OH, OR^{B}, CN, NO₂, C₁₋₄ alkyl, C₁₋₄ haloalkyl, C₂₋₄ alkenyl, or C₂₋₄ alkynyl, wherein the C₁₋₄ alkyl, C₁₋₄ haloalkyl, C₂₋₄ alkenyl, and C₂₋₄ alkynyl groups are each optionally substituted by 1, 2, 3, or 4 independently selected R^{A} groups; or wherein R³ and R^{3′} together form a ketone group;
   R⁴ is selected from hydrogen, C₁₋₆ alkyl, C₂₋₆ alkenyl, C₂₋₆ alkynyl, C₁₋₄ haloalkyl, C₁₋₆ alkoxy, C₁₋₆ haloalkoxy, a 3-10 membered monocyclic or bicyclic cycloalkyl ring, a 3-10 membered monocyclic or bicyclic hecycloalkyl ring, a 5-11 membered monocyclic or bicyclic aryl ring, a 5-11 membered monocyclic or bicyclic heteroaryl ring, cyano-C₁₋₃ alkyl, HO—C₁₋₃ alkyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁-6 alkyl)aminocarbonylamino, each optionally substituted by 1, 2, 3, or 4 independently selected R^{A} groups;
   R⁵ and R^{5′} are individually selected from H, halo, OH, OR^{B}, CN, NO₂, C₁₋₆ alkyl, C₂₋₆ alkenyl, C₂₋₆ alkynyl, C₁₋₄ haloalkyl, C₁₋₆ alkoxy, C₁₋₆ haloalkoxy, a 3-10 membered monocyclic or bicyclic cycloalkyl ring, a 3-10 membered monocyclic or bicyclic hecycloalkyl ring, a 5-11 membered monocyclic or bicyclic aryl ring, a 5-11 membered monocyclic or bicyclic heteroaryl ring, each optionally substituted by 1, 2, 3, or 4 independently selected R^{A} groups;
   each R^{A} is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino;
   m is an integer from 0 to 4; and
   R^{B} is selected from hydrogen, C₁₋₆ alkyl, C₁₋₄haloalkyl, C₂₋₆ alkenyl, C₂₋₆ alkynyl, C₃₋₁₀ cycloalkyl, and 6-10 membered aryl, wherein the C₁₋₆ alkyl, C₁₋₄haloalkyl, C₂₋₆ alkenyl, C₂₋₆ alkynyl, C₃₋₁₀ cycloalkyl, and 6-10 membered aryl are each optionally substituted by 1, 2, 3, or 4 independently selected R^{A} groups.

In some of these embodiments, Y is NR⁴. In certain of these embodiments, R⁴ is selected from hydrogen and C₁₋₆ alkyl.

In some of these embodiments, m is 0.

In some of the embodiments above, A is a phenyl ring. In other embodiments described above, A is a pyridyl ring. In other embodiments described above, A is a 5-membered heterocyclic ring chosen from a thiophene ring, a pyrrolidine ring, a pyrroline ring, a pyrrole ring, a pyrazolidine ring, an imidazolidine ring, a pyrazoline ring, and imidazoline ring, a pyrazole ring, a imidazole ring, a triazole ring, a furyl ring, a dioxolane ring, a thiophenyl ring, an oxazole ring, an isoxazole ring, an isothiazole ring, a thiazole ring, an oxathiolane ring, oxadiazole ring, or a thiadiazole ring.

In some of the embodiments above, B is a phenyl ring. In other embodiments described above, B is a pyridyl ring. In other embodiments described above, B is a 5-membered heterocyclic ring chosen from a thiophene ring, a pyrrolidine ring, a pyrroline ring, a pyrrole ring, a pyrazolidine ring, an imidazolidine ring, a pyrazoline ring, and imidazoline ring, a pyrazole ring, a imidazole ring, a triazole ring, a furyl ring, a dioxolane ring, a thiophenyl ring, an oxazole ring, an isoxazole ring, an isothiazole ring, a thiazole ring, an oxathiolane ring, oxadiazole ring, or a thiadiazole ring.

In certain embodiments described above, A is a phenyl ring and B is a phenyl ring.

In other embodiments described above, A is a phenyl ring and B is a pyridyl ring. In other embodiments, A is a phenyl ring and B is a thiophene ring.

In some examples, the IWS1 inhibitor comprises a compound shown below

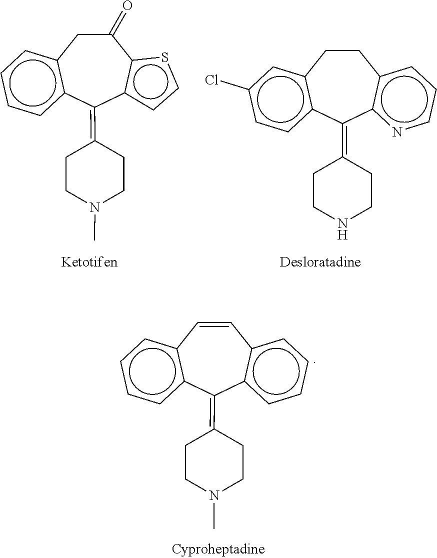

In some examples, the IWS1 inhibitor comprises a compound shown below

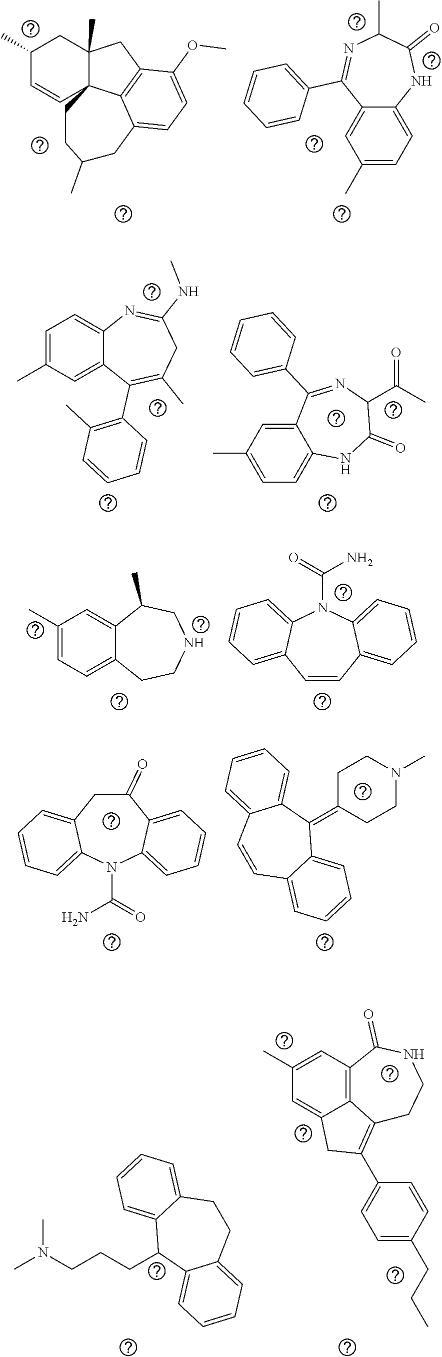

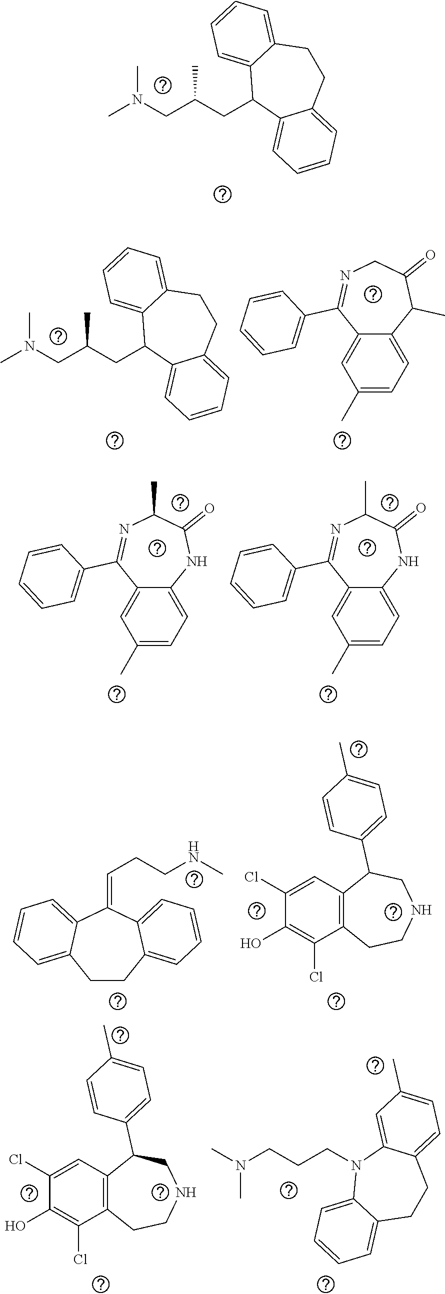

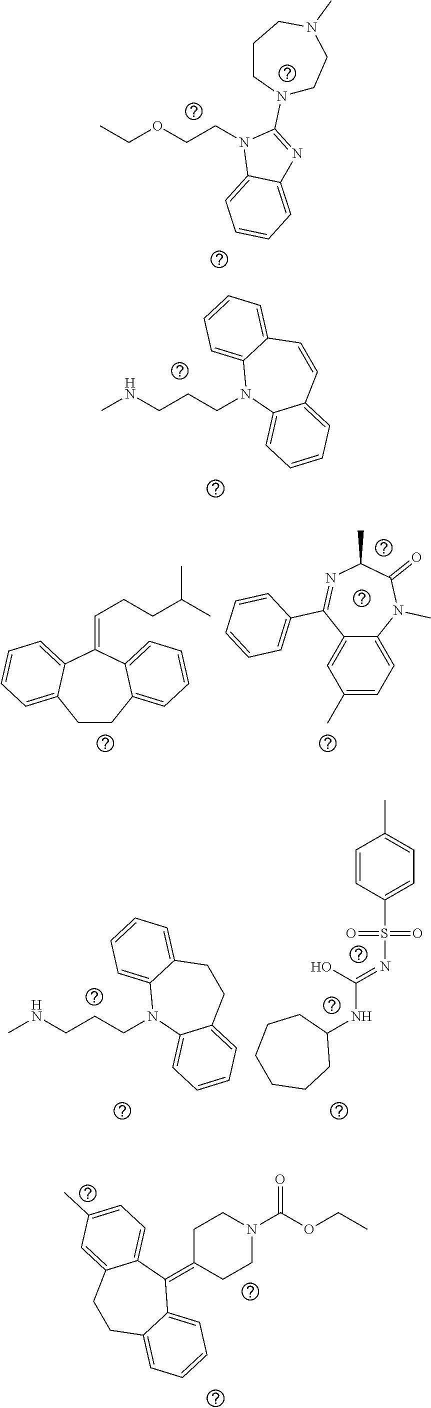

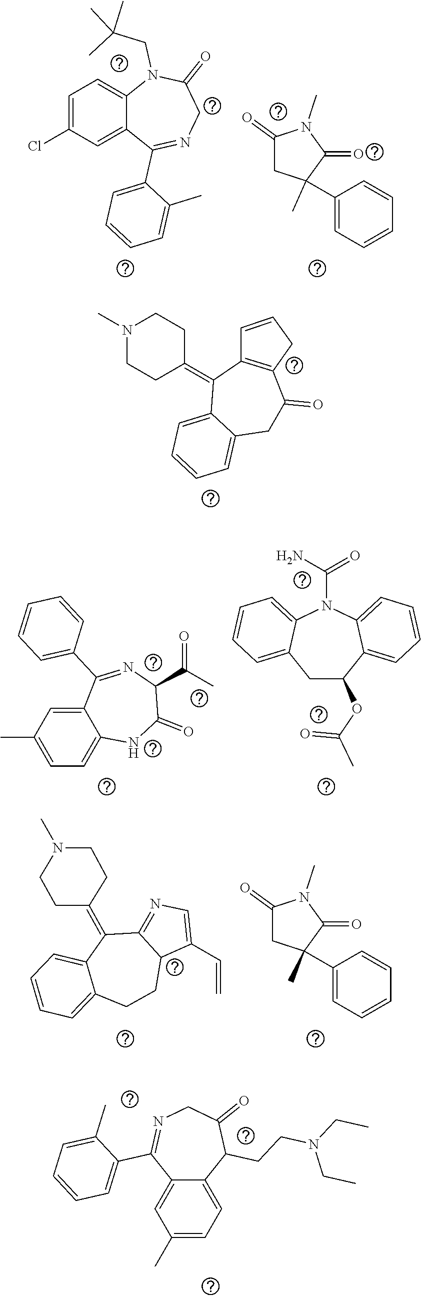

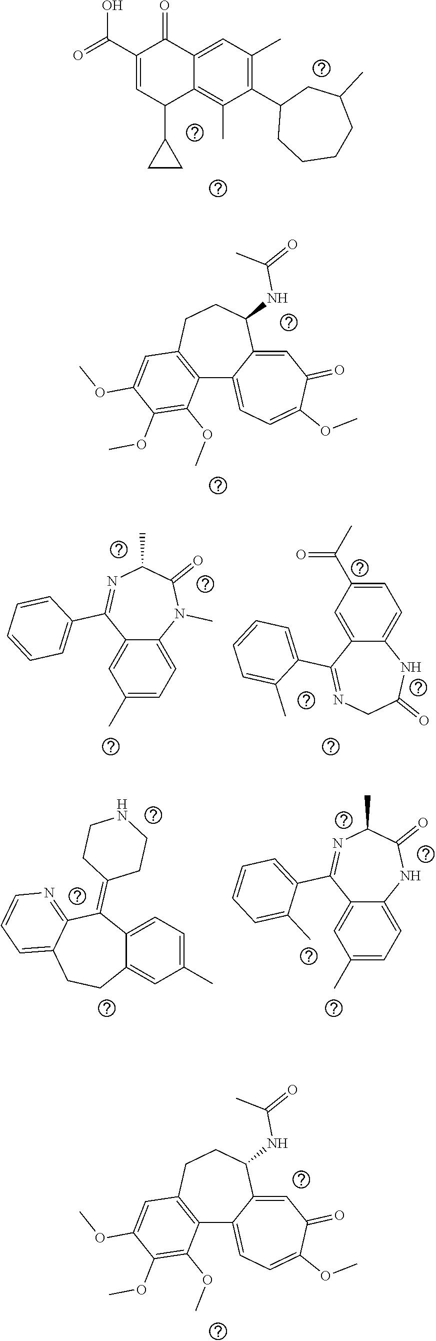

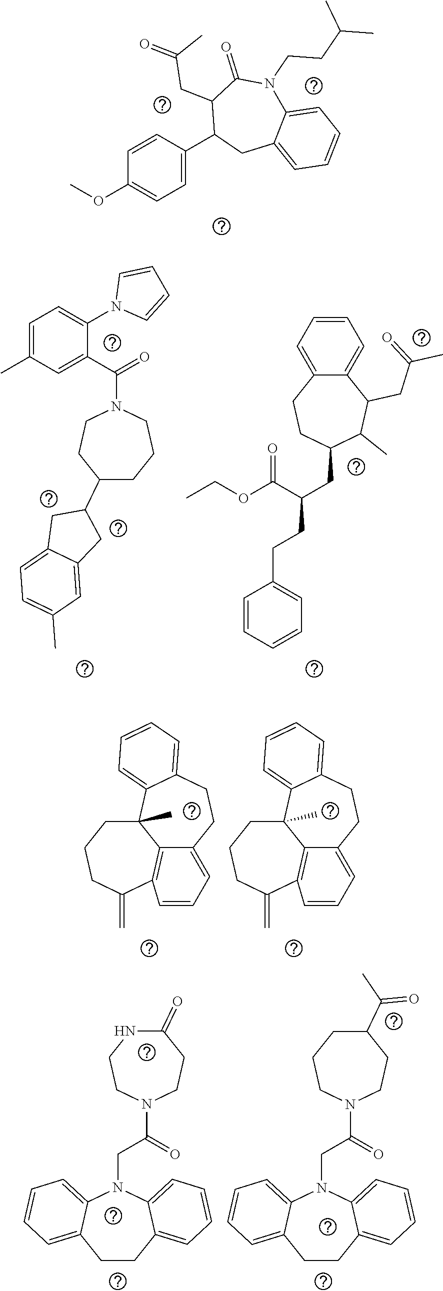

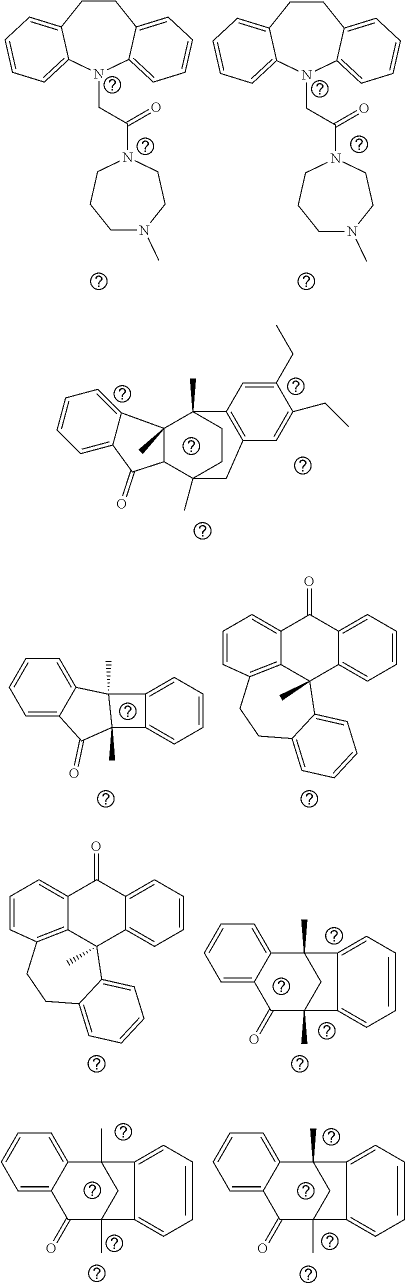

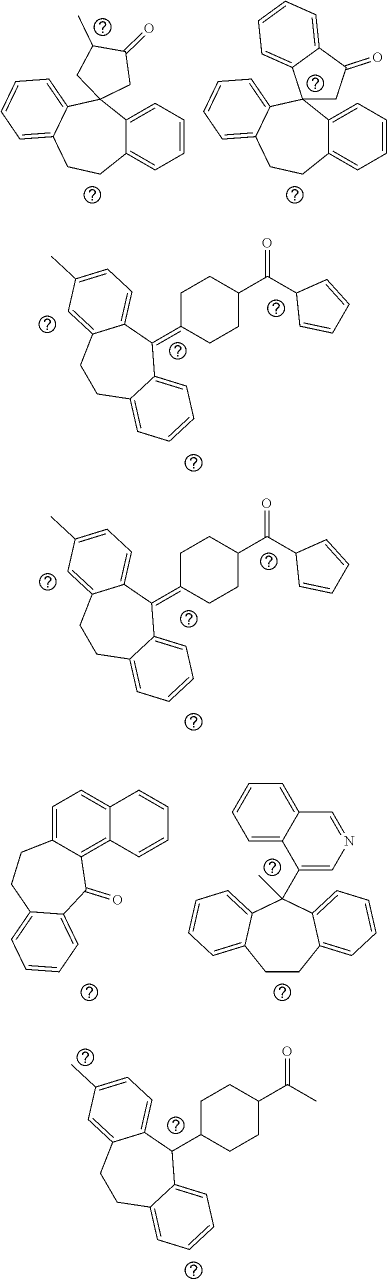

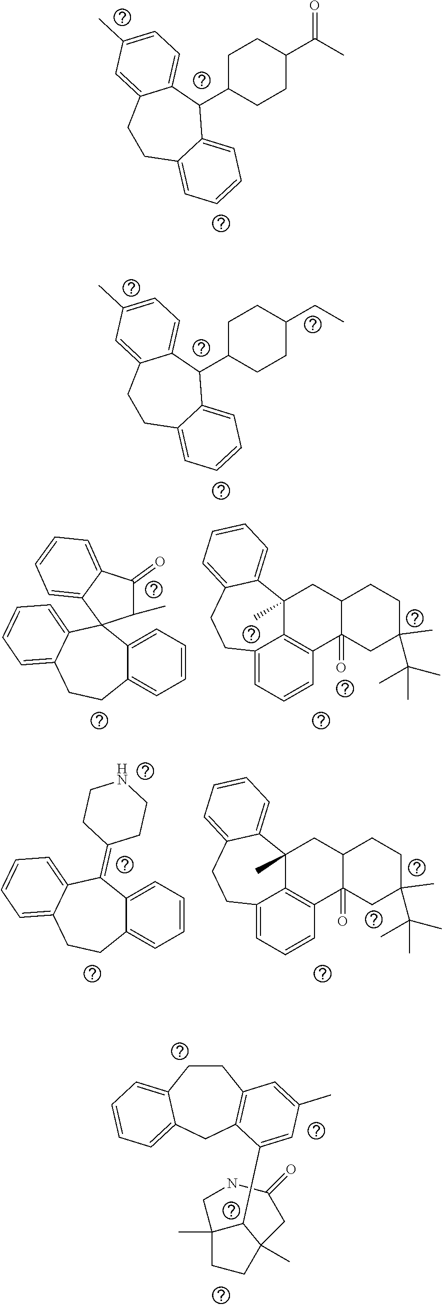

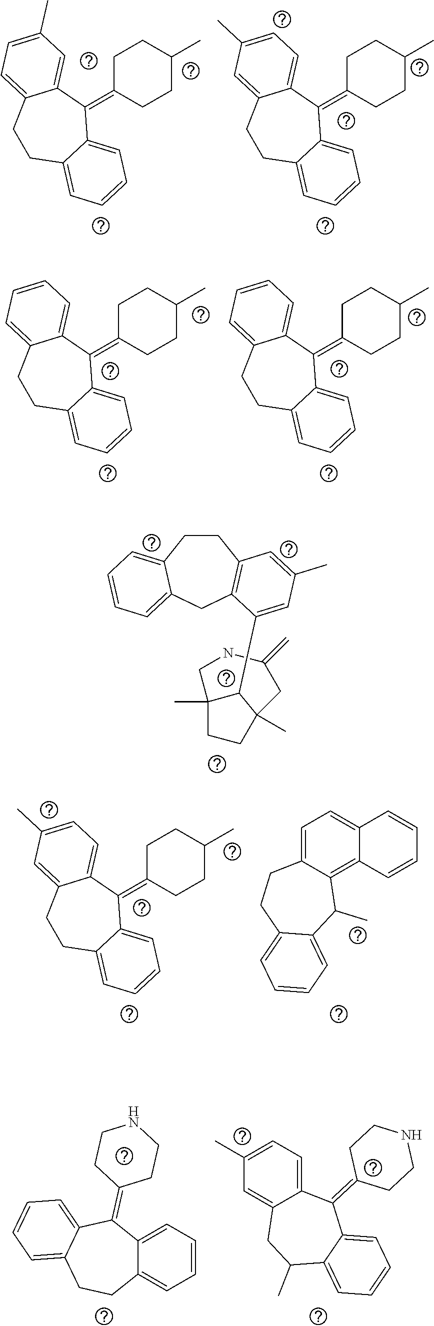

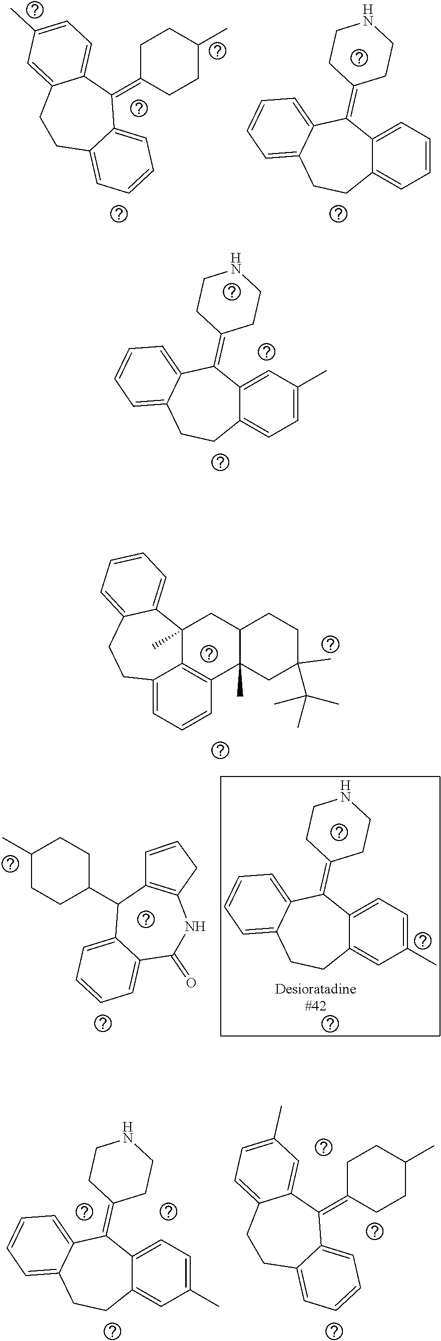

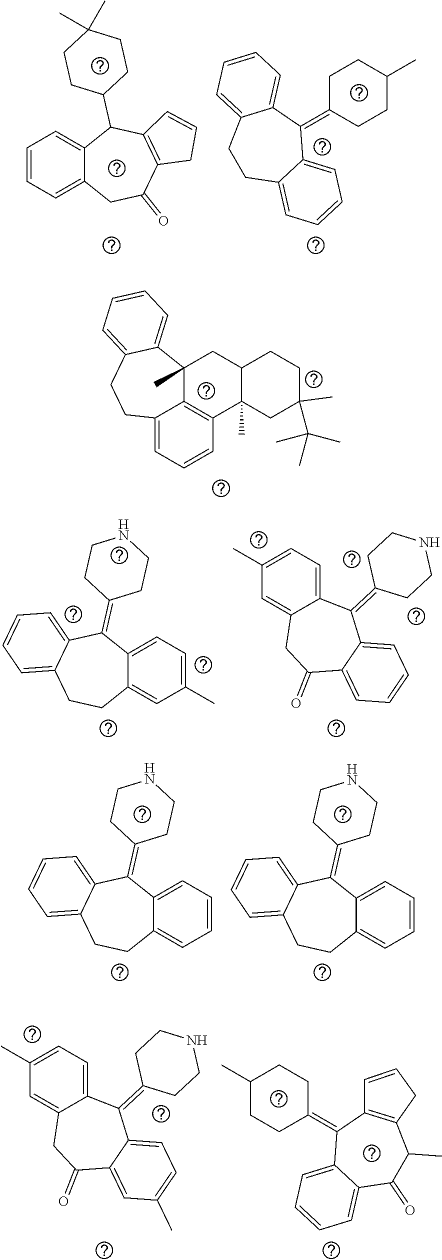

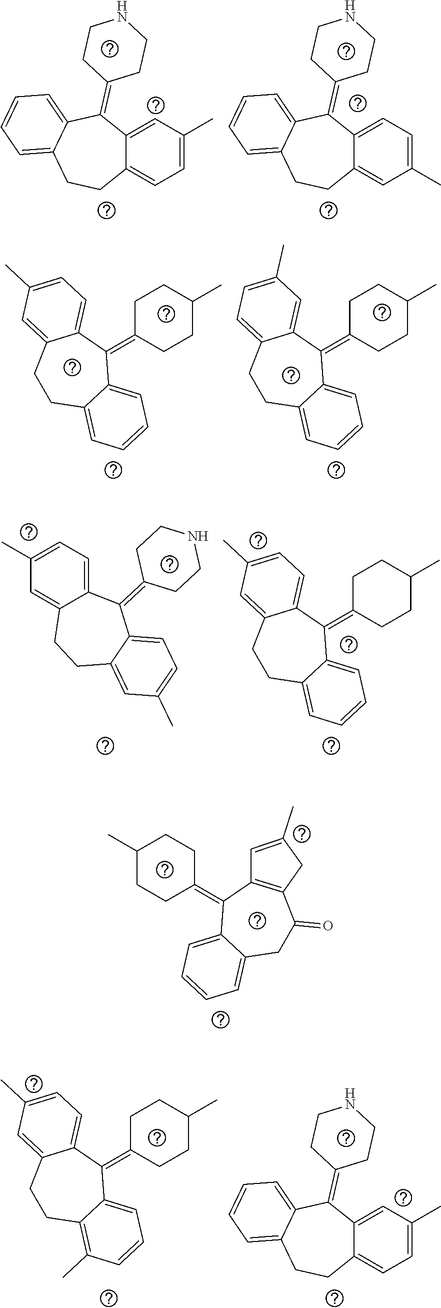

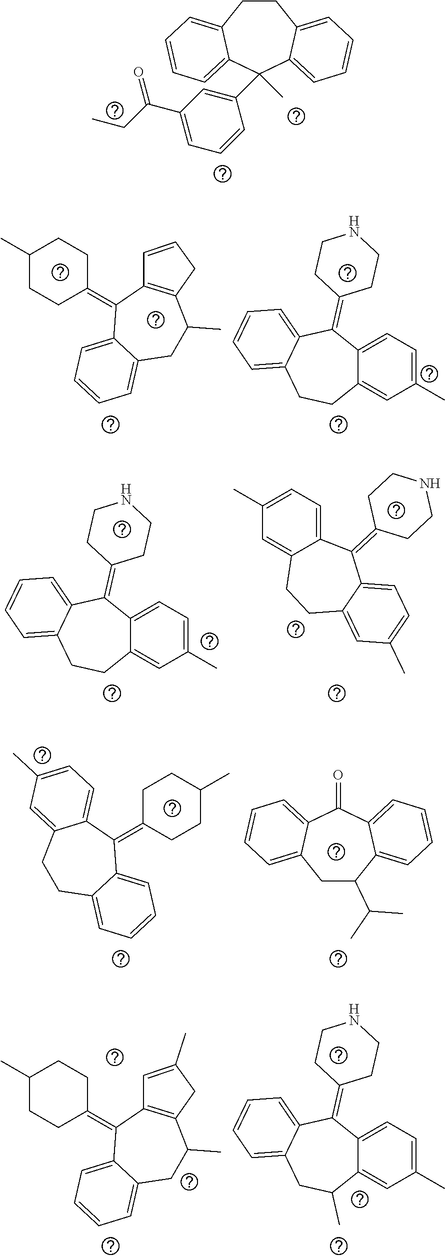

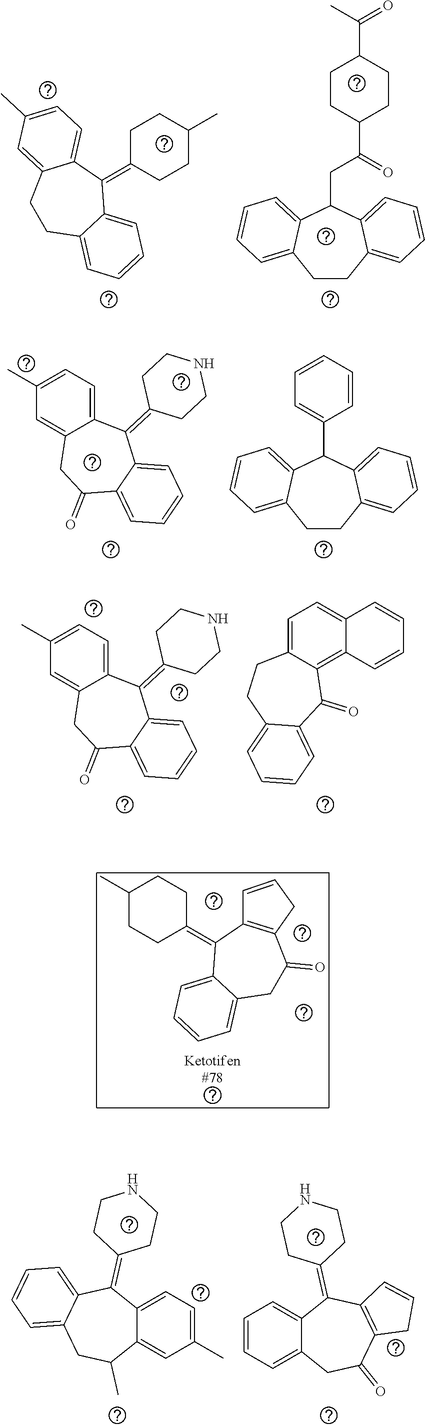

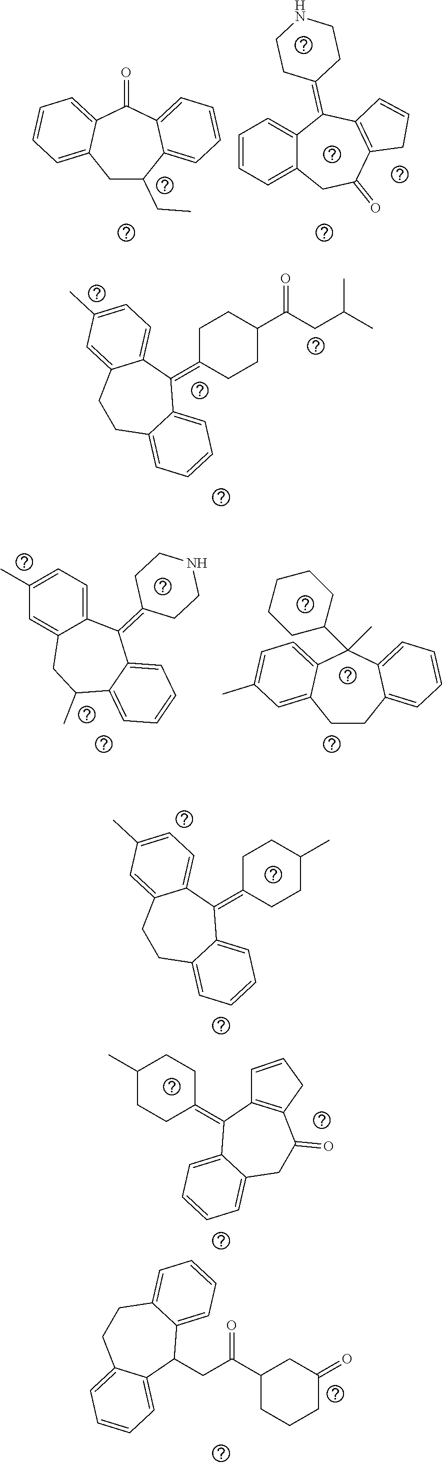

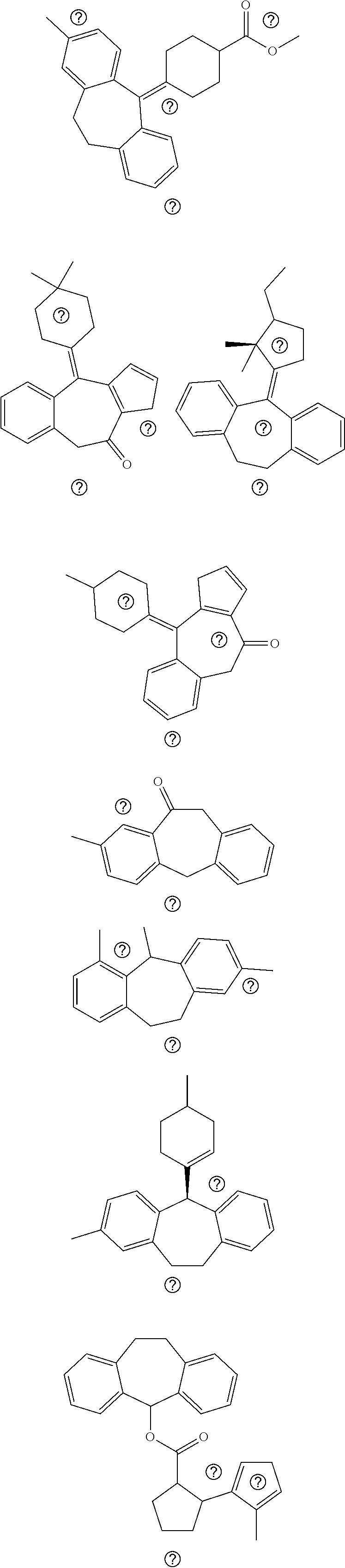

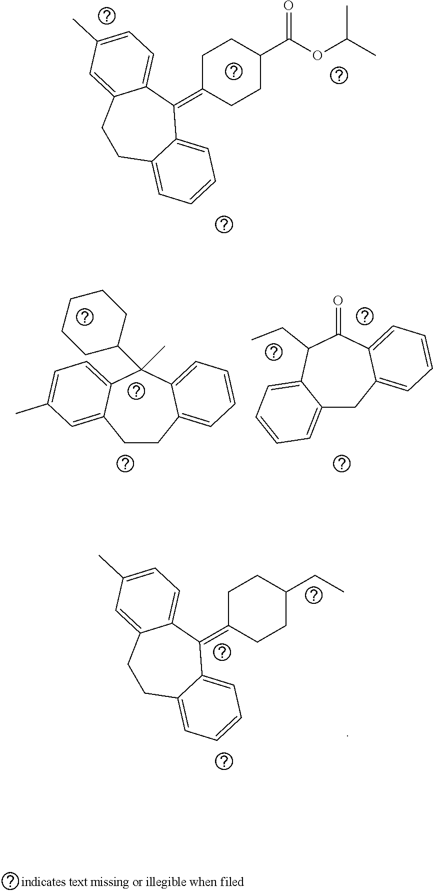

### DESCRIPTION OF DRAWINGS

FIGS. 1A-1H. AKT/IWS1 axis is active in human LPS and associates with poor prognosis. (1A) Western blot of AKT1, AKT2, AKT3, phospho-AKT (Thr 308), phospho-AKT (Ser 473), IWS1 and phospho-IWS1 in LPS tumor tissues and adjacent adipose tissues. (1B) Western blot analysis the expression of phospho-AKT (Thr 308) and phospho-AKT (Ser 473) in LPS tumor tissues and adjacent adipose tissues. (1C) Western blot analysis the expression of IWS1 and phospho-IWS1 in LPS tumor tissues and adjacent adipose tissues. (1D) Heatmap of the correlation coefficients between the indicated components of the AKT/IWS1 phosphorylation pathway. (1E) The relative stoichiometry of IWS1 in the same LPS tumor correlates with phospho-Akt (Thr 308) expression. (1F) The relative stoichiometry of IWS1 in the same LPS tumor correlates with phospho-AKT (Ser 473) expression. (1G) The relative stoichiometry of IWS1 phosphorylation in the same LPS tumor correlates with phospho-Akt (Thr 308) expression. (1H) Immunohistochemical staining of phospho-AKT (Thr 308), phospho-AKT (Ser 473) and IWS1 in WDLPS and DDLPS tissues.

FIGS. 2A-2B. Role of IWS1 on the overall survival analysis of patients with DDLPS. (2A) Kaplan Meier overall survival analysis based on RNA-seq data of patients with DDLPS (n=58) from the TCGA showed a significant reduction in overall survival (OS) and in (2B) disease-specific survival (DSS) in patients with a higher IWS1 expression (higher than the median expression of IWS1 calculated across all samples) compared to those with a lower IWS1 expression (lower than the median, p=0.014, and p=0.005). **P<0.01, ***P<0.001 (log rank p test).

FIGS. 3A-3F. IWS1 phosphorylation at Ser720/Thr721 promote LPS colony forming capacities. (3A) CCK8 assay detected the cell viability of SW872 cells with shcontrol, shIWS1, IWS1 wt, shIWS1/wt rescue or shIWS1/mut rescue transduction. (3B) CCK8 assay detected the cell viability of Lipo863 cells transduced with shcontrol, shIWS1, IWS1 WT, shIWS1/WT-R or shIWS1/MT-R. (3C) Plate colony forming assay detected the colony forming capacities of SW872 and Lipo863 cells transduced with shcontrol, shIWS1, IWS1 WT, shIWS1/WT-R or shIWS1/MT-R. (3D) Plate colony forming analysis of the colony forming capacities in SW872 and Lipo863 cells. (3E) Soft agar colony forming assay detected the colony forming capacities of SW872 and Lipo863 cells transduced with shcontrol, shIWS1, IWS1 WT, shIWS1/WT-R or shIWS1/MT-R. (3F) Soft agar colony forming analysis of the colony forming capacities in SW872 and Lipo863 cells. **P<0.01, ***P<0.001.

FIGS. 4A-4F. IWS1 phosphorylation at Ser720/Thr721 promote LPS cell migration and invasion. (4A) Wound healing assay was conducted to detect the migratory ability of SW872 and Lipo863 cells transduced with shcontrol, shIWS1, IWS1 WT, shIWS1/WT-R or shIWS1/MT-R. (4B) Wound healing analysis of the migratory ability in SW872 cells. (4C) Wound healing analysis of the migratory ability in Lipo863 cells. (4D) Transwell assays were performed to evaluate the migratory and invasive capacities of SW872 and Lipo863 cells transduced with shcontrol, shIWS1, IWS1 WT, shIWS1/ WT-R or shIWS1/ MT-R. (4E) Transwell assays analysis of the migratory and invasive ability in SW872 cells. (4F) Transwell assays analysis of the migratory and invasive ability in Lipo863 cells. **P<0.05, **P<0.01, ***P<0.001.

FIGS. 5A-5I. IWS1 phosphorylation at Ser720/Thr721 promotes tumor growth in vivo, while IWS1 knockdown inhibited tumor growth and metastasis in vivo. (5A) Xenograft LPS model of nude mice inoculated with shcontrol and shlWS1 SW872 cells. (5B) Tumor growth curves of nude mice inoculated with shcontrol and shIWS1 SW872 cells. (5C) Tumor weights of nude mice inoculated with shcontrol and shlWS1 SW872 cells. (5D) Xenograft LPS model of nude mice inoculated with shIWS1/WT-R and shIWS1/MT-R SW872 cells. (5E) Tumor growth curves of nude mice inoculated with shIWS1/ WT-R and shIWS1/MT-R SW872 cells. (5F) Tumor weights of nude mice inoculated with shIWS1/WT-R and shIWS1/MT-R SW872 cells. (5G) LPS metastasis model of nude mice with tail vein injection of shcontrol and shIWS1 SW872 cells. (5H) H&E staining of the lung metastatic nodules in LPS metastasis model of nude mice. (5I) Lung metastatic nodules analysis in LPS metastasis model of nude mice.

FIGS. 6A-6F. IWS1 phosphorylation at Ser720/Thr721 represses MET and promotes cancer stem cell-like properties in LPS cells. (6A) Western blot of the MET-associated markers E-cad, Occludin, N-cad, VIM, Snail and Slug in SW872 cells transduced with shcontrol, shIWS1, IWS1 WT, shIWS1/WT-R or shIWS1/MT-R. (6B) Graphical representation of Western blot showing the expression of MET-associated marker E-cad, Occludin, N-cad, VIM, Snail and Slug in SW872 cells. (6C) Flow cytometry was performed to evaluate the ALDH⁺/CD133⁺ subpopulations in SW872 cells transduced with shcontrol, shIWS1, IWS1 WT, shIWS1/WT-R or shIWS1/MT-R. (6D) Flow cytometry analysis of ALDH⁺/CD133⁺ subpopulations in SW872 cells. (6E) Western blot of CSC marker KLF4, Nanog, OCT4 and Sox2 in SW872 cells. (6F) Western blot analysis the expression of CSC marker KLF4, Nanog, OCT4 and Sox2 in SW872 cells. **P<0.01, ***P<0.001.

FIG. 7. Clinicopathologic variables of the patients with resected retroperitoneal liposarcomas (n=33) used in the study in Example 1.

FIGS. 8A-8E. AKT/IWS1 axis is active in human liposarcoma. (8A) Western blot of AKT1, AKT2, AKT3, phospho-AKT (Thr 308), phospho-AKT (Ser 473), pan-AKT, IWS1, and phospho-IWS1 in LPS tumor tissues and adjacent adipose tissues. (8B) Western blot analysis of the expression of phospho-AKT (Thr 308) and phospho-AKT (Ser 473) in WDLPS and DDLPS tissues. (8C) Western blot analysis of the expression of IWS1 and phospho-IWS1 in WDLPS and DDLPS tissues. (8D) Western blot analysis of the expression of AKT1, AKT2, and AKT3 in LPS tumor tissues and adjacent adipose tissues. (8E) Western blot analysis of the expression of AKT1, AKT2 and AKT3 in WDLPS and DDLPS tissues. *P<0.05, **P<0.01, ***P<0.001.

FIGS. 9A-9F. The mRNA and protein expression of IWS1 in the SW872 and Lipo863 cells after transduction. (9A) qRT-PCR was conducted to detect the mRNA expression of IWS1 in SW872 cells transduced with shcontrol, shIWS1, IWS1 WT, shIWS1/WT-R or shIWS1/MT-R. (9B) qRT-PCR was conducted to detect the mRNA expression of IWS1 in Lipo863 cells transduced with shcontrol, shIWS1, IWS1 WT, shIWS1/WT-R or shIWS1/MT-R. (9C) Western blot of IWS1 in SW872 cells transduced with shcontrol, shIWS1, IWS1 WT, shIWS1/WT-R or shIWS1/MT-R. (9D) Western blot of IWS1 in Lipo863 cells transduced with shcontrol, shIWS1, IWS1 WT, shIWS1/WT-R or shIWS1/MT-R. *P<0.05, **P<0.01, ***P<0.001. (9E) Western blot analysis of the expression of IWS1 in SW872 cells. (9F) Western blot analysis of the expression of IWS1 in Lipo863 cells. *P<0.05, **P<0.01, ***P<0.001.

FIGS. 10A-10B. IWS1 knock-down induces cell cycle arrest. (10A) Flow cytometry analysis of Lipo863, Lipo815, and Lipo224 cells transduced with shcontrol and shIWS1. Graphical representation of flow cytometry analysis showing the accumulation of cells in the S phase of the cell cycle in all three cell lines that were transduced with shIWS1. *P<0.05, **P<0.01, ***P<0.001. (10B) qRT-PCR was conducted to detect the mRNA expression of S phase checkpoint genes, CDK2 and CCNA2 (Cyclin A2) on Lipo863, Lipo815, and Lipo224 cells transduced with shcontrol, and shIWS1. *P<0.05, **P<0.01, ***P<0.001.

FIGS. 11A-11C. IWS1 promotes liposarcoma cell migration and invasion. (11A) Transwell assays were performed to evaluate the migratory and invasive capacities of Lipo224 and Lipo815 cells transduced with shcontrol, shIWS1, or IWS1 WT. (11B) Transwell assays analysis of the migratory and invasive ability in Lipo224 cells. (11C) Transwell assays analysis of the migratory and invasive ability in Lipo815 cells. *P<0.05, **P<0.01, ***P<0.001.

FIGS. 12A-12F. IWS1 phosphorylation at Ser720/Thr721 promotes tumor growth in vivo. (12A) Immunohistochemical staining of Ki 67 in tumor xenografts. (12B) Graphical representation of Immunohistochemical staining of Ki 67 in tumor xenografts. **P<0.01, ***P<0.001. (12C) Western blot of IWS1, MET-associated markers E-cad, Occludin, N-cad, VIM, Snail and Slug as well CSC marker KLF4, Nanog, OCT4 and Sox2 and expression of CSC markers KLF4, Nanog, OCT4 and Sox2 in tumor xenografts. (12D) Graphical representation of Western blot analysis showing expression of the MET-associated markers E-cad, Occludin, N-cad, VIM, Snail and Slug in tumor xenografts. (12E) Graphical representation of Western blot analysis showing the expression of IWS1 and the expression of CSC markers Sox2, Nanog, KLF4, and OCT4 in tumor xenografts. (12F) Immunohistochemical staining of VIM and pan Cytokeratin in tumor xenografts. **P<0.01, ***P<0.001.

FIG. 13. Shows the structure of FDA-approved compounds identified as hits from in silico screen for potential IWS1 inhibitors.

FIG. 14. Shows the structure of the top 100 commercially available compounds identified as hits from in silico screen for potential IWS1 inhibitors.

FIG. 15A-15C. Evaluation of the cytotoxicity of IWS1 inhibitors identified from in silico screening using dedifferentiated liposarcoma (DDLPS) cell line Lipo863 as an in-vitro model. IC50 values were determined using MTS assay (the concentration at which the drug kills 50% of cancer cells). The IC50 values are 14, 33, and 63 μM for Desloratadine (FIG. 15A), Cyproheptadine (FIG. 15B), and Ketotifen (FIG. 15C), respectively. These curves were used determine the concentration range of each drug for further experiments in which the drugs did not cause any cell death so as to allow for the study the inhibitory effect on IWS1 while minimizing off-target effects.

FIG. 16A-16B. Evaluation of the effect of IWS1 inhibitors identified from in silico screening on the migration of cancer cells. One of the effects on cell phenotype of IWS1 knockdown is a decrease in cell migration. Treating cells with a potential IWS1 inhibitor was expect to mimic/reproduce this phenotype. The migration assay was performed using two cell groups: Empty Vector (EV, Negative control) and shIWS1 (IWS1 knockdown). We expected to see a reduction in migration in shIWS1 vs EV (knockdown effect) and in treatment vs non-treatment in the EV group (pharmacological IWS1 inhibition). We expected a non-significant reduction in migration in treatment vs non-treatment in the shIWS1 group suggesting IWS1 presence as a key factor causing this phenotype. All 3 expectations were met. All 3 drugs show a reduction in cell migration for the Empty Vector (EV): Desloratadine −29%, Cyproheptadine −24%, and Ketotifen −18%. There was no significant reduction in migration for shIWS1 (comparing the drugs to the negative control). A significant reduction (~61%) in cell migration in the shIWS1 samples was observed compared to the EV samples.

FIG. 17. Evaluation of the dose-dependent effect of IWS1 inhibitors identified from in silico screening on the migration of cancer cells. Cell migration in vitro represents to some extent the ability of cells to metastasize in vivo. A dose-dependent effect on migration in vitro shows the potential of the drug to work in vivo and at lower concentrations. The pharmacological inhibition was expected to show a progressive effect if the plateau has not been reached.

FIG. 18. Evaluation of the effect of IWS1 inhibitors identified from in silico screening on the migration of cancer cells. Invasiveness is important parameter used to characterize the aggressive behavior of cancer cells. To test the effect of Ketotifen and Desloratadine on the invasiveness of cells, invasive DDLPS cell line Lipo246 was used. After incubating cells for 48 hours at doses of 20 μM and 4 μM of Ketotifen and Desloratadine, respectively, a significant decrease in invasion ability of Lipo246 cells was observed for both Ketotifen and Desloratadine (30% and 25%, respectively).

FIG. 19. To evaluate the impact of IWS1 inhibition on spheroid formation, a matrigel extracellular matrix scaffold was employed. 14-day treatment with Ketotifen or Desloratadine significantly reduced spheroid formation (both the number and size of spheroids), confirming their impact on tumor growth and cell proliferation. FIG. 19 shows representative microscopic images of spheroids of Lipo863 cells after 14-day treatment with Ketotifen and Desloratadine, ***p<0.001, ****p<0.0001.

### DETAILED DESCRIPTION

Before the present methods and systems are disclosed and described, it is to be understood that the methods and systems are not limited to specific synthetic methods, specific components, or to particular compositions. It is also to be understood that the terminology used herein is for the purpose of describing particular embodiments only and is not intended to be limiting.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

As used in this specification and the following claims, the terms “comprise” (as well as forms, derivatives, or variations thereof, such as “comprising” and “comprises”) and “include” (as well as forms, derivatives, or variations thereof, such as “including” and “includes”) are inclusive (i.e., open-ended) and do not exclude additional elements or steps. For example, the terms “comprise” and/or “comprising,” when used in this specification, specify the presence of stated features, integers, steps, operations, elements, and/or components, but do not preclude the presence or addition of one or more other features, integers, steps, operations, elements, components, and/or groups thereof. Other than where noted, all numbers expressing quantities of ingredients, reaction conditions, geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

Accordingly, these terms are intended to not only cover the recited element(s) or step(s), but may also include other elements or steps not expressly recited. Furthermore, as used herein, the use of the terms “a”, “an”, and “the” when used in conjunction with an element may mean “one,” but it is also consistent with the meaning of “one or more,” “at least one,” and “one or more than one.” Therefore, an element preceded by “a” or “an” does not, without more constraints, preclude the existence of additional identical elements.

Ranges can be expressed herein as from “about” one particular value, and/or to “about” another particular value. By “about” is meant within 5% of the value, e.g., within 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. A range may be construed to include the start and the end of the range. For example, a range of 10% to 20% (i.e., range of 10%-20%) can includes 10% and also includes 20%, and includes percentages in between 10% and 20%, unless explicitly stated otherwise herein.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

It is understood that when combinations, subsets, groups, etc. of elements are disclosed (e.g., combinations of components in a composition, or combinations of steps in a method), that while specific reference of each of the various individual and collective combinations and permutations of these elements may not be explicitly disclosed, each is specifically contemplated and described herein.

“Administration” to a subject includes any route of introducing or delivering to a subject an agent. Administration can be carried out by any suitable route, including oral, topical, transcutaneous, transdermal, intra-joint, intra-arteriole, intradermal, intraventricular, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration but are undetectable or detectable at negligible amounts in distal parts of the subject's body. Administration includes self-administration and the administration by another.

As used herein, the term “controlled-release” or “controlled-release drug delivery” or “extended release” refers to release or administration of a drug from a given dosage form in a controlled fashion in order to achieve the desired pharmacokinetic profile in vivo. An aspect of “controlled” drug delivery is the ability to manipulate the formulation and/or dosage form in order to establish the desired kinetics of drug release.

As used here, the terms “beneficial agent” and “active agent” are used interchangeably herein to refer to a chemical compound or composition that has a beneficial biological effect. Beneficial biological effects include both therapeutic effects, i.e., treatment of a disorder or other undesirable physiological condition, and prophylactic effects, i.e., prevention of a disorder or other undesirable physiological condition. The terms also encompass pharmaceutically acceptable, pharmacologically active derivatives of beneficial agents specifically mentioned herein, including, but not limited to, salts, esters, amides, prodrugs, active metabolites, isomers, fragments, analogs, and the like. When the terms “beneficial agent” or “active agent” are used, then, or when a particular agent is specifically identified, it is to be understood that the term includes the agent per se as well as pharmaceutically acceptable, pharmacologically active salts, esters, amides, prodrugs, conjugates, active metabolites, isomers, fragments, analogs, etc.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the term “therapeutic agent” is used, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

“Inactivate”, “inactivating” and “inactivation” means to decrease or eliminate an activity, response, condition, disease, or other biological parameter due to a chemical (covalent bond formation) between the ligand and a its biological target.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

As used herein, the terms “treating” or “treatment” of a subject includes the administration of a drug to a subject with the purpose of preventing, curing, healing, alleviating, relieving, altering, remedying, ameliorating, improving, stabilizing or affecting a disease or disorder, or a symptom of a disease or disorder. The terms “treating” and “treatment” can also refer to reduction in severity and/or frequency of symptoms, elimination of symptoms and/or underlying cause, prevention of the occurrence of symptoms and/or their underlying cause, and improvement or remediation of damage.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed. For example, the terms “prevent” or “suppress” can refer to a treatment that forestalls or slows the onset of a disease or condition or reduced the severity of the disease or condition. Thus, if a treatment can treat a disease in a subject having symptoms of the disease, it can also prevent or suppress that disease in a subject who has yet to suffer some or all of the symptoms. As used herein, the term “preventing” a disorder or unwanted physiological event in a subject refers specifically to the prevention of the occurrence of symptoms and/or their underlying cause, wherein the subject may or may not exhibit heightened susceptibility to the disorder or event.

By the term “effective amount” of a therapeutic agent is meant a nontoxic but sufficient amount of a beneficial agent to provide the desired effect. The amount of beneficial agent that is “effective” will vary from subject to subject, depending on the age and general condition of the subject, the particular beneficial agent or agents, and the like. Thus, it is not always possible to specify an exact “effective amount”. However, an appropriate “effective’ amount in any subject case may be determined by one of ordinary skill in the art using routine experimentation. Also, as used herein, and unless specifically stated otherwise, an “effective amount” of a beneficial can also refer to an amount covering both therapeutically effective amounts and prophylactically effective amounts.

An “effective amount” of a drug necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

As used herein, a “therapeutically effective amount” of a therapeutic agent refers to an amount that is effective to achieve a desired therapeutic result, and a “prophylactically effective amount” of a therapeutic agent refers to an amount that is effective to prevent an unwanted physiological condition. Therapeutically effective and prophylactically effective amounts of a given therapeutic agent will typically vary with respect to factors such as the type and severity of the disorder or disease being treated and the age, gender, and weight of the subject. The term “therapeutically effective amount” can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the drug and/or drug formulation to be administered (e.g., the potency of the therapeutic agent (drug), the concentration of drug in the formulation, and the like), and a variety of other factors that are appreciated by those of ordinary skill in the art.

As used herein, the term “pharmaceutically acceptable” component can refer to a component that is not biologically or otherwise undesirable, i.e., the component may be incorporated into a pharmaceutical formulation of the invention and administered to a subject as described herein without causing any significant undesirable biological effects or interacting in a deleterious manner with any of the other components of the formulation in which it is contained. When the term “pharmaceutically acceptable” is used to refer to an excipient, it is generally implied that the component has met the required standards of toxicological and manufacturing testing or that it is included on the Inactive Ingredient Guide prepared by the U.S. Food and Drug Administration.

Also, as used herein, the term “pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

A “control” is an alternative subject or sample used in an experiment for comparison purposes. A control can be “positive” or “negative.”

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, goats, sheep, pigs, dogs, cats, etc.), and birds (e.g., chickens, turkeys, songbirds, etc.). “Subject” can also include a mammal, such as a primate or a human. Thus, the subject can be a human or veterinary patient. The term “patient” refers to a subject under the treatment of a clinician, e.g., physician. Administration of the therapeutic agents can be carried out at dosages and for periods of time effective for treatment of a subject. In some embodiments, the subject is a human.

The term “n-membered” where n is an integer typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

As used herein, the phrase “optionally substituted” means unsubstituted or substituted. As used herein, the term “substituted” means that a hydrogen atom is removed and replaced by a substituent. It is to be understood that substitution at a given atom is limited by valency.

Throughout the definitions, the term “Cₙ₋ₘ” indicates a range which includes the endpoints, wherein n and m are integers and indicate the number of carbons. Examples include C₁₋₄, C₁₋₆, and the like.

As used herein, the term “Cₙ₋ₘ alkyl”, employed alone or in combination with other terms, refers to a saturated hydrocarbon group that may be straight-chain or branched, having n to m carbons. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms.

As used herein, “Cₙ₋ₘ alkenyl” refers to an alkyl group having one or more double carbon-carbon bonds and having n to m carbons. Example alkenyl groups include, but are not limited to, ethenyl, n-propenyl, isopropenyl, n-butenyl, sec-butenyl, and the like. In some embodiments, the alkenyl moiety contains 2 to 6, 2 to 4, or 2 to 3 carbon atoms.

As used herein, “Cₙ₋ₘ alkynyl” refers to an alkyl group having one or more triple carbon-carbon bonds and having n to m carbons. Example alkynyl groups include, but are not limited to, ethynyl, propyn-1-yl, propyn-2-yl, and the like. In some embodiments, the alkynyl moiety contains 2 to 6, 2 to 4, or 2 to 3 carbon atoms.

As used herein, the term “Cₙ₋ₘ alkylene”, employed alone or in combination with other terms, refers to a divalent alkyl linking group having n to m carbons. Examples of alkylene groups include, but are not limited to, ethan-1,2-diyl, propan-1,3-diyl, propan-1,2-diyl, butan-1,4-diyl, butan-1,3-diyl, butan-1,2-diyl, 2-methyl-propan-1,3-diyl, and the like. In some embodiments, the alkylene moiety contains 2 to 6, 2 to 4, 2 to 3, 1 to 6, 1 to 4, or 1 to 2 carbon atoms.

As used herein, the term “Cₙ₋ₘ alkoxy”, employed alone or in combination with other terms, refers to a group of formula —O-alkyl, wherein the alkyl group has n to m carbons. Example alkoxy groups include methoxy, ethoxy, propoxy (e.g., n-propoxy and isopropoxy), tert-butoxy, and the like. In some embodiments, the alkyl group has 1 to 6, 1 to 4, or 1 to 3 carbon atoms.

As used herein, the term “Cₙ₋ₘ alkylamino” refers to a group of formula —NH(alkyl), wherein the alkyl group has n to m carbon atoms. In some embodiments, the alkyl group has 1 to 6, 1 to 4, or 1 to 3 carbon atoms.

As used herein, the term “Cₙ₋ₘ alkoxycarbonyl” refers to a group of formula —C(O)O-alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylcarbonyl” refers to a group of formula —C(O)— alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylcarbonylamino” refers to a group of formula —NHC(O)-alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylsulfonylamino” refers to a group of formula —NHS(O)₂-alkyl, wherein the alkyl group has n to m carbon atoms. In some embodiments, the alkyl group has 1 to 6, 1 to 4, or 1 to 3 carbon atoms.

As used herein, the term “aminosulfonyl” refers to a group of formula —S(O)₂NH₂.

As used herein, the term “Cₙ₋ₘ alkylaminosulfonyl” refers to a group of formula —S(O)₂NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ alkyl)aminosulfonyl” refers to a group of formula —S(O)₂N(alkyl)₂, wherein each alkyl group independently has n to m carbon atoms.

In some embodiments, each alkyl group has, independently, 1 to 6, 1 to 4, or 1 to 3 carbon atoms.

As used herein, the term “aminosulfonylamino” refers to a group of formula —NHS(O)₂NH₂.

As used herein, the term “Cₙ₋ₘ alkylaminosulfonylamino” refers to a group of formula —NHS(O)₂NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ alkyl)aminosulfonylamino” refers to a group of formula —NHS(O)₂N(alkyl)₂, wherein each alkyl group independently has n to m carbon atoms. In some embodiments, each alkyl group has, independently, 1 to 6, 1 to 4, or 1 to 3 carbon atoms.

As used herein, the term “aminocarbonylamino”, employed alone or in combination with other terms, refers to a group of formula —NHC(O)NH₂.

As used herein, the term “Cₙ₋ₘ alkylaminocarbonylamino” refers to a group of formula —NHC(O)NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ alkyl)aminocarbonylamino” refers to a group of formula —NHC(O)N(alkyl)₂, wherein each alkyl group independently has n to m carbon atoms. In some embodiments, each alkyl group has, independently, 1 to 6, 1 to 4, or 1 to 3 carbon atoms.

As used herein, the term “Cₙ₋ₘ alkylcarbamyl” refers to a group of formula —C(O)—NH(alkyl), wherein the alkyl group has n to m carbon atoms. In some embodiments, the alkyl group has 1 to 6, 1 to 4, or 1 to 3 carbon atoms.

As used herein, the term “thio” refers to a group of formula —SH.

As used herein, the term “Cₙ₋ₘ alkylsulfinyl” refers to a group of formula —S(O)— alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylsulfonyl” refers to a group of formula —S(O)₂-alkyl, wherein the alkyl group has n to m carbon atoms. In some embodiments, the alkyl group has 1 to 6, 1 to 4, or 1 to 3 carbon atoms.

As used herein, the term “amino” refers to a group of formula —NH₂.

As used herein, the term “aryl,” employed alone or in combination with other terms, refers to an aromatic hydrocarbon group, which may be monocyclic or polycyclic (e.g., having 2, 3 or 4 fused rings). The term “Cₙ₋ₘ aryl” refers to an aryl group having from n to m ring carbon atoms. Aryl groups include, e.g., phenyl, naphthyl, anthracenyl, phenanthrenyl, indanyl, indenyl, and the like. In some embodiments, aryl groups have from 6 to about 20 carbon atoms, from 6 to about 15 carbon atoms, or from 6 to about 10 carbon atoms. In some embodiments, the aryl group is a substituted or unsubstituted phenyl.

As used herein, the term “carbamyl” to a group of formula —C(O)NH₂.

As used herein, the term “carbonyl”, employed alone or in combination with other terms, refers to a —C(═O)— group, which may also be written as C(O).

As used herein, the term “di(Cₙ₋ₘ-alkyl)amino” refers to a group of formula —N(alkyl)₂, wherein the two alkyl groups each has, independently, n to m carbon atoms. In some embodiments, each alkyl group independently has 1 to 6, 1 to 4, or 1 to 3 carbon atoms.

As used herein, the term “di(Cₙ₋ₘ-alkyl)carbamyl” refers to a group of formula —C(O)N(alkyl)₂, wherein the two alkyl groups each has, independently, n to m carbon atoms.

In some embodiments, each alkyl group independently has 1 to 6, 1 to 4, or 1 to 3 carbon atoms.

As used herein, the term “halo” refers to F, Cl, Br, or I. In some embodiments, a halo is F, Cl, or Br. In some embodiments, a halo is F or Cl.

As used herein, “Cₙ₋ₘ haloalkoxy” refers to a group of formula —O-haloalkyl having n to m carbon atoms. An example haloalkoxy group is OCF₃. In some embodiments, the haloalkoxy group is fluorinated only. In some embodiments, the alkyl group has 1 to 6, 1 to 4, or 1 to 3 carbon atoms.

As used herein, the term “Cₙ₋ₘ haloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group has 1 to 6, 1 to 4, or 1 to 3 carbon atoms.

As used herein, “cycloalkyl” refers to non-aromatic cyclic hydrocarbons including cyclized alkyl and/or alkenyl groups. Cycloalkyl groups can include mono- or polycyclic (e.g., having 2, 3 or 4 fused rings) groups and spirocycles. Cycloalkyl groups can have 3, 4, 5, 6, 7, 8, 9, or 10 ring-forming carbons (C₃₋₁₀). Ring-forming carbon atoms of a cycloalkyl group can be optionally substituted by oxo or sulfido (e.g., C(O) or C(S)). Cycloalkyl groups also include cycloalkylidenes. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, and the like. In some embodiments, cycloalkyl is cyclopropyl, cyclobutyl, cyclopentyl, cyclohexyl, cyclopentyl, or adamantyl. In some embodiments, the cycloalkyl has 6-10 ring-forming carbon atoms. In some embodiments, cycloalkyl is adamantyl. Also included in the definition of cycloalkyl are moieties that have one or more aromatic rings fused (i.e., having a bond in common with) to the cycloalkyl ring, for example, benzo or thienyl derivatives of cyclopentane, cyclohexane, and the like. A cycloalkyl group containing a fused aromatic ring can be attached through any ring-forming atom including a ring-forming atom of the fused aromatic ring.

As used herein, “heteroaryl” refers to a monocyclic or polycyclic aromatic heterocycle having at least one heteroatom ring member selected from sulfur, oxygen, and nitrogen. In some embodiments, the heteroaryl ring has 1, 2, 3, or 4 heteroatom ring members independently selected from nitrogen, sulfur and oxygen. In some embodiments, any ring-forming N in a heteroaryl moiety can be an N-oxide. In some embodiments, the heteroaryl has 5-10 ring atoms and 1, 2, 3 or 4 heteroatom ring members independently selected from nitrogen, sulfur and oxygen. In some embodiments, the heteroaryl has 5-6 ring atoms and 1 or 2 heteroatom ring members independently selected from nitrogen, sulfur and oxygen. In some embodiments, the heteroaryl is a five-membered or six-membereted heteroaryl ring. A five-membered heteroaryl ring is a heteroaryl with a ring having five ring atoms wherein one or more (e.g., 1, 2, or 3) ring atoms are independently selected from N, O, and S. Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl. A six-membered heteroaryl ring is a heteroaryl with a ring having six ring atoms wherein one or more (e.g., 1, 2, or 3) ring atoms are independently selected from N, O, and S. Exemplary six-membered ring heteroaryls are pyridyl, pyrazinyl, pyrimidinyl, triazinyl and pyridazinyl.

As used herein, “heterocycloalkyl” refers to non-aromatic monocyclic or polycyclic heterocycles having one or more ring-forming heteroatoms selected from O, N, or S. Included in heterocycloalkyl are monocyclic 4-, 5-, 6-, and 7-membered heterocycloalkyl groups. Heterocycloalkyl groups can also include spirocycles. Example heterocycloalkyl groups include pyrrolidin-2-one, 1,3-isoxazolidin-2-one, pyranyl, tetrahydropuran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, benzazapene, and the like. Ring-forming carbon atoms and heteroatoms of a heterocycloalkyl group can be optionally substituted by oxo or sulfido (e.g., C(O), S(O), C(S), or S(O)₂, etc.). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the cycloalkyl ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring. In some embodiments, the heterocycloalkyl has 4-10, 4-7 or 4-6 ring atoms with 1 or 2 heteroatoms independently selected from nitrogen, oxygen, or sulfur and having one or more oxidized ring members.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas a pyridin-3-yl ring is attached at the 3-position.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone—enol pairs, amide—imidic acid pairs, lactam—lactim pairs, enamine—imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

In some embodiments, the compounds described herein can contain one or more asymmetric centers and thus occur as racemates and racemic mixtures, enantiomerically enriched mixtures, single enantiomers, individual diastereomers and diastereomeric mixtures (e.g., including (R)- and (S)-enantiomers, diastereomers, (D)-isomers, (L)-isomers, (+) (dextrorotatory) forms, (−) (levorotatory) forms, the racemic mixtures thereof, and other mixtures thereof). Additional asymmetric carbon atoms can be present in a substituent, such as an alkyl group. All such isomeric forms, as well as mixtures thereof, of these compounds are expressly included in the present description. The compounds described herein can also or further contain linkages wherein bond rotation is restricted about that particular linkage, e.g. restriction resulting from the presence of a ring or double bond (e.g., carbon-carbon bonds, carbon-nitrogen bonds such as amide bonds). Accordingly, all cis/trans and E/Z isomers and rotational isomers are expressly included in the present description. Unless otherwise mentioned or indicated, the chemical designation of a compound encompasses the mixture of all possible stereochemically isomeric forms of that compound.

Optical isomers can be obtained in pure form by standard procedures known to those skilled in the art, and include, but are not limited to, diastereomeric salt formation, kinetic resolution, and asymmetric synthesis. See, for example, Jacques, et al., Enantiomers, Racemates and Resolutions (Wiley Interscience, New York, 1981); Wilen, S. H., et al., Tetrahedron 33:2725 (1977); Eliel, E. L. Stereochemistry of Carbon Compounds (McGraw-Hill, NY, 1962); Wilen, S. H. Tables of Resolving Agents and Optical Resolutions p. 268 (E. L. Eliel, Ed., Univ. of Notre Dame Press, Notre Dame, IN 1972), each of which is incorporated herein by reference in their entireties. It is also understood that the compounds described herein include all possible regioisomers, and mixtures thereof, which can be obtained in pure form by standard separation procedures known to those skilled in the art, and include, but are not limited to, column chromatography, thin-layer chromatography, and high-performance liquid chromatography.

Unless specifically defined, compounds provided herein can also include all isotopes of atoms occurring in the intermediates or final compounds. Isotopes include those atoms having the same atomic number but different mass numbers. Unless otherwise stated, when an atom is designated as an isotope or radioisotope (e.g., deuterium, [¹¹C], [¹⁸F]), the atom is understood to comprise the isotope or radioisotope in an amount at least greater than the natural abundance of the isotope or radioisotope. For example, when an atom is designated as “D” or “deuterium”, the position is understood to have deuterium at an abundance that is at least 3000 times greater than the natural abundance of deuterium, which is 0.015% (i.e., at least 45% incorporation of deuterium).

All compounds, and pharmaceutically acceptable salts thereof, can be found together with other substances such as water and solvents (e.g. hydrates and solvates) or can be isolated.

In some embodiments, preparation of compounds can involve the addition of acids or bases to affect, for example, catalysis of a desired reaction or formation of salt forms such as acid addition salts.

Example acids can be inorganic or organic acids and include, but are not limited to, strong and weak acids. Some example acids include hydrochloric acid, hydrobromic acid, sulfuric acid, phosphoric acid, p-toluenesulfonic acid, 4-nitrobenzoic acid, methanesulfonic acid, benzenesulfonic acid, trifluoroacetic acid, and nitric acid. Some weak acids include, but are not limited to acetic acid, propionic acid, butanoic acid, benzoic acid, tartaric acid, pentanoic acid, hexanoic acid, heptanoic acid, octanoic acid, nonanoic acid, and decanoic acid.

Example bases include lithium hydroxide, sodium hydroxide, potassium hydroxide, lithium carbonate, sodium carbonate, potassium carbonate, and sodium bicarbonate. Some example strong bases include, but are not limited to, hydroxide, alkoxides, metal amides, metal hydrides, metal dialkylamides and arylamines, wherein; alkoxides include lithium, sodium and potassium salts of methyl, ethyl and t-butyl oxides; metal amides include sodium amide, potassium amide and lithium amide; metal hydrides include sodium hydride, potassium hydride and lithium hydride; and metal dialkylamides include lithium, sodium, and potassium salts of methyl, ethyl, n-propyl, iso-propyl, n-butyl, tert-butyl, trimethylsilyl and cyclohexyl substituted amides.

In some embodiments, the compounds provided herein, or salts thereof, are substantially isolated. By “substantially isolated” is meant that the compound is at least partially or substantially separated from the environment in which it was formed or detected. Partial separation can include, for example, a composition enriched in the compounds provided herein. Substantial separation can include compositions containing at least about 50%, at least about 60%, at least about 70%, at least about 80%, at least about 90%, at least about 95%, at least about 97%, or at least about 99% by weight of the compounds provided herein, or salt thereof. Methods for isolating compounds and their salts are routine in the art.

The expressions, “ambient temperature” and “room temperature” or “rt” as used herein, are understood in the art, and refer generally to a temperature, e.g. a reaction temperature, that is about the temperature of the room in which the reaction is carried out, for example, a temperature from about 20 °C to about 30 °C.

The present application also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present application include the conventional non-toxic salts of the parent compound formed, for example, from non-toxic inorganic or organic acids. The pharmaceutically acceptable salts of the present application can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (MeCN) are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977). Conventional methods for preparing salt forms are described, for example, in Handbook of Pharmaceutical Salts: Properties, Selection, and Use, Wiley-VCH, 2002

The phrase “small molecule,” as used herein, refers to molecules, in particular active agents, having a molecular weight of less than 1000 Da (e.g., less than 800 Da, or less than 500 Da).

The phrase ‘biologic drug’ includes any therapeutic agent, whether in discovery, formulation, or commercial production, derived from natural biological sources or synthesized to resemble or be equivalent to biological products. Biologic drugs include, but are not limited to, proteins of any sort, including monoclonal antibodies, polyclonal antibodies, polysaccharides, protein-polysaccharide complexes, oligopeptides, vaccines, lipid nanoparticles, viral capsids, nucleic acids, oligonucleotides, and polynucleic acids such as RNA and DNA. Also included are agents, not necessarily biological, which are used to enhance, functionalize, encapsulate, or deliver a biological agent, such as, but not limited to, encapsulation/delivery synthetic polymers, dendrimers, liposomes, vesicles, and micelles.

### IWS1 Inhibitors

The methods described herein can employ IWS1 inhibitors. IWS1 is a component of the RNA polymerase II Elongation Complex (RNAPII EC). IWS1 (which stands for “Interacts with Spt6 Protein 1) is an essential component of the RNAPII EC. Cryo-electron microscopy structures of working RNAPII ECs suggest that IWS1 physically interacts with Spt6, Spt5, and the Clamp domain of RNAPII.

Any suitable IWS1 inhibitor can be used in connection with the methods described herein. The IWS1 inhibitor can comprise a small molecule inhibitor or, for example, a biologic drug that inhibits IWS1. In certain embodiments, IWS1 inhibitor can comprise a small molecule inhibitor of IWS1.

In some embodiments, the IWS1 inhibitor can target an IWS1/Spt6 interface. In some embodiments, the IWS1 inhibitor can reduce the binding of IWS1 and Spt6. In some embodiments, the IWS1 inhibitor reduces the formation of the RNAPII complex. In some embodiments, the IWS1 inhibitor down-regulates the transcriptional activities of RNAPII.

In certain embodiments, the IWS1 inhibitor comprises a tricyclic fused ring system.

In some embodiments, the IWS1 inhibitor is defined by Formula I or Formula II

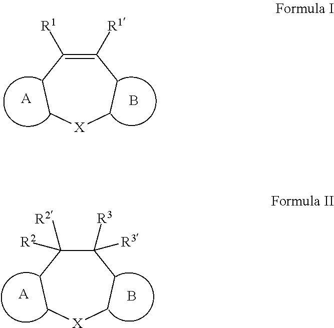

or a pharmaceutically acceptable salt or prodrug thereof, wherein
   X is NR⁴, CR⁵R^{5′}, or —C═CR⁶R^{6′};
   A and B are each individually selected from a fused 3-10 membered monocyclic or bicyclic cycloalkyl ring, a fused 3-10 membered monocyclic or bicyclic hecycloalkyl ring, a fused 5-11 membered monocyclic or bicyclic aryl ring, and a fused 5-11 membered monocyclic or bicyclic heteroaryl ring, each optionally substituted by 1, 2, 3, or 4 independently selected R^{A} groups;
   R¹ and R^{1′} are individually selected from H, halo, OH, OR^{B}, CN, NO₂, C₁₋₄ alkyl, C₁₋₄ haloalkyl, C₂₋₄ alkenyl, or C₂₋₄ alkynyl, wherein the C₁₋₄ alkyl, C₁₋₄ haloalkyl, C₂₋₄ alkenyl, and C₂₋₄ alkynyl groups are each optionally substituted by 1, 2, 3, or 4 independently selected R^{A} groups;
   R² and R^{2′} are individually selected from H, halo, OH, OR^{B}, CN, NO₂, C₁₋₄ alkyl, C₁₋₄ haloalkyl, C₂₋₄ alkenyl, or C₂₋₄ alkynyl, wherein the C₁₋₄ alkyl, C₁₋₄ haloalkyl, C₂₋₄ alkenyl, and C₂₋₄ alkynyl groups are each optionally substituted by 1, 2, 3, or 4 independently selected R^{A} groups; or wherein R² and R^{2′} together form a ketone group;
   R³ and R^{3′} are individually selected from H, halo, OH, OR^{B}, CN, NO₂, C₁₋₄ alkyl, C₁₋₄ haloalkyl, C₂₋₄ alkenyl, or C₂₋₄ alkynyl, wherein the C₁₋₄ alkyl, C₁₋₄ haloalkyl, C₂₋₄ alkenyl, and C₂₋₄ alkynyl groups are each optionally substituted by 1, 2, 3, or 4 independently selected R^{A} groups; or wherein R³ and R^{3′} together form a ketone group;
   R⁴ is selected from hydrogen, C₁₋₆ alkyl, C₂₋₆ alkenyl, C₂₋₆ alkynyl, C₁₋₄ haloalkyl, C₁₋₆ alkoxy, C₁₋₆ haloalkoxy, a 3-10 membered monocyclic or bicyclic cycloalkyl ring, a 3-10 membered monocyclic or bicyclic hecycloalkyl ring, a 5-11 membered monocyclic or bicyclic aryl ring, a 5-11 membered monocyclic or bicyclic heteroaryl ring, cyano-C₁₋₃ alkyl, HO—C₁₋₃ alkyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino, each optionally substituted by 1, 2, 3, or 4 independently selected R^{A} groups;
   R⁵ and R^{5′} are individually selected from H, halo, OH, OR^{B}, CN, NO₂, C₁₋₆ alkyl, C₂₋₆ alkenyl, C₂₋₆ alkynyl, C₁₋₄ haloalkyl, C₁₋₆ alkoxy, C₁₋₆ haloalkoxy, a 3-10 membered monocyclic or bicyclic cycloalkyl ring, a 3-10 membered monocyclic or bicyclic hecycloalkyl ring, a 5-11 membered monocyclic or bicyclic aryl ring, a 5-11 membered monocyclic or bicyclic heteroaryl ring, each optionally substituted by 1, 2, 3, or 4 independently selected R^{A} groups; or wherein R⁵ and R^{5′} together form a ketone group;
   R⁶ and R^{6′} are individually selected from H, halo, OH, OR^{B}, CN, NO₂, C₁₋₆ alkyl, C₂₋₆ alkenyl, C₂₋₆ alkynyl, C₁₋₄ haloalkyl, C₁₋₆ alkoxy, C₁₋₆ haloalkoxy, a 3-10 membered monocyclic or bicyclic cycloalkyl ring, a 3-10 membered monocyclic or bicyclic hecycloalkyl ring, a 5-11 membered monocyclic or bicyclic aryl ring, a 5-11 membered monocyclic or bicyclic heteroaryl ring, each optionally substituted by 1, 2, 3, or 4 independently selected R^{A} groups;
   each R^{A} is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino; and
   R^{B} is selected from hydrogen, C₁₋₆ alkyl, C₁₋₄haloalkyl, C₂₋₆ alkenyl, C₂₋₆ alkynyl, C₃₋₁₀ cycloalkyl, and 6-10 membered aryl, wherein the C₁₋₆ alkyl, C₁₋₄haloalkyl, C₂₋₆ alkenyl, C₂₋₆ alkynyl, C₃₋₁₀ cycloalkyl, and 6-10 membered aryl are each optionally substituted by 1, 2, 3, or 4 independently selected R^{A} groups.

In some of these embodiments, A is a phenyl ring. In other embodiments, A is a pyridyl ring. In other embodiments, A is a 5-membered heterocyclic ring chosen from a thiophene ring, a pyrrolidine ring, a pyrroline ring, a pyrrole ring, a pyrazolidine ring, an imidazolidine ring, a pyrazoline ring, and imidazoline ring, a pyrazole ring, a imidazole ring, a triazole ring, a furyl ring, a dioxolane ring, a thiophenyl ring, an oxazole ring, an isoxazole ring, an isothiazole ring, a thiazole ring, an oxathiolane ring, oxadiazole ring, or a thiadiazole ring.

In some of these embodiments, B is a phenyl ring. In other embodiments, B is a pyridyl ring. In other embodiments, B is a 5-membered heterocyclic ring chosen from a thiophene ring, a pyrrolidine ring, a pyrroline ring, a pyrrole ring, a pyrazolidine ring, an imidazolidine ring, a pyrazoline ring, and imidazoline ring, a pyrazole ring, a imidazole ring, a triazole ring, a furyl ring, a dioxolane ring, a thiophenyl ring, an oxazole ring, an isoxazole ring, an isothiazole ring, a thiazole ring, an oxathiolane ring, oxadiazole ring, or a thiadiazole ring.

In certain of these embodiments, A is a phenyl ring and B is a phenyl ring. In other of these embodiments, A is a phenyl ring and B is a pyridyl ring. In other of these embodiments, A is a phenyl ring and B is a thiophene ring.

In some embodiments, the IWS1 inhibitor is defined by Formula IA or Formula IIA

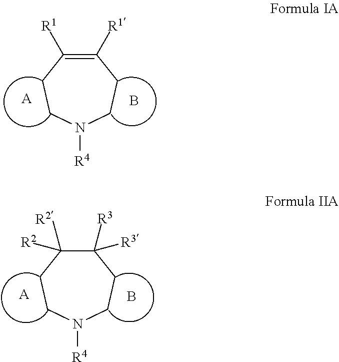

or a pharmaceutically acceptable salt or prodrug thereof, wherein
   A and B are each individually selected from a fused 3-10 membered monocyclic or bicyclic cycloalkyl ring, a fused 3-10 membered monocyclic or bicyclic hecycloalkyl ring, a fused 5-11 membered monocyclic or bicyclic aryl ring, and a fused 5-11 membered monocyclic or bicyclic heteroaryl ring, each optionally substituted by 1, 2, 3, or 4 independently selected R^{A} groups;
   R¹ and R^{1′} are individually selected from H, halo, OH, OR^{B}, CN, NO₂, C₁₋₄ alkyl, C₁₋₄ haloalkyl, C₂₋₄ alkenyl, or C₂₋₄ alkynyl, wherein the C₁₋₄ alkyl, C₁₋₄ haloalkyl, C₂₋₄ alkenyl, and C₂₋₄ alkynyl groups are each optionally substituted by 1, 2, 3, or 4 independently selected R^{A} groups;
   R² and R^{2′} are individually selected from H, halo, OH, OR^{B}, CN, NO₂, C₁₋₄ alkyl, C₁₋₄ haloalkyl, C₂₋₄ alkenyl, or C₂₋₄ alkynyl, wherein the C₁₋₄ alkyl, C₁₋₄ haloalkyl, C₂₋₄ alkenyl, and C₂₋₄ alkynyl groups are each optionally substituted by 1, 2, 3, or 4 independently selected R^{A} groups; or wherein R² and R^{2′} together form a ketone group;
   R³ and R^{3′} are individually selected from H, halo, OH, OR^{B}, CN, NO₂, C₁₋₄ alkyl, C₁₋₄ haloalkyl, C₂₋₄ alkenyl, or C₂₋₄ alkynyl, wherein the C₁₋₄ alkyl, C₁₋₄ haloalkyl, C₂₋₄ alkenyl, and C₂₋₄ alkynyl groups are each optionally substituted by 1, 2, 3, or 4 independently selected R^{A} groups; or wherein R³ and R^{3′} together form a ketone group;
   R⁴ is selected from hydrogen, C₁₋₆ alkyl, C₂₋₆ alkenyl, C₂₋₆ alkynyl, C₁₋₄ haloalkyl, C₁₋₆ alkoxy, C₁₋₆ haloalkoxy, a 3-10 membered monocyclic or bicyclic cycloalkyl ring, a 3-10 membered monocyclic or bicyclic hecycloalkyl ring, a 5-11 membered monocyclic or bicyclic aryl ring, a 5-11 membered monocyclic or bicyclic heteroaryl ring, cyano-C₁₋₃ alkyl, HO—C₁₋₃ alkyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino, each optionally substituted by 1, 2, 3, or 4 independently selected R^{A} groups;
   each R^{A} is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino; and
   R^{B} is selected from hydrogen, C₁₋₆ alkyl, C₁₋₄haloalkyl, C₂₋₆ alkenyl, C₂₋₆ alkynyl, C₃₋₁₀ cycloalkyl, and 6-10 membered aryl, wherein the C₁₋₆ alkyl, C₁₋₄haloalkyl, C₂₋₆ alkenyl, C₂₋₆ alkynyl, C₃₋₁₀ cycloalkyl, and 6-10 membered aryl are each optionally substituted by 1, 2, 3, or 4 independently selected R^{A} groups.

In some of these embodiments, A is a phenyl ring. In other embodiments, A is a pyridyl ring. In other embodiments, A is a 5-membered heterocyclic ring chosen from a thiophene ring, a pyrrolidine ring, a pyrroline ring, a pyrrole ring, a pyrazolidine ring, an imidazolidine ring, a pyrazoline ring, and imidazoline ring, a pyrazole ring, a imidazole ring, a triazole ring, a furyl ring, a dioxolane ring, a thiophenyl ring, an oxazole ring, an isoxazole ring, an isothiazole ring, a thiazole ring, an oxathiolane ring, oxadiazole ring, or a thiadiazole ring.

In some of these embodiments, B is a phenyl ring. In other embodiments, B is a pyridyl ring. In other embodiments, B is a 5-membered heterocyclic ring chosen from a thiophene ring, a pyrrolidine ring, a pyrroline ring, a pyrrole ring, a pyrazolidine ring, an imidazolidine ring, a pyrazoline ring, and imidazoline ring, a pyrazole ring, a imidazole ring, a triazole ring, a furyl ring, a dioxolane ring, a thiophenyl ring, an oxazole ring, an isoxazole ring, an isothiazole ring, a thiazole ring, an oxathiolane ring, oxadiazole ring, or a thiadiazole ring.

In certain of these embodiments, A is a phenyl ring and B is a phenyl ring. In other of these embodiments, A is a phenyl ring and B is a pyridyl ring. In other of these embodiments, A is a phenyl ring and B is a thiophene ring.

In some of these embodiments, R⁴ is C₁₋₆ alkyl optionally substituted by 1, 2, 3, or 4 independently selected R^{A} groups.

In some embodiments, the IWS1 inhibitor is defined by Formula IB or Formula IIB

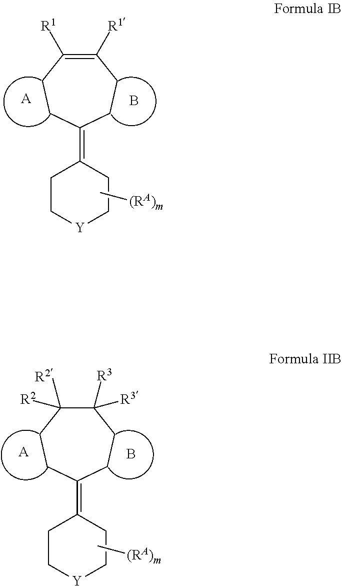

or a pharmaceutically acceptable salt or prodrug thereof, wherein
   Y is NR⁴ or CR⁵R^{5′};
   A and B are each individually selected from a fused 3-10 membered monocyclic or bicyclic cycloalkyl ring, a fused 3-10 membered monocyclic or bicyclic hecycloalkyl ring, a fused 5-11 membered monocyclic or bicyclic aryl ring, and a fused 5-11 membered monocyclic or bicyclic heteroaryl ring, each optionally substituted by 1, 2, 3, or 4 independently selected R^{A} groups;
   R¹ and R^{1′} are individually selected from H, halo, OH, OR^{B}, CN, NO₂, C₁₋₄ alkyl, C₁₋₄ haloalkyl, C₂₋₄ alkenyl, or C₂₋₄ alkynyl, wherein the C₁₋₄ alkyl, C₁₋₄ haloalkyl, C₂₋₄ alkenyl, and C₂₋₄ alkynyl groups are each optionally substituted by 1, 2, 3, or 4 independently selected R^{A} groups;
   R² and R^{2′} are individually selected from H, halo, OH, OR^{B}, CN, NO₂, C₁₋₄ alkyl, C₁₋₄ haloalkyl, C₂₋₄ alkenyl, or C₂₋₄ alkynyl, wherein the C₁₋₄ alkyl, C₁₋₄ haloalkyl, C₂₋₄ alkenyl, and C₂₋₄ alkynyl groups are each optionally substituted by 1, 2, 3, or 4 independently selected R^{A} groups; or wherein R² and R^{2′} together form a ketone group;
   R³ and R^{3′} are individually selected from H, halo, OH, OR^{B}, CN, NO₂, C₁₋₄ alkyl, C₁₋₄ haloalkyl, C₂₋₄ alkenyl, or C₂₋₄ alkynyl, wherein the C₁₋₄ alkyl, C₁₋₄ haloalkyl, C₂₋₄ alkenyl, and C₂₋₄ alkynyl groups are each optionally substituted by 1, 2, 3, or 4 independently selected R^{A} groups; or wherein R³ and R^{3′} together form a ketone group;
   R⁴ is selected from hydrogen, C₁₋₆ alkyl, C₂₋₆ alkenyl, C₂₋₆ alkynyl, C₁₋₄ haloalkyl, C₁₋₆ alkoxy, C₁₋₆ haloalkoxy, a 3-10 membered monocyclic or bicyclic cycloalkyl ring, a 3-10 membered monocyclic or bicyclic hecycloalkyl ring, a 5-11 membered monocyclic or bicyclic aryl ring, a 5-11 membered monocyclic or bicyclic heteroaryl ring, cyano-C₁₋₃ alkyl, HO—C₁₋₃ alkyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino, each optionally substituted by 1, 2, 3, or 4 independently selected R^{A} groups;
   R⁵ and R^{5′} are individually selected from H, halo, OH, OR^{B}, CN, NO₂, C₁₋₆ alkyl, C₂₋₆ alkenyl, C₂₋₆ alkynyl, C₁₋₄ haloalkyl, C₁₋₆ alkoxy, C₁₋₆ haloalkoxy, a 3-10 membered monocyclic or bicyclic cycloalkyl ring, a 3-10 membered monocyclic or bicyclic hecycloalkyl ring, a 5-11 membered monocyclic or bicyclic aryl ring, a 5-11 membered monocyclic or bicyclic heteroaryl ring, each optionally substituted by 1, 2, 3, or 4 independently selected R^{A} groups;
   each R^{A} is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino;
   m is an integer from 0 to 4; and
   R^{B} is selected from hydrogen, C₁₋₆ alkyl, C₁₋₄haloalkyl, C₂₋₆ alkenyl, C₂₋₆ alkynyl, C₃₋₁₀ cycloalkyl, and 6-10 membered aryl, wherein the C₁₋₆ alkyl, C₁₋₄haloalkyl, C₂₋₆ alkenyl, C₂₋₆ alkynyl, C₃₋₁₀ cycloalkyl, and 6-10 membered aryl are each optionally substituted by 1, 2, 3, or 4 independently selected R^{A} groups.

In some of these embodiments, A is a phenyl ring. In other embodiments, A is a pyridyl ring. In other embodiments, A is a 5-membered heterocyclic ring chosen from a thiophene ring, a pyrrolidine ring, a pyrroline ring, a pyrrole ring, a pyrazolidine ring, an imidazolidine ring, a pyrazoline ring, and imidazoline ring, a pyrazole ring, a imidazole ring, a triazole ring, a furyl ring, a dioxolane ring, a thiophenyl ring, an oxazole ring, an isoxazole ring, an isothiazole ring, a thiazole ring, an oxathiolane ring, oxadiazole ring, or a thiadiazole ring.

In some of these embodiments, B is a phenyl ring. In other embodiments, B is a pyridyl ring. In other embodiments, B is a 5-membered heterocyclic ring chosen from a thiophene ring, a pyrrolidine ring, a pyrroline ring, a pyrrole ring, a pyrazolidine ring, an imidazolidine ring, a pyrazoline ring, and imidazoline ring, a pyrazole ring, a imidazole ring, a triazole ring, a furyl ring, a dioxolane ring, a thiophenyl ring, an oxazole ring, an isoxazole ring, an isothiazole ring, a thiazole ring, an oxathiolane ring, oxadiazole ring, or a thiadiazole ring.

In certain of these embodiments, A is a phenyl ring and B is a phenyl ring. In other of these embodiments, A is a phenyl ring and B is a pyridyl ring. In other of these embodiments, A is a phenyl ring and B is a thiophene ring.

In some of these embodiments, Y is NR⁴. In certain of these embodiments, R⁴ is selected from hydrogen and C₁₋₆ alkyl.

In some of these embodiments, m is 0.

In some examples, the IWS1 inhibitor comprises a compound shown below

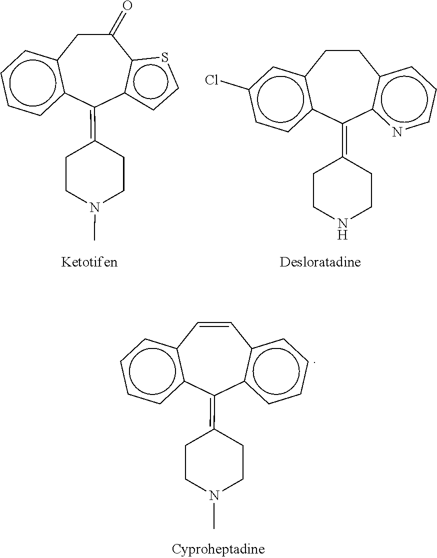

In some embodiments, IWS1 inhibitor can comprise ketotifen or a pharmaceutically effective salt or prodrug thereof. In some embodiments, IWS1 inhibitor can comprise ketotifen or a ketotifen derivative (i.e., a compound sharing the same tricyclic ring structure as ketotifen). In certain embodiments, IWS1 inhibitor can comprise ketotifen.

In some embodiments, IWS1 inhibitor can comprise desloratadine or a pharmaceutically effective salt or prodrug thereof. In some embodiments, IWS1 inhibitor can comprise desloratadine or a desloratadine derivative (i.e., a compound sharing the same tricyclic ring structure as desloratadine). In certain embodiments, IWS1 inhibitor can comprise desloratadine.

In some embodiments, IWS1 inhibitor can comprise cyproheptadine or a pharmaceutically effective salt or prodrug thereof. In some embodiments, IWS1 inhibitor can comprise cyproheptadine or a cyproheptadine derivative (i.e., a compound sharing the same tricyclic ring structure as cyproheptadine). In certain embodiments, IWS1 inhibitor can comprise cyproheptadine.

In some examples, the IWS1 inhibitor comprises a compound shown below

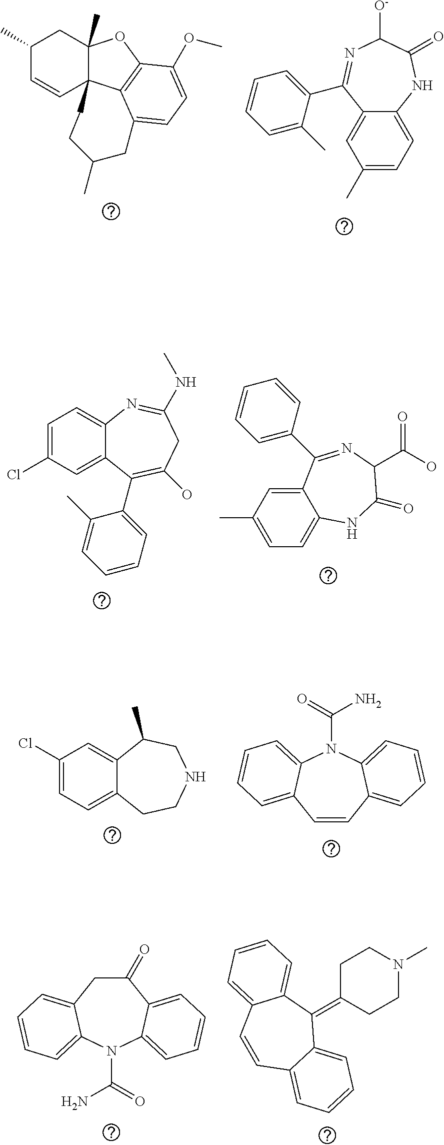

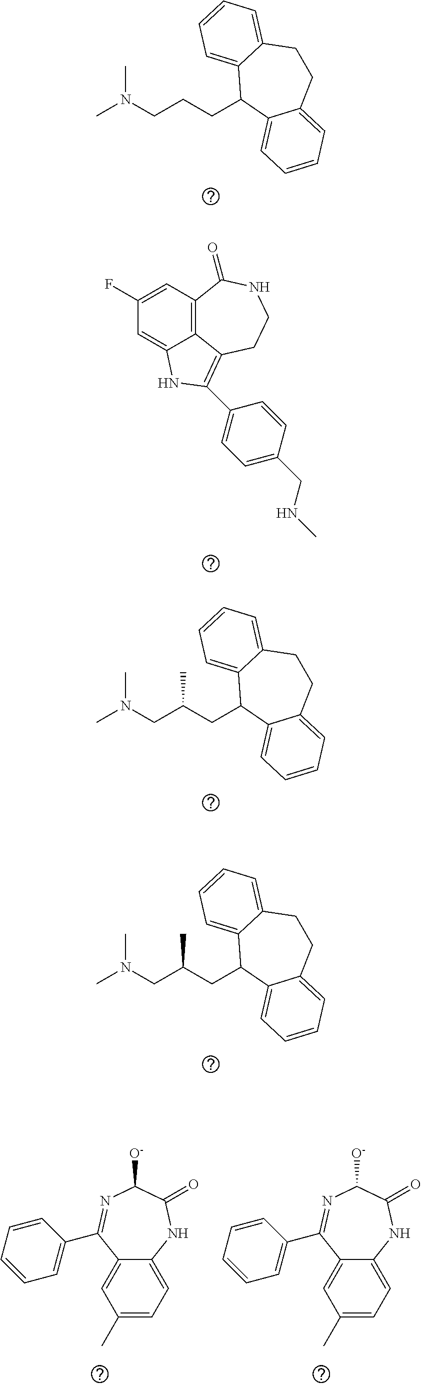 [SMILES: CN(C)CCCC1c2ccccc2CCc2ccccc21 ; CNCc1ccc(-c2[nH]c3cc(F)cc4c3c2CCNC4=O)cc1 ; C[C@H](CC1c2ccccc2CCc2ccccc21)CN(C)C ; C[C@@H](CC1c2ccccc2CCc2ccccc21)CN(C)C ; Cc1ccc2c(c1)C(c1ccccc1)=N[C@@H]([O-])C(=O)N2 ; Cc1ccc2c(c1)C(c1ccccc1)=N[C@H]([O-])C(=O)N2]

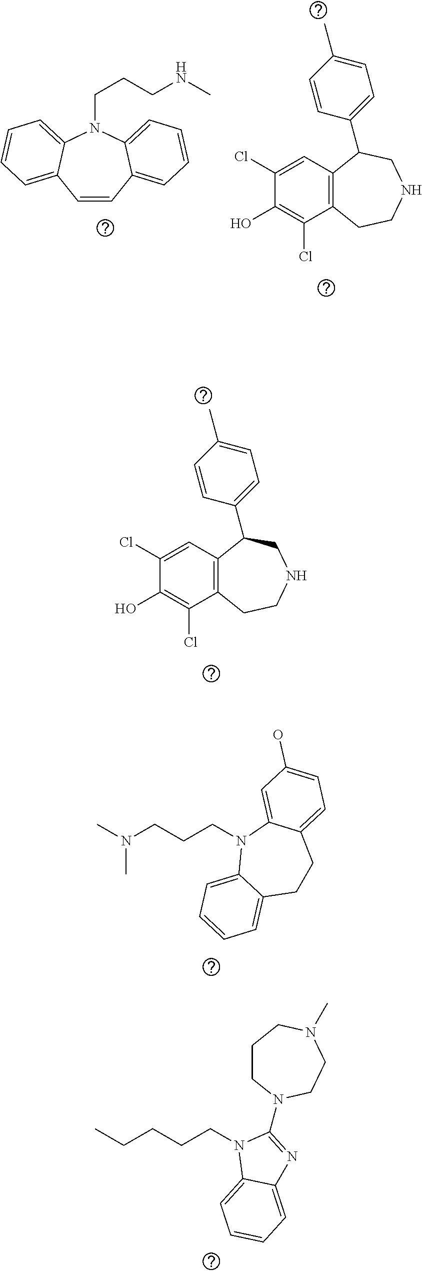 [SMILES: CNCCCN1c2ccccc2C=Cc2ccccc21 ; Cc1ccc(C2CNCCc3c2cc(Cl)c(O)c3Cl)cc1 ; Cc1ccc([C@H]2CNCCc3c2cc(Cl)c(O)c3Cl)cc1 ; CN(C)CCCN1c2ccccc2CCc2ccc(O)cc21 ; CCCCCn1c(N2CCCN(C)CC2)nc2ccccc21]

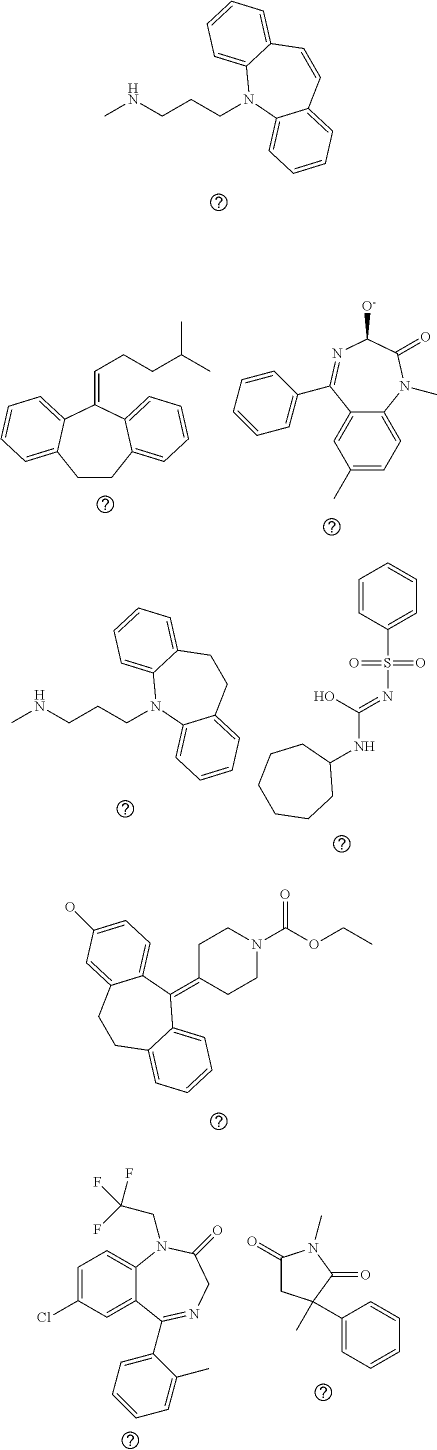

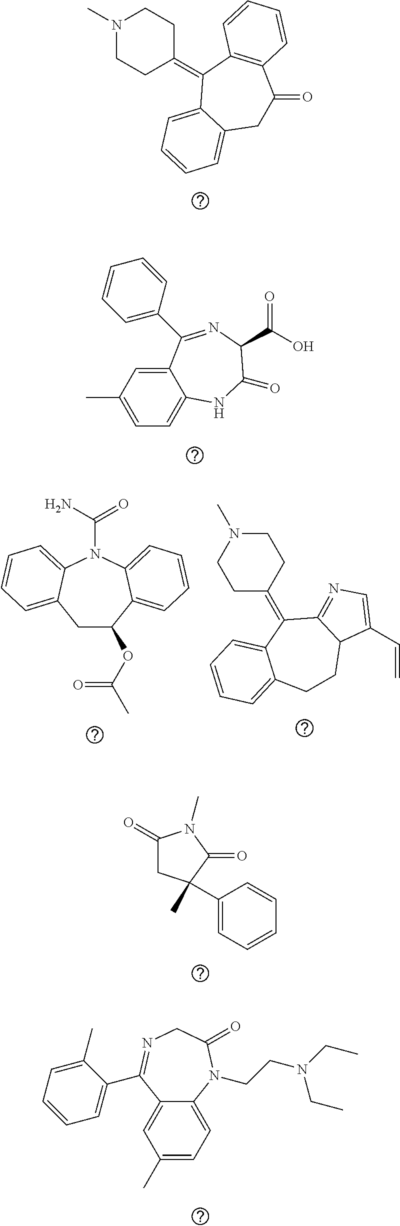

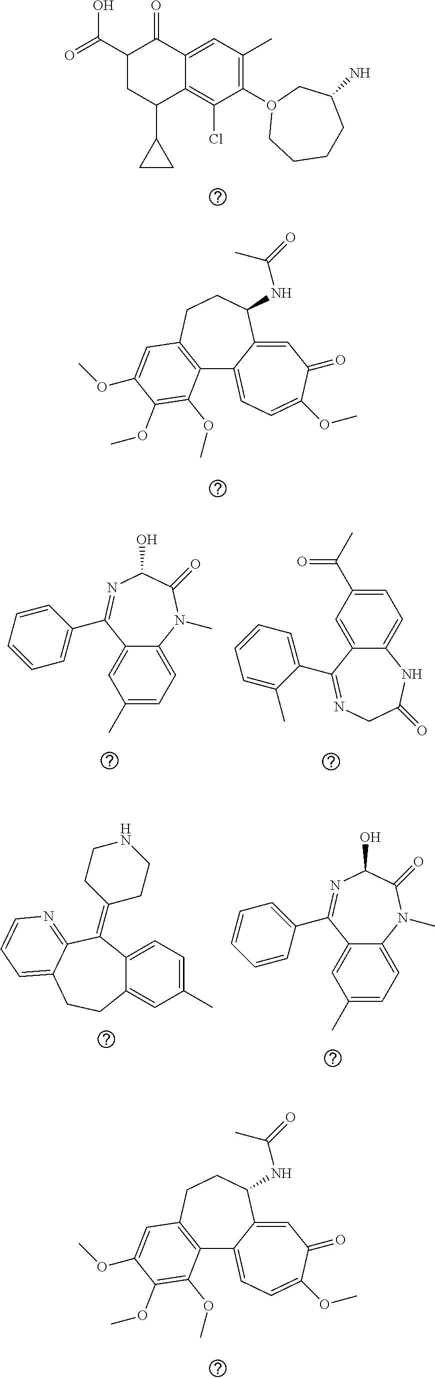

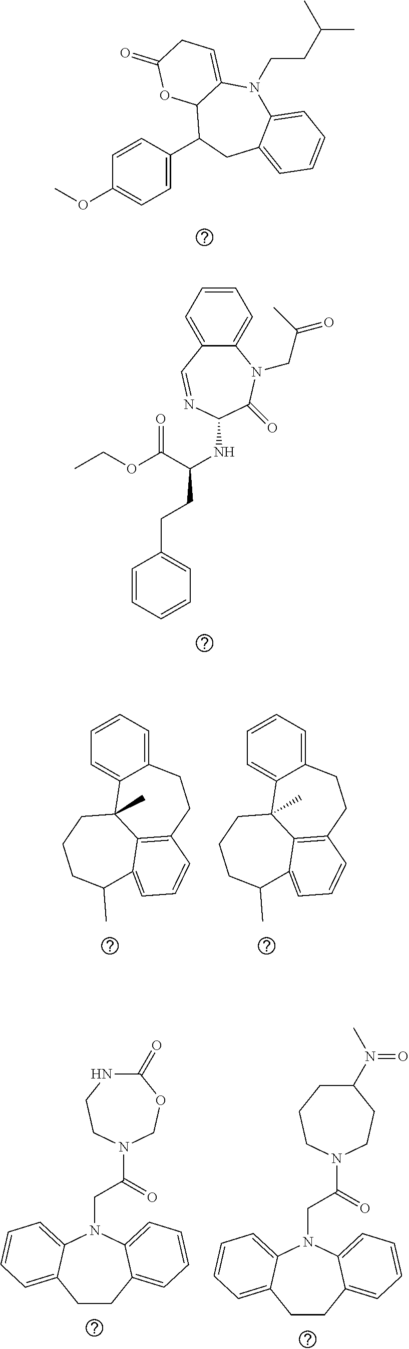

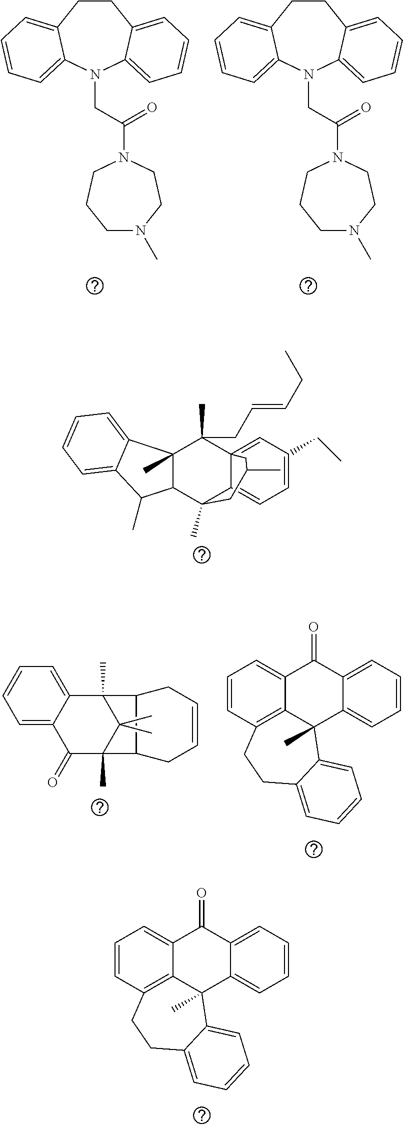

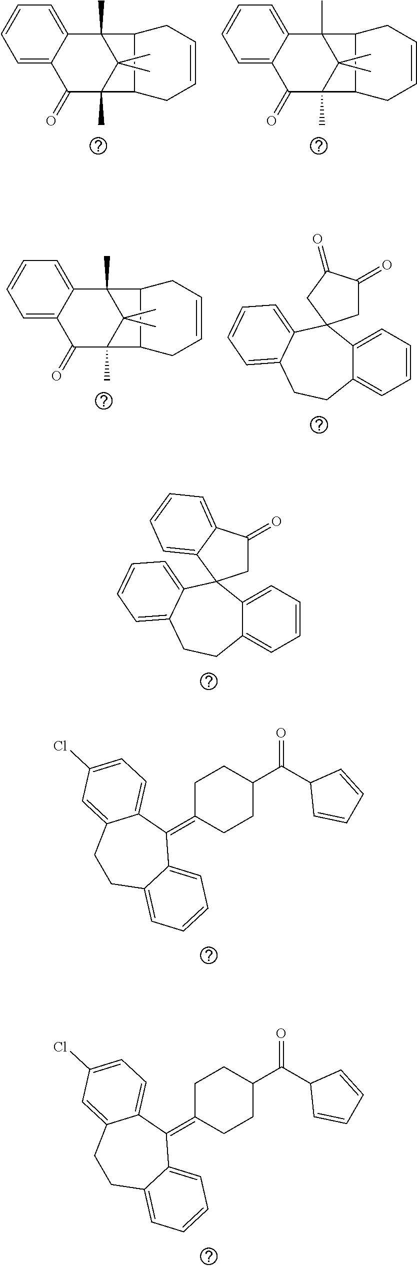

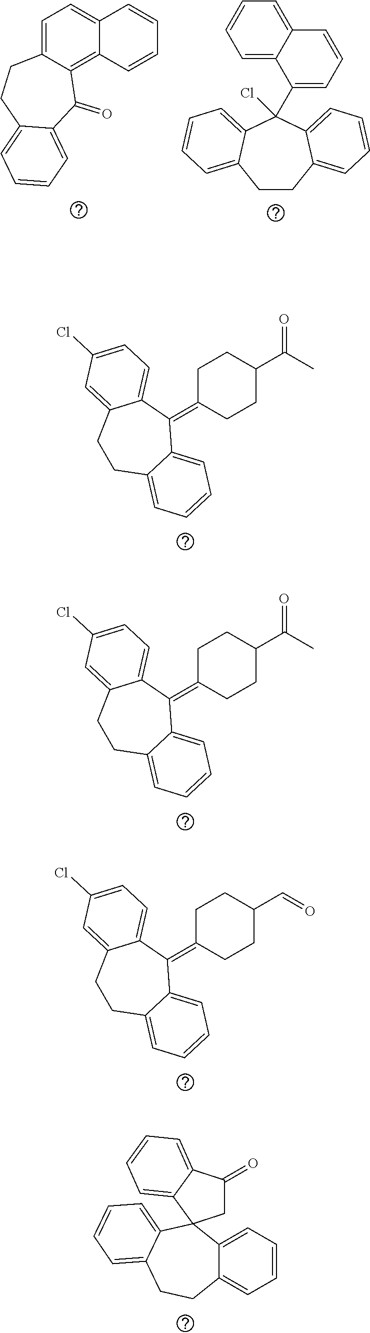 [SMILES: O=C1c2ccccc2CCc2ccc3ccccc3c21 ; ClC1(c2cccc3ccccc23)c2ccccc2CCc2ccccc21 ; CC(=O)C1CCC(=C2c3ccccc3CCc3cc(Cl)ccc32)CC1 ; CC(=O)C1CCC(=C2c3ccccc3CCc3cc(Cl)ccc32)CC1 ; O=CC1CCC(=C2c3ccccc3CCc3cc(Cl)ccc32)CC1 ; O=C1CC2(c3ccccc3CCc3ccccc32)c2ccccc21]

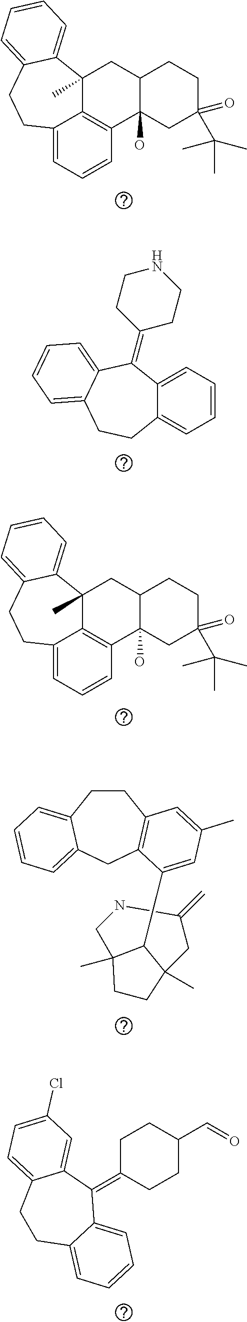

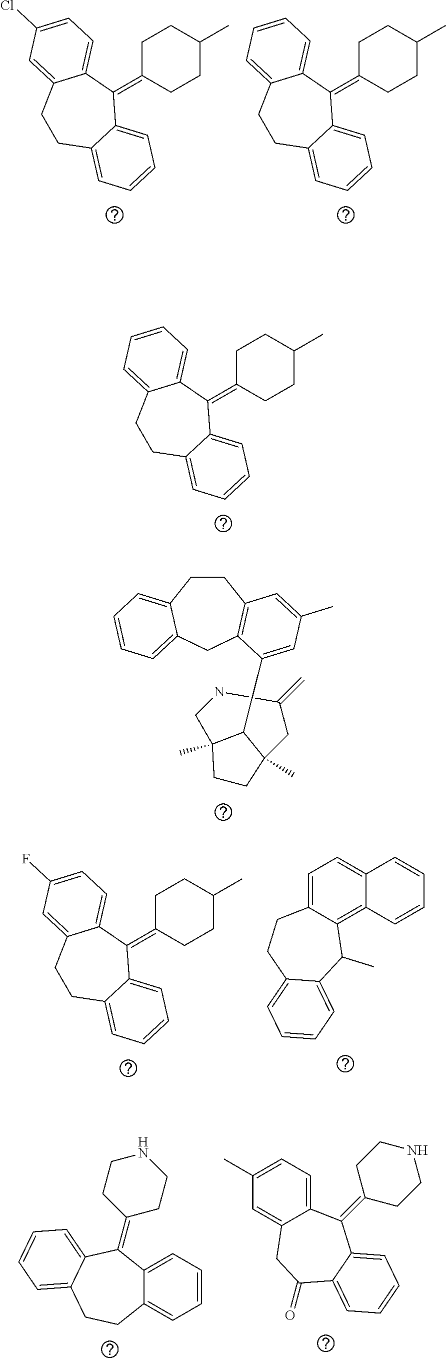

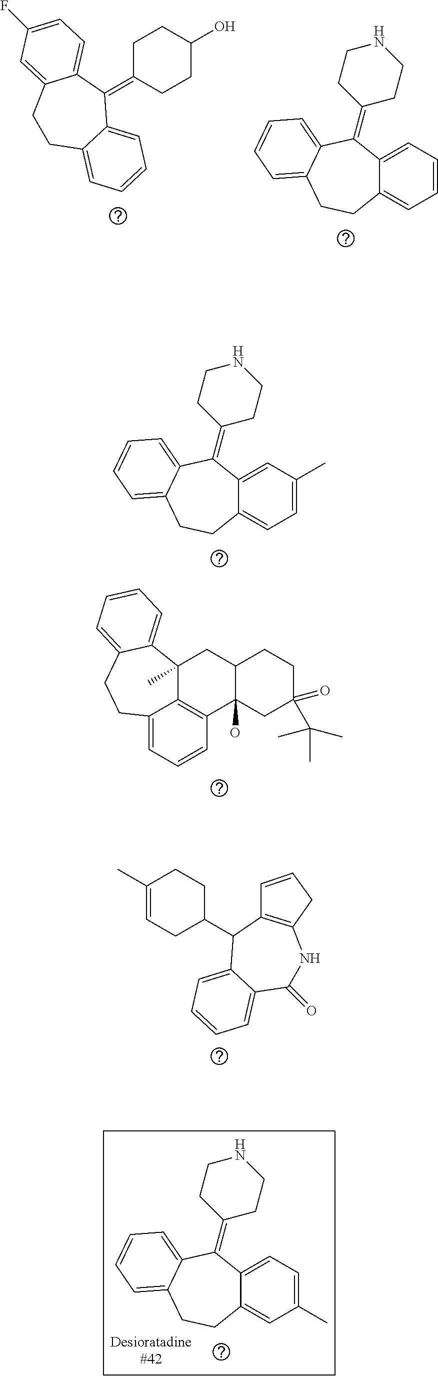

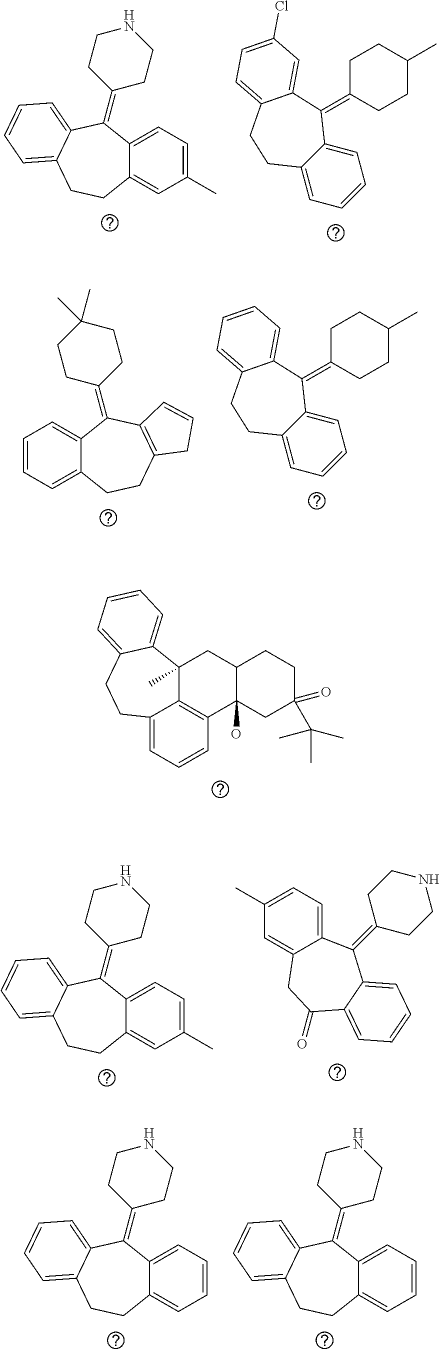

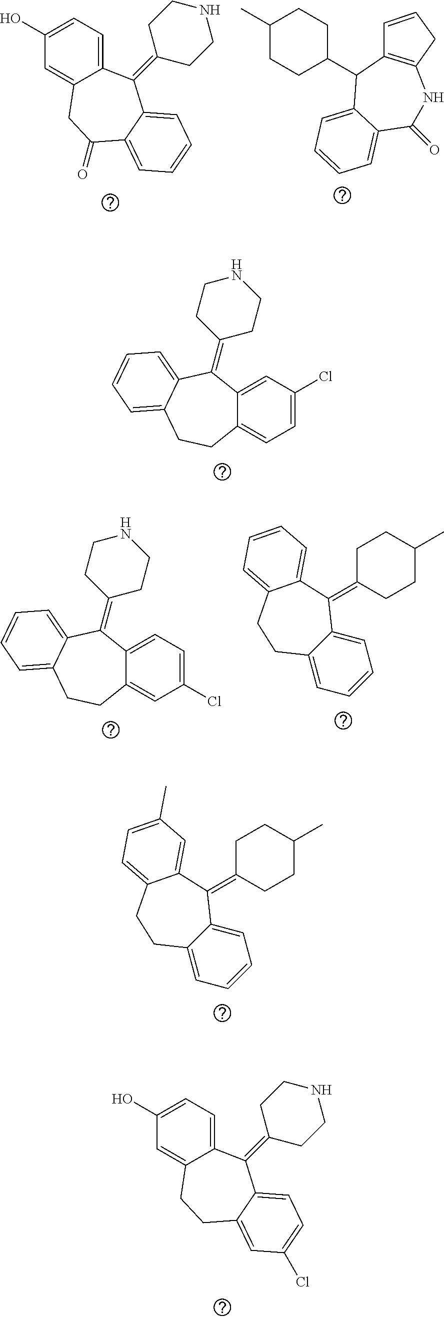

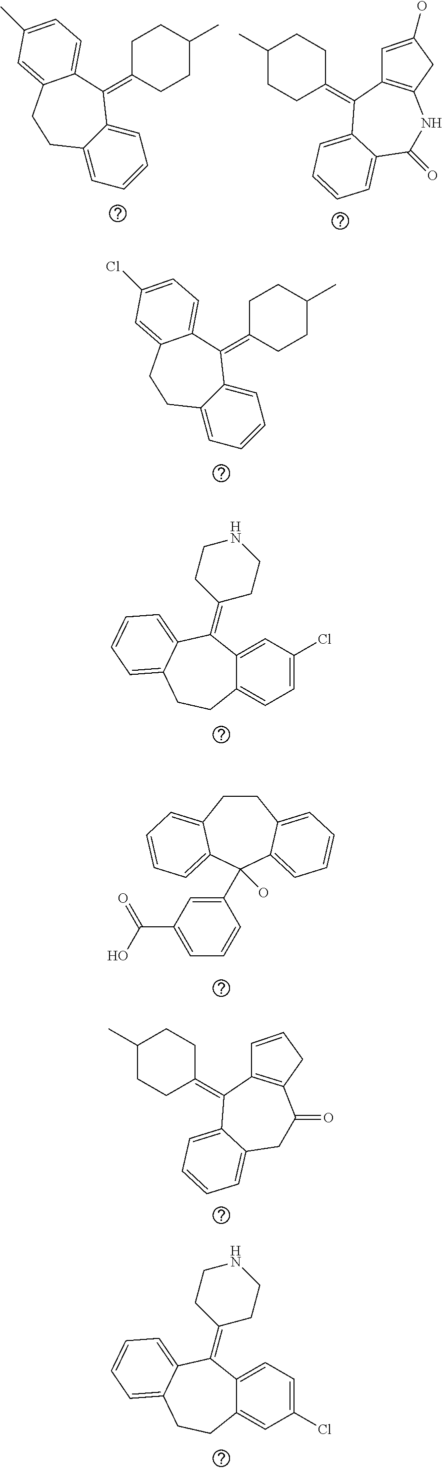

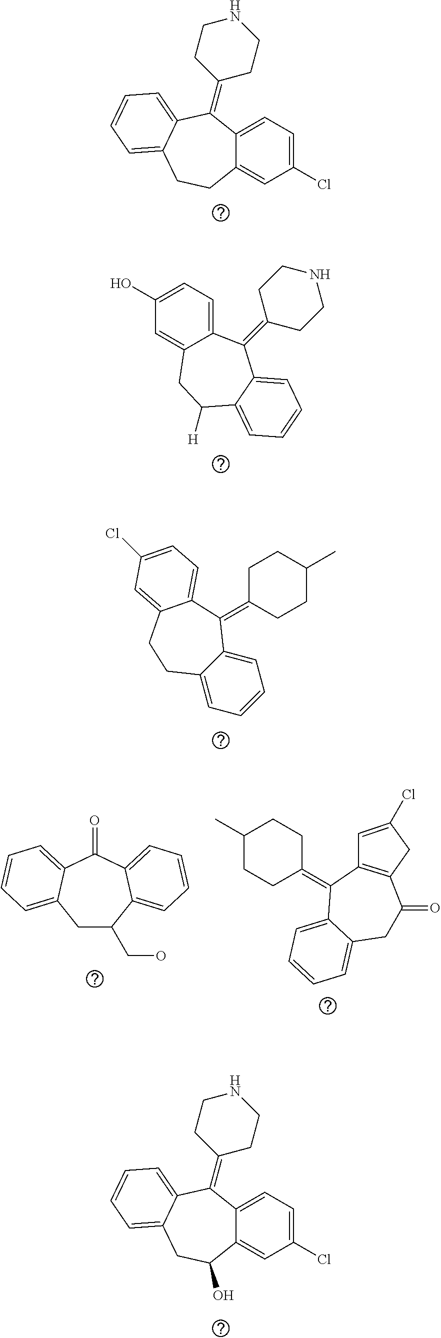 [SMILES: Clc1ccc2c(c1)CCc1ccccc1C2=C1CCNCC1 ; Oc1ccc2c(c1)CCc1ccccc1C2=C1CCNCC1 ; CC1CCC(=C2c3ccccc3CCc3cc(Cl)ccc32)CC1 ; O=C1c2ccccc2CC(CO)c2ccccc21 ; CC1CCC(=C2C3=C(CC(Cl)=C3)C(=O)Cc3ccccc32)CC1 ; O[C@H]1Cc2ccccc2C(=C2CCNCC2)c2ccc(Cl)cc21]

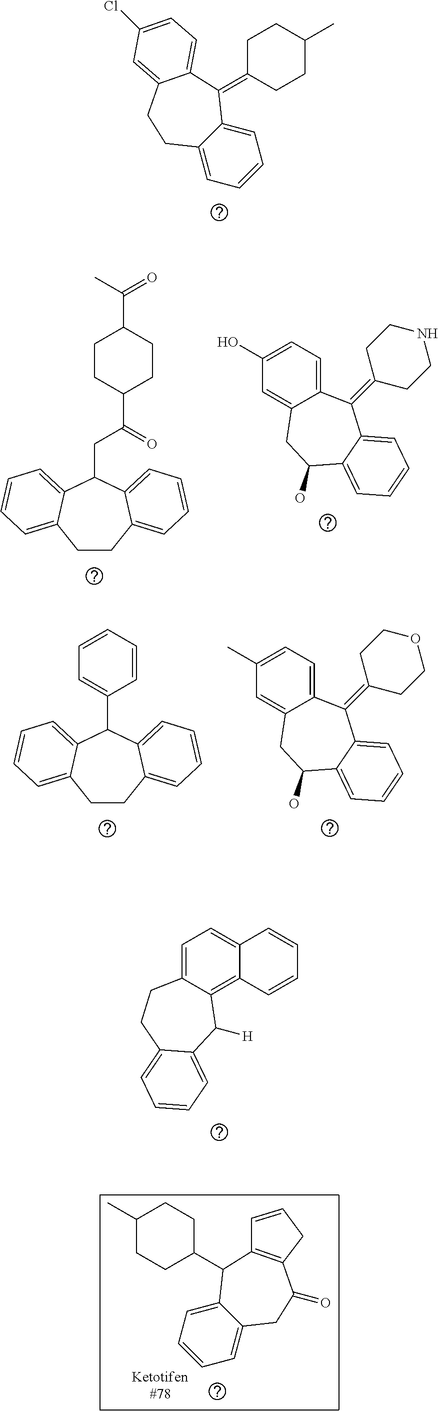

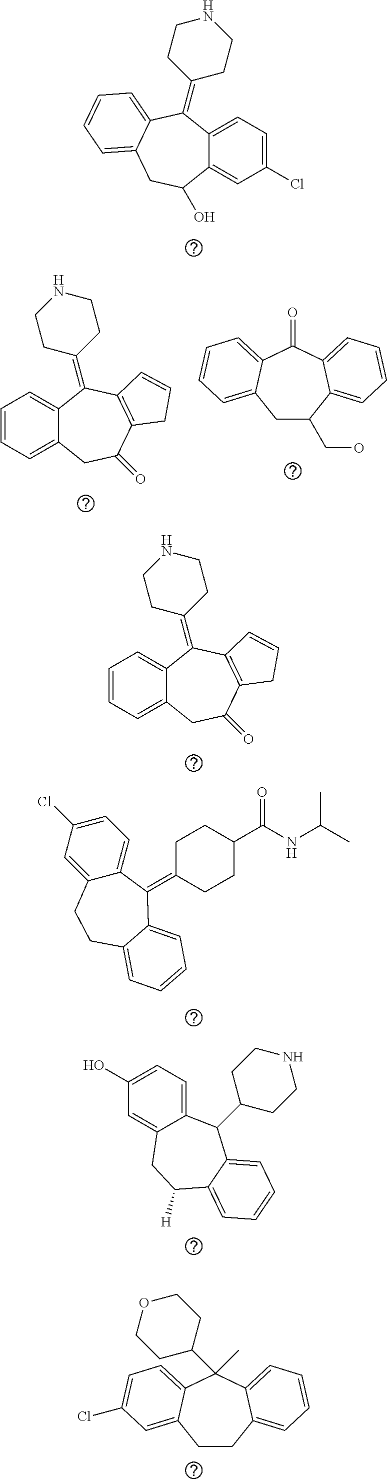

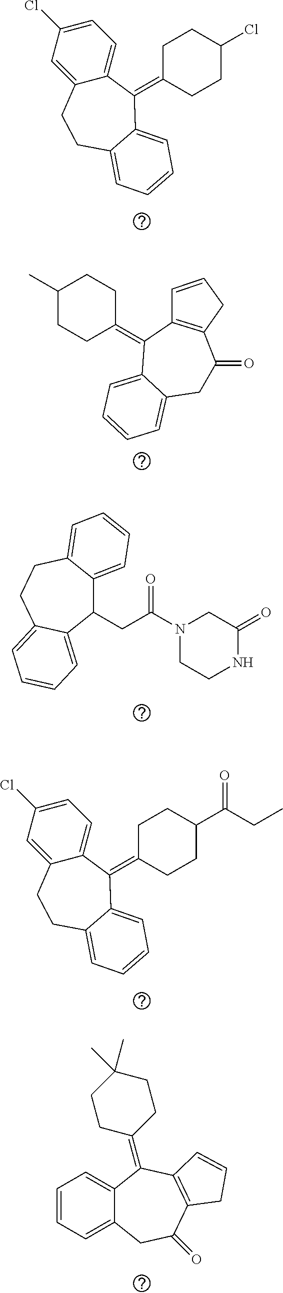 [SMILES: Clc1ccc2c(c1)CCc1ccccc1C2=C1CCC(Cl)CC1 ; CC1CCC(=C2C3=C(CC=C3)C(=O)Cc3ccccc32)CC1 ; O=C1CN(C(=O)CC2c3ccccc3CCc3ccccc32)CCN1 ; CCC(=O)C1CCC(=C2c3ccccc3CCc3cc(Cl)ccc32)CC1 ; CC1(C)CCC(=C2C3=C(CC=C3)C(=O)Cc3ccccc32)CC1]

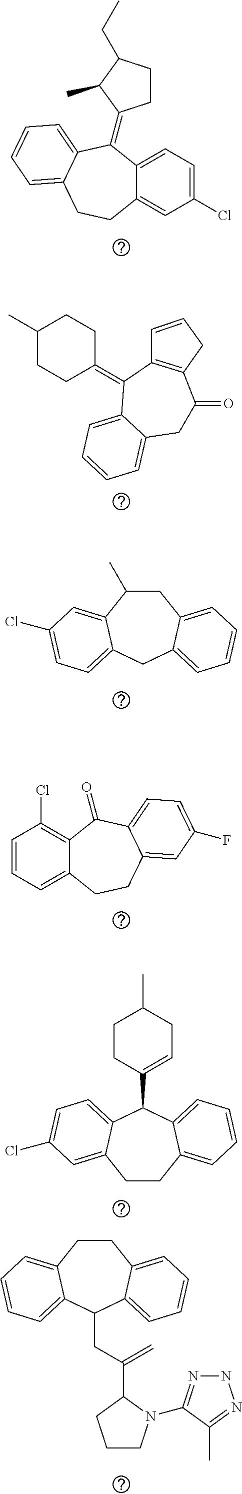 [SMILES: CCC1CC/C(=C2/c3ccccc3CCc3cc(Cl)ccc32)[C@H]1C ; CC1CCC(=C2C3=C(CC=C3)C(=O)Cc3ccccc32)CC1 ; CC1Cc2ccccc2Cc2ccc(Cl)cc21 ; O=C1c2ccc(F)cc2CCc2cccc(Cl)c21 ; CC1CC=C([C@H]2c3ccccc3CCc3cc(Cl)ccc32)CC1 ; C=C(CC1c2ccccc2CCc2ccccc21)C1CCCN1c1n[nH]nc1C]

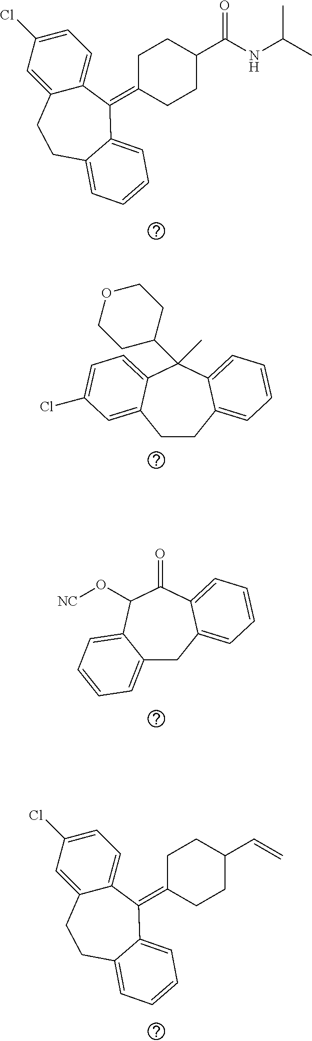 [SMILES: CC(C)NC(=O)C1CCC(=C2c3ccccc3CCc3cc(Cl)ccc32)CC1 ; CC1(C2CCOCC2)c2ccccc2CCc2cc(Cl)ccc21 ; N#COC1C(=O)c2ccccc2Cc2ccccc21 ; C=CC1CCC(=C2c3ccccc3CCc3cc(Cl)ccc32)CC1]

### Methods of Treatment

The IWS1 inhibitors described herein can be prepared using synthetic methodologies known in the art. It will be appreciated by one skilled in the art that the processes described are not the exclusive means by which compounds provided herein may be synthesized and that a broad repertoire of synthetic organic reactions is available to be potentially employed in synthesizing compounds provided herein. The person skilled in the art knows how to select and implement appropriate synthetic routes. Suitable synthetic methods of starting materials, intermediates and products may be identified by reference to the literature, including reference sources such as: Advances in Heterocyclic Chemistry, Vols. 1-107 (Elsevier, 1963-2012); Journal of Heterocyclic Chemistry Vols. 1-49 (Journal of Heterocyclic Chemistry, 1964-2012); Carreira, et al. (Ed.) Science of Synthesis, Vols. 1-48 (2001-2010) and Knowledge Updates KU2010/1-4; 2011/1-4; 2012/1-2 (Thieme, 2001-2012); Katritzky, et al. (Ed.) Comprehensive Organic Functional Group Transformations, (Pergamon Press, 1996); Katritzky et al. (Ed. ); Comprehensive Organic Functional Group Transformations II (Elsevier, 2^{nd} Edition, 2004); Katritzky et al. (Ed.), Comprehensive Heterocyclic Chemistry (Pergamon Press, 1984); Katritzky et al., Comprehensive Heterocyclic Chemistry II, (Pergamon Press, 1996); Smith et al., March's Advanced Organic Chemistry: Reactions, Mechanisms, and Structure, 6^{th} Ed. (Wiley, 2007); Trost et al. (Ed.), Comprehensive Organic Synthesis (Pergamon Press, 1991).

The reactions for preparing IWS1 inhibitors described herein can be carried out in suitable solvents which can be readily selected by one of skill in the art of organic synthesis. Suitable solvents can be substantially non-reactive with the starting materials (reactants), the intermediates, or products at the temperatures at which the reactions are carried out, (e.g., temperatures which can range from the solvent's freezing temperature to the solvent's boiling temperature). A given reaction can be carried out in one solvent or a mixture of more than one solvent. Depending on the particular reaction step, suitable solvents for a particular reaction step can be selected by the skilled artisan.

Preparation of IWS1 inhibitors described herein can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3^{rd} Ed., Wiley & Sons, Inc., New York (1999).

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatographic methods such as high performance liquid chromatography (HPLC), liquid chromatography-mass spectroscopy (LCMS), or thin layer chromatography (TLC). Compounds can be purified by those skilled in the art by a variety of methods, including high performance liquid chromatography (HPLC) and normal phase silica chromatography.

As described above, IWS1 inhibitors, including those described herein, can be administered to treat or prevent cancer. As used herein, the term “cancer” refers to a class of diseases of mammals characterized by uncontrolled cellular growth. The term “cancer” is used interchangeably with the terms “malignancy,” “hyperproliferation” and “neoplasm.” Cancer includes all types of hyperproliferative growth, hyperplasic growth, neoplastic growth, cancerous growth or oncogenic processes, metastatic tissues or malignantly transformed cells, tissues, or organs, irrespective of histopathologic type or stage of invasiveness. Illustrative examples include, lung, prostate, head and neck, breast and colorectal cancer, melanomas and gliomas (such as a high grade glioma, including glioblastoma multiforme (GBM), the most common and deadliest of malignant primary brain tumors in adult humans).

Examples of proliferative disorders (cancers) that can be treated using the methods disclosed herein include: lung cancer (including small cell lung cancer and non small cell lung cancer), other cancers of the pulmonary system, medulloblastoma and other brain cancers, pancreatic cancer, basal cell carcinoma, breast cancer, prostate cancer and other genitourinary cancers, gastrointestinal stromal tumor (GIST) and other cancers of the gastrointestinal tract, colon cancer, colorectal cancer, ovarian cancer, cancers of the hematopoietic system (including multiple myeloma, acute lymphocytic leukemia, acute myelocytic leukemia, chronic myelocytic leukemia, chronic lymphocytic leukemia, Hodgkin lymphoma, and non-Hodgkin lymphoma, and myelodysplastic syndrome), polycythemia Vera, Waldenstrom's macroglobulinemia, heavy chain disease, soft-tissue sarcomas, such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, melanoma, and other skin cancers, adenocarcinoma, sweat gland carcinoma, sebaceous gland carcinoma, papillary carcinoma, papillary adenocarcinomas, stadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular cancer, bladder carcinoma, and other genitourinary cances, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, endometrial cancer, follicular lymphoma, diffuse large B-cell lymphoma, mantle cell lymphoma, hepatocellular carcinoma, thyroid cancer, gastric cancer, esophageal cancer, head and neck cancer, small cell cancers, essential thrombocythemia, agnogenic myeloid metaplasia, hypereosinophilic syndrome, systemic mastocytosis, familiar hypereosinophilia, chronic eosinophilic leukemia, thyroid cancer, neuroendocrine cancers, and carcinoid tumors.

In some embodiments, the cancer can comprise a solid tumor. As used herein, the phrase “solid tumor” includes, for example, lung cancer, head and neck cancer, brain cancer, oral cancer, colorectal cancer, breast cancer, prostate cancer, pancreatic cancer, and liver cancer. Other types of solid tumors are named for the particular cells that form them, for example, sarcomas formed from connective tissue cells (for example, bone cartilage, fat), carcinomas formed from epithelial tissue cells (for example, breast, colon, pancreas) and lymphomas formed from lymphatic tissue cells (for example, lymph nodes, spleen, thymus).

Metastases of the aforementioned cancers can also be treated in accordance with the methods described herein. Thus, in some embodiments, the cancer is a metastatic cancer. “Metastatic cancer,” as used herein, refers to a cancer that has spread from its primary site of origin or where it first formed to other areas of a subject's body. Examples of metastatic cancers include metastatic breast cancer, metastatic colon cancer, metastatic lung cancer, metastatic pancreatic cancer, metastatic prostate cancer, metastatic bone cancer, metastatic blood cancer, metastatic brain cancer, and metastatic liver cancer.

In certain embodiments, the cancer can comprise a soft tissue sarcoma.

In certain embodiments, the cancer can comprise a liposarcoma (LPS). Examples of liposarcomas include, for example, atypical lipomatous tumors (ALTs), well-differentiated liposarcomas (WDLs), myxoid liposarcomas (MLS) including round cell liposarcomas, pleomorphic liposarcomas (PLS), myxoid pleomorphic liposarcomas (originally termed pleomorphic myxoid liposarcomas), and dedifferentiated LPS (ddLPS). In certain embodiments, the cancer can comprise dedifferentiated LPS (ddLPS)

As described herein, IWS1, a transcription elongation and alternative splicing factor, can be elevated in certain cancers, including dedifferentiated LPS (ddLPS) tumor samples. Further, elevated levels of IWS1 can be associated with worse survival and a shorter time to relapse in certain cancers, including ddLPS.

In some embodiments, the cancer can be characterized by elevated levels of IWS1.

In certain embodiments, the cancer can comprise a soft tissue sarcoma characterized by elevated levels of IWS1.

In certain embodiments, the cancer can comprise a liposarcoma characterized by elevated levels of IWS1. In certain embodiments, the cancer can comprise dedifferentiated LPS characterized by elevated levels of IWS1.

In some embodiments, the IWS1 inhibitor reduces the migration ability of cancer cells in the subject, reduces the invasiveness of cancer cells in the subject, reduces metastasis of the cancer, or a combination thereof.

Also provided herein are methods for preventing metastisis of a liposarcoma in a subject or reducing the rate of metastisis of a liposarcoma. These methods can comprise administering to the subject an effective amount of an IWS1 inhibitor to prevent metastisis of the liposarcoma or reduce the rate of metastisis of the liposarcoma. In certain embodiments, the liposarcoma can comprise dedifferentiated LPS (ddLPS).

Also provided herein are methods for reducing the migration of liposarcoma cells in a subject. These methods can comprise administering to the subject an effective amount of an IWS1 inhibitor to reduce the migration of the liposarcoma cells. In certain embodiments, the liposarcoma can comprise dedifferentiated LPS (ddLPS).

Also provided herein are methods for reducing the invasiveness of liposarcoma cells in a subject. These methods can comprise administering to the subject an effective amount of an IWS1 inhibitor to reduce the invasiveness of the liposarcoma cells. In certain embodiments, the liposarcoma can comprise dedifferentiated LPS (ddLPS).

In the methods described herein, the subject receiving this treatment can be any animal in need, including primates, in particular humans, and other mammals such as equines, cattle, swine and sheep; and poultry and pets in general. In certain embodiments, the subject can be a human.

IWS1 inhibitors, including the ISW1 inhibitors disclosed herein, can be combined with other cancer treatments. For example, they can be combined with surgical treatments; radiation; biotherapeutics (such as interferons, cytokines—e.g., Interferon a, Interferon y, and tumor necrosis factor, hematopoietic growth factors, monoclonal serotherapy, vaccines and immunostimulants); antibodies (e.g., Avastin, Erbitux, Rituxan, and Bexxar); endocrine therapy (including peptide hormones, corticosteroids, estrogens, androgens and aromatase inhibitors); anti-estrogens (e.g., Tamoxifen, Raloxifene, and Megestrol); LHRH agonists (e.g., goscrclin and Leuprolide acetate); anti-androgens (e.g., flutamide and Bicalutamide); gene therapy; bone marrow transplantation; photodynamic therapies (e.g., vertoporfin (BPD-MA), Phthalocyanine, photosensitizer Pc4, and Demethoxy-hypocrellin A (2BA-2-DMHA)); and chemotherapeutics.

Examples of chemotherapeutics include gemcitabine, methotrexate, taxol, mercaptopurine, thioguanine, hydroxyurea, cytarabine, cyclophosphamide, ifosfamide, nitrosoureas, cisplatin, carboplatin, mitomycin, dacarbazine, procarbizine, etoposides, prednisolone, dexamethasone, cytarbine, campathecins, bleomycin, doxorubicin, idarubicin, daunorubicin, dactinomycin, plicamycin, mitoxantrone, asparaginase, vinblastine, vincristine, and vinorelbine. Additional agents include nitrogen mustards (e.g., cyclophosphamide, Ifosfamide, Trofosfamide, Chlorambucil, Estramustine, and Melphalan), nitrosoureas (e.g., carmustine (BCNU) and Lomustine (CCNU)), alkylsulphonates (e.g., busulfan and Treosulfan), triazenes (e.g., Dacarbazine and Temozolomide), platinum containing compounds (e.g., Cisplatin, Carboplatin, and oxaliplatin), vinca alkaloids (e.g., vincristine, Vinblastine, Vindesine, and Vinorelbine), taxoids (e.g., paclitaxel and Docetaxol), epipodophyllins (e.g., etoposide, Teniposide, Topotecan, 9-Aminocamptothecin, Camptoirinotecan, Crisnatol, Mytomycin C, and Mytomycin C), anti-metabolites, DHFR inhibitors (e.g., methotrexate and Trimetrexate), IMP dehydrogenase Inhibitors (e.g., mycophenolic acid, Tiazofurin, Ribavirin, and EICAR), ribonucleotide reductase Inhibitors (e.g., hydroxyurea and Deferoxamine), uracil analogs (e.g., Fluorouracil, Floxuridine, Doxifluridine, Ratitrexed, and Capecitabine), cytosine analogs (e.g., cytarabine (ara C), Cytosine arabinoside, and Fludarabine), purine analogs (e.g., mercaptopurine and Thioguanine), Vitamin D3 analogs (e.g., EB 1089, CB 1093, and KH 1060), isoprenylation inhibitors (e.g., Lovastatin), dopaminergic neurotoxins (e.g., 1-methyl-4-phenylpyridinium ion), cell cycle inhibitors (e.g., staurosporine), actinomycins (e.g., Actinomycin D and Dactinomycin), bleomycins (e.g., bleomycin A2, Bleomycin B2, and Peplomycin), anthracyclines (e.g., daunorubicin, Doxorubicin (adriamycin), Idarubicin, Epirubicin, Pirarubicin, Zorubicin, and Mitoxantrone), MDR inhibitors (e.g., verapamil), Ca²⁺ ATPase inhibitors (e.g., thapsigargin), imatinib, thalidomide, lenalidomide, erlotinib, gefitinib, sorafenib, and sunitinib, and proteasome inhibitors, including bortezomib.

When IWS1 inhibitors, including the ISW1 inhibitors disclosed herein, are administered in combination with other treatments, such as additional therapeutics or with radiation or surgery, the doses of each agent or therapy will in most instances be lower than the corresponding dose for single-agent therapy. Also, in general, IWS1 inhibitors, including the ISW1 inhibitors disclosed herein, and the second therapeutic agent do not have to be administered in the same pharmaceutical composition, and may, because of different physical and chemical characteristics, be administered by different routes. For example, one compound can be administered orally, while the second therapeutic is administered intravenously. The determination of the mode of administration and the advisability of administration, where possible, in the same pharmaceutical composition, is well within the knowledge of the skilled clinician. The initial administration can be made according to established protocols known in the art, and then, based upon the observed effects, the dosage, modes of administration and times of administration can be modified by the skilled clinician.

IWS1 inhibitors, including the ISW1 inhibitors disclosed herein, and the second therapeutic agent and/or radiation may be administered concurrently (e.g., simultaneously, essentially simultaneously or within the same treatment protocol) or sequentially (i.e., one followed by the other, with an optional time interval in between), depending upon the nature of the proliferative disease, the condition of the patient, and the actual choice of second therapeutic agent and/or radiation to be administered.

If IWS1 inhibitors, including the ISW1 inhibitors disclosed herein, and the second therapeutic agent and/or radiation are not administered simultaneously or essentially simultaneously, then the optimum order of administration may be different for different conditions. Thus, in certain situations the IWS1 inhibitors, including the ISW1 inhibitors disclosed herein, may be administered first followed by the administration of the second therapeutic agent and/or radiation; and in other situations the second therapeutic agent and/or radiation may be administered first followed by the administration of an IWS1 inhibitor, including an ISW1 inhibitor disclosed herein. This alternate administration may be repeated during a single treatment protocol. The determination of the order of administration, and the number of repetitions of administration of each therapeutic agent during a treatment protocol, is well within the knowledge of the skilled physician after evaluation of the disease being treated and the condition of the patient. For example, the second therapeutic agent and/or radiation may be administered first, especially if it is a cytotoxic agent, and then the treatment continued with the administration of an IWS1 inhibitor, including an ISW1 inhibitor disclosed herein, followed, where determined advantageous, by the administration of the second therapeutic agent and/or radiation, and so on until the treatment protocol is complete.

As used herein, the phrase “effective amount” refers to the amount of active compound or pharmaceutical agent that elicits the biological or medicinal response that is being sought in a tissue, system, animal, individual or human by a researcher, veterinarian, medical doctor or other clinician. An effective amount of a compound provided herein can range, for example, from about 0.01 mg/kg to about 1000 mg/kg, (e.g., from about 0.1 mg/kg to about 100 mg/kg, from about 1 mg/kg to about 100 mg/kg). Effective doses will also vary depending on route of administration, as well as the possibility of co-usage with other agents.

### Combination Therapies

As discussed above, the methods provided herein further comprise administering one or more additional therapeutic agents to the subject. In some embodiments, each of the one or more additional therapeutic agents is independently selected from the group consisting of a steroid, an anti-allergic agent, an anesthetic (e.g., for use in combination with a surgical procedure), an immunosuppressant, an anti-microbial agent, an anti-inflammatory agent, and a chemotherapeutic agent.

Example steroids include, but are not limited to, corticosteroids such as cortisone, dexamethasone, hydrocortisone, methylprednisolone, prednisolone, and prednisone.

Example anesthetics include, but are not limited to local anesthetics such as lidocaine, procain, and ropivacaine.

Example immunosuppressants include, but are not limited to, azathioprine, chlorambucil, cyclophosphamide, cyclosporine, daclizumab, infliximab, methotrexate, and tacrolimus.

Example anti-microbial agents include, but are not limited to, aminoglycosides (e.g., gentamicin, neomycin, and streptomycin), penicillins (e.g., amoxicillin and ampicillin), and macrolides (e.g., erythromycin).

Example anti-inflammatory agents include, but are not limited to, aspirin, choline salicylates, celecoxib, diclofenac potassium, diclofenac sodium, diclofenac sodium with misoprostol, diflunisal, etodolac, fenoprofen, flurbiprofen, ibuprofen, ketoprofen, meclofenamate sodium, mefenamic acid, nabumetone, naproxen, naproxen sodium, oxaprozin, piroxican, rofecoxib, salsalate, sodium salicylate, sulindac, tolmetin sodium, and valdecoxib.

Example chemotherapeutics include, but are not limited to, proteosome inhibitors (e.g., bortezomib), thalidomide, revlimid, and DNA-damaging agents such as melphalan, doxorubicin, cyclophosphamide, vincristine, etoposide, carmustine, and the like. For example, one or more of the following agents may be used in combination with the compounds provided herein and are presented as a non-limiting list: a cytostatic agent, cisplatin, taxol, etoposide, irinotecan, topotecan, paclitaxel, docetaxel, epothilones, tamoxifen, 5-fluorouracil, temozolomide, cyclophosphamide, gefitinib, erlotinib hydrochloride, imatinib mesylate, gemcitabine, uracil mustard, chlormethine, ifosfamide, chlorambucil, pipobroman, triethylenemelamine, triethylenethiophosphoramine, busulfan, lomustine, streptozocin, dacarbazine, floxuridine, cytarabine, 6-mercaptopurine, 6-thioguanine, fludarabine phosphate, oxaliplatin, folinic acid, pentostatin, vinblastine, vindesine, bleomycin, dactinomycin, daunorubicin, doxorubicin, epirubicin, idarubicin, mithramycin, deoxycoformycin, mitomycin-C, L-asparaginase, teniposide, 17α-ethinylestradiol, diethylstilbestrol, testosterone, prednisone, fluoxymesterone, dromostanolone propionate, testolactone, megestrol acetate, methyltestosterone, triamcinolone, chlorotrianisene, hydroxyprogesterone, aminoglutethimide, estramustine, medroxyprogesteroneacetate, leuprolide, flutamide, toremifene, goserelin, carboplatin, hydroxyurea, amsacrine, procarbazine, mitotane, mitoxantrone, levamisole, vinorelbine, anastrazole, letrozole, capecitabine, reloxafine, hexamethylmelamine, bevacizumab, bexxar, velcade, zevalin, trisenox, xeloda, porfimer, erbitux, thiotepa, altretamine, trastuzumab, fulvestrant, exemestane, ifosfamide, rituximab, alemtuzumab, clofarabine, cladribine, aphidicolin, sunitinib, dasatinib, tezacitabine, triapine, trimidox, amidox, bendamustine, and ofatumumab.

### Pharmaceutical Compositions

When employed as pharmaceuticals, IWS1 inhibitors, including the ISW1 inhibitors disclosed herein, can be administered in the form of pharmaceutical compositions. These compositions can be prepared as described herein or elsewhere, and can be administered by a variety of routes, depending upon whether local or systemic treatment is desired and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral, or parenteral. Parenteral administration includes intravenous, intraarterial, subcutaneous, intraperitoneal intramuscular or injection or infusion; or intracranial, (e.g., intrathecal or intraventricular, administration). Parenteral administration can be in the form of a single bolus dose, or may be, for example, by a continuous perfusion pump. In some embodiments, the compounds provided herein, or a pharmaceutically acceptable salt thereof, are suitable for parenteral administration. In some embodiments, the compounds provided herein are suitable for intravenous administration. In some embodiments, the compounds provided herein are suitable for oral administration. In some embodiments, the compounds provided herein are suitable for topical administration.

Pharmaceutical compositions and formulations for topical administration may include, but are not limited to, transdermal patches, ointments, lotions, creams, gels, drops, suppositories, sprays, liquids and powders. Conventional pharmaceutical carriers, aqueous, powder or oily bases, thickeners and the like may be necessary or desirable. In some embodiments, the pharmaceutical compositions provided herein are suitable for parenteral administration. In some embodiments, the pharmaceutical compositions provided herein are suitable for intravenous administration. In some embodiments, the pharmaceutical compositions provided herein are suitable for oral administration. In some embodiments, the pharmaceutical compositions provided herein are suitable for topical administration.

Also provided are pharmaceutical compositions which contain, as the active ingredient, a compound provided herein in combination with one or more pharmaceutically acceptable carriers (e.g. excipients). In making the pharmaceutical compositions provided herein, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be, for example, in the form of tablets, pills, powders, lozenges, sachets, cachets, elixirs, suspensions, emulsions, solutions, syrups, aerosols (as a solid or in a liquid medium), ointments, soft and hard gelatin capsules, suppositories, sterile injectable solutions, and sterile packaged powders.

Some examples of suitable excipients include, without limitation, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include, without limitation, lubricating agents such as talc, magnesium stearate, and mineral oil; wetting agents; emulsifying and suspending agents; preserving agents such as methyl- and propylhydroxy-benzoates; sweetening agents; flavoring agents, or combinations thereof.

The active compound can be effective over a wide dosage range and is generally administered in an effective amount. It will be understood, however, that the amount of the compound actually administered will usually be determined by a physician, according to the relevant circumstances, including the condition to be treated, the chosen route of administration, the actual compound administered, the age, weight, and response of the individual subject, the severity of the subject's symptoms, and the like.

The compositions provided herein can be administered one from one or more times per day to one or more times per week; including once every other day. The skilled artisan will appreciate that certain factors can influence the dosage and timing required to effectively treat a subject, including, but not limited to, the severity of the disease or disorder, previous treatments, the general health and/or age of the subject, and other diseases present. Moreover, treatment of a subject with a therapeutically effective amount of a compound described herein can include a single treatment or a series of treatments.

Dosage, toxicity and therapeutic efficacy of the compounds provided herein can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds exhibiting high therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

### EXAMPLES

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results.

### Example 1: Phosphorylation of IWS1 by AKT Maintains Liposarcoma Tumor Heterogeneity through Preservation of Cancer Stem Cell Phenotypes and Mesenchymal-Epithelial Plasticity

Chemotherapy remains the mainstay of treatment for patients with advanced liposarcoma (LPS), but response rates are only 25% and the overall survival at 5 years is dismal at 20-34%. Translation of other therapies have not been successful and there has been no significant improvement in prognosis for nearly 20 years. The aberrant activation of the phosphatidylinositol 3-kinase (PI3K)/AKT pathway has been implicated in the aggressive clinical behavior LPS and in resistance to chemotherapy, but the precise mechanism remains elusive and efforts to target AKT clinically have failed. In this example, we show that the AKT-mediated phosphorylation of the transcription elongation factor IWS1, promotes the maintenance of cancer stem cells in both cell and xenograft models of LPS. In addition, phosphorylation of IWS1 by AKT contributes to a “metastable” cell phenotype, characterized by mesenchymal/epithelial plasticity. The expression of phosphorylated IWS1 also promotes anchorage-dependent and independent growth, cell migration, invasion, and tumor metastasis. In patients with LPS, IWS1 expression is associated with reduced overall survival, increased frequency of recurrence, and shorter time to relapse after resection. These findings indicate that IWS1-mediated transcription elongation is an important regulator of human LPS pathobiology in an AKT-dependent manner and implicate IWS1 as an important molecular target to treat LPS.

### INTRODUCTION

While rare, retroperitoneal liposarcomas (LPS) are locally aggressive tumors that present significant clinical challenges and are associated with a dismal prognosis (1, 2). Surgical resection remains the only chance for long-term survival (3, 4). Yet, tumor recurrence occurs in most patients (4, 5). Doxorubicin-based chemotherapy remains the mainstay of treatment for patients with unresectable LPS, but response rates are only 25%, and the 5-year survival is only 20-30% (6, 7). Mutations driving LPS are known, but therapies directed toward specific targets have yielded results far below expectations (5, 8). The development of novel therapies has been limited, and there has been no significant improvement in prognosis for nearly 20 years. Therefore, the identification of novel drivers of tumorigenesis may help identify novel targets for therapy remains an important unmet need (1, 8).

A major challenge hampering the development of effective LPS treatments is tumor heterogeneity and the remarkable plasticity of LPS cancer cells (9-11). While LPS are mesenchymal tumors, recent evidence suggests that sarcoma cells may exhibit a “metastable” phenotype, characterized by the transition of tumor cells between mesenchymal and epithelial-like states (12, 13). This plasticity appears to be a feature of tumors with more aggressive biology, as their cells can undergo mesenchymal to epithelial transition (MET), giving rise to highly proliferative cells in the primary tumor, or epithelial to mesenchymal transition (EMT), giving rise to migratory, invasive, and metastatic cells. Recent evidence also suggests a strong link between EMT/MET and the maintenance of cancer stem cells (CSC) (12, 14-16). The EMT/MET signaling programs confer CSC-like characteristics to cells within the tumor, thereby increasing tumor heterogeneity, which has been linked to therapeutic resistance in many different types of cancer including LPS (17-19). A more comprehensive understanding of the drivers of these programs in LPS may help develop strategies to reduce the resistance of LPS to currently available therapies.

Aberrant activation of the PI3K/AKT pathway has long been implicated in the aggressive clinical behavior of LPS and was recently shown to contribute to the self-renewal of LPS CSCs (20-23). Specifically, pharmacologic inhibition and genetic knockdown of AKT1 and AKT2 in the human dedifferentiated LPS cell line, DDLS8817, decreased Nanog levels and spheroid formation in suspension cultures and reversed resistance to doxorubicin treatment and radiation therapy (22). These findings are consistent with a previous report showing that inhibition of the PI3K/AKT pathway reduces expression of EMT markers and the abundance of CSCs in prostate cancer cell lines, rendering them more sensitive to irradiation (24). PI3K/AKT1 signaling is also required for transforming growth factor beta-induced EMT in breast cancer, and for the maintenance of EMT in pancreatic cancer (25, 26). While these findings highlight the pivotal role of AKT in driving oncogenesis and chemoresistance by promoting EMT and the maintenance of CSCs, efforts to target the PI3K/AKT pathway have been met with limited success (NCT02987959, NCT01048723). To date, no PI3K/AKT inhibitor has been tested in a phase III clinical trial.

Recently, AKT has been shown to promote tumor growth through activating the transcription elongation factor IWS1 (27). IWS1 functions to recruit cofactors to the RNA polymerase II (RNAPII) complex to regulate mRNA alternative splicing (28-30). The processing of mRNA precursors is highly regulated and occurs before the translation of almost all eukaryotic genes (31-33). By modulating RNA splicing, IWS1 could generate oncoproteins that promote LPS (34-37). Phosphorylation of IWS1 at Ser 720/Thr 721 by AKT, recruits the Histone H3K36 trimethyl transferase SETD2 to the SPT6/IWS1 CTD complex. Following its recruitment, SETD2 trimethylates histone H3 at K36 in the body of target genes during transcriptional elongation, catalyzing multiple steps in RNA processing (27, 28, 38). These activities of IWS1 promote the aggressiveness of lung cancer cells (27). Other studies have shown that IWS1 is a regulator of embryonic development and is required for the early stages of embryogenesis in mice (39), where it is required for Nanog expression (28). Nanog is a transcription factor that plays a critical role in maintenance of CSCs in LPS and has been shown to confer LPS resistance to both chemotherapy and radiation therapy (22, 28). Understanding how the phosphorylation of IWS1 by AKT contributes to LPS tumor biology, could identify novel AKT-dependent targets whose inhibition may materialize the unfulfilled promise of AKT inhibition.

### Materials and Methods

Clinical specimens. A total of 33 primary or recurrent LPS tumor tissue samples and 20 matched adjacent adipose tissue samples, were acquired from patients who underwent surgical resections from 1992 to 2019 and included 18 of well-differentiated retroperitoneal LPS and 15 of dedifferentiated retroperitoneal LPS. Each case was validated and classified by an experienced bone and soft tissue pathologist (OHI) based on the most recent World Health Organization (WHO) classification of tumors. All specimens were frozen and stored at −80 °C immediately.

Immunohistochemistry. Immunohistochemistry was carried out on 4 m pathological paraffin sections and stained with a DAB Peroxidase (HRP) Substrate Kit (Vector Labs, Cat No. SK-4100) for the detection of p-AKT (T308) (1:100 dilution; Cell Signaling Technology, Cat No. 13038), p-AKT (S473) (1:100 dilution; Cell Signaling Technology, Cat No. 4060), IWS1 (1:100 dilution; Cell Signaling Technology, Cat No. 5681), Vimentin (1:100 dilution; Cell Signaling Technology, Cat No. 5741S), pan-Cytokeratin (1:100 dilution; Abcam, Cat No. ab80826) and Ki-67 (1:100 dilution; Cell Signaling Technology, Cat No. 90275) according to the manufacturer's protocols. The staining intensity was graded independently by 2 experienced pathologists.

Cell culture. The human LPS cell line SW872 was obtained from ATCC, and the Lipo863, Lipo224, Lipo815 cell lines were established in our laboratory as previously reported (53). Frozen stocks are re-analyzed every 6 months for any changes in cell biology. All cell lines utilized in the study have been tested for mycoplasma and undergo testing every month utilizing both biochemical chemiluminescent assay and PCR based assay systems. All cells were cultured in Dulbecco's modified Eagle's medium (DMEM; Corning, Cat No. 10-013-CV) supplemented with 10% fetal bovine serum (FBS; Corning, Cat No. 35-011-CV) and 1% penicillin/streptomycin (Gibco, Cat No. 15140-122) at 37 °C in a humidified atmosphere containing 5% CO2. Cell lines were also periodically checked for mycoplasma, using the PCR mycoplasma detection kit (ABM, Cat No. G238). All experiments were carried out in mycoplasma-free cultures.

Transfections and infections. Packaging plasmid psPAX2 (Addgene #12260), enveloping plasmid pMΔ2.G (Addgene #12259), and three lentiviral vectors including shRNA against human IWS1, pLx304 Flag-IWS1, pLx304 Flag-IWS1 S720A/T721A mutant (shRNA resistant clones) were used (27). Lentiviruses were packaged in HEK-293T cells by transient transfection of lentiviral constructs and packaging constructs using 2×HEPES Buffered Saline (Sigma Cat. No 51558) and calcium-phosphate precipitation method. Lentiviral supernatants were collected at 48 h post transfection and filtered through a 0.45 μm filter. LPS cells were infected with viral particles in the presence of 8 μg/ml polybrene (Sigma, Cat No. 107689) for 8 h and selected for stable infection with 2 μg/ml puromycin (MilliporeSigma, Cat No 54-041-1.) or 5 μg/ml blasticidin (MilliporeSigma, Cat No. 20-335-1). Cells infected with multiple constructs were selected for infection with the first construct, prior to the next infection. The pLKO.1 TRC control vector was utilized as a non-hairpin control (Addgene #10879) (27).

Cell Viability Assay. LPS cells were seeded and cultured in 96-well plates at a density of 2×10⁴/well for 24 h. Cell viability was assessed by the Cell counting kit-8 (CCK8) assay kit (Dojindo Molecular Technologies, Cat No. CK0401) with the absorbance measured at 450 nm using a multi-mode microplate reader.

Colony Formation Assay. For the plate colony formation assay, stable transfected SW872 cells (0.5×10³) or Lipo863 cells (1×10³) were seeded in 60-mm culture dishes and cultured in DMEM medium containing 10% FBS. Following incubation at 37 °C for 2 weeks, the colonies were fixed with methanol for 20 min and stained with 0.5% crystal violet for 10 min. For Soft Agar colony formation assay, stable infected SW872 cells or Lipo863 cells were mixed into DMEM medium containing 10% FBS and 0.35% Noble agar (Difco™ Agar Noble, BD Biosciences, Cat. No DF0142-15-2) and seeded in 60-mm culture dishes pre-coated with 0.6% noble agarose in DMEM medium containing 10% FBS. The cells in the agar dish were incubated at 37 °C and then stained with MTT for 20 min. The images of the colonies were obtained by a scanner (Epson America Inc., Long Beach, CA) and the number of colonies was quantified with Image J software.

Wound healing assay. Stable transfected LPS cells were seeded and inoculated in 6-well plates until the cell reached more than 95% confluence. The monolayer was scratched with a sterile 100 L pipette tip and the wound closure was observed under an inverted microscope at 0 and 48 h after wounding.

Transwell Assay. Stable transfected LPS cells (2×10⁴) were placed into the upper chamber (8.0-μm pore size; Corning Inc., Cat No. 3422) uncoated or pre-coated with Matrigel (Corning Inc., Cat. No 354234) to identify the cell migration and invasion. Following incubation for 24 h, the number of cells penetrating to the underside of the membrane were stained with crystal violet and visualized with an inverted microscope.

Quantitative polymerase chain reaction (RT-qPCR). Total RNA was extracted from LPS cells using a NucleoSpin RNA kit (Macherey-Nagel, Duren, Cat. No 740955.5) and synthesized into cDNA using 5 xPrime Script RT Master Mix (Takara Bio, Inc., Cat. No RR036A) under the following conditions: 37 °C for 15 min, 85 °C for 5 sec and 4 °C. RT-qPCR was conducted on the StepOne Real-Time PCR System (Applied Biosystems, Cat. No A25742) with specific primers and SYBR green mix under the following conditions: 15 min at 95 °C, followed by 40 cycles of 5 sec at 95 °C, 30 s at 60 °C and 60 sec at 72 °C. Relative expression was evaluated using the 2^{-ΔΔt} method with GAPDH as an endogenous control. The primers were listed as follows: IWS1 forward, 5′-GAACAGCACTGGTGGTCAGACACC-3′ (SEQ. ID NO:1) and reverse, 5′-TGGCACCAGTGTCCGGATTA-3′ (SEQ. ID NO:2); CDK2 forward, 5′-ATGGATGCCTCTGCTCTCACTG-3′ (SEQ. ID NO:3) and reverse, 5′-CCCGATGAGAATGGCAGAAAGC-3′ (SEQ. ID NO:4); CCNA2 (Cyclin A2) forward, 5′-CTCTACACAGTCACGGGACAAAG-3′ (SEQ. ID NO:5) and reverse, 5′-CTGTGGTGCTTTGAGGTAGGTC-3′ (SEQ. ID NO:6); GAPDH forward, 5′-TTCGACAGTCAGCCGCATCTTCTT-3′ (SEQ. ID NO:7) and reverse, 5′-CAGGCGCCCAATACGACCAAATC-3′ (SEQ. ID NO:8).

Western blot. Total protein of LPS tissue and cells were lysed by T-PER Tissue Protein Extraction Reagent (Thermo Scientific, Cat. No 78510) or CelLytic™ M cell lysis reagent (Sigma-Aldrich, Cat. No C₂₉₇₈) supplemented with Halt™ Protease and Phosphatase Inhibitor Cocktail (Thermo Scientific, Cat. No 78438 and 78428), separately. Equal amounts of proteins were subjected to SDS-PAGE gel and transferred onto nitrocellulose membrane. After blocking with 5% BSA, the membranes were incubated with primary antibodies specific for AKT1 (2938S), AKT2 (3063S), AKT3 (14982S), p-AKT (T308) (13038S), p-AKT (S473) (4060S), pan-AKT (4685S), IWS1 (5681S), E-cadherin (3195S), Occludin (91131S), N-cadherin (13116S), Vimentin (5741S), Slug (9585S), Snail (3879S), KLF4 (12173S), Sox2 (2748S), OCT4 (2750S), Nanog (3580S), and 3-actin (8457S) were purchased from Cell signaling and then incubated with IRDye 800CW-conjugated secondary antibodies (1:5000, LI-COR Biosciences, Cat. No 926-32211). Protein bands were visualized with an Odyssey Infrared Imaging System (LI-COR Biosciences). Antibody specific for p-IWS1 was kindly gifted by Dr. Philip Tsichlis (27).

Flow cytometry analysis of cancer stem cell markers. Stable transfected LPS cells (1×10⁶/ml) were stained with Aldefluor™ kit (Stem Cell Technologies Inc., Cat. No 01700) for 45 min at 37 °C and then incubated with allophycocyanin (APC) conjugated anti-human CD133 antibodies (1:100, Biolegend, Cat. No 372806) for 30 min at 4 °C according to the manufacturer's instructions. A specific ALDH inhibitor Diethylaminobenzaldehyde (DEAB) was used as a negative control (Info). Flow cytometry analysis was conducted on a LSR Fortessa Flow Cytometer (BD Biosciences, San Jose, CA) with FACS Diva Software (Version 8.0.1, BD Pharmingen) and analyzed using Flowjo software (version 10).

In vivo tumorigenesis and metastasis assays. Male athymic nude mice (NCr-nu/nu, 4-6 weeks) were obtained from Target Validation Shared Resource at The Ohio State University and randomization was not used to determine how animals were allocated to experimental groups. For xenograft tumor model, 5×10⁶ shControl, shIWS1, shIWS1/WT-R or shIWS1/MT-R SW872 cells were suspended in 100 μL PBS containing 50% Matrigel and inoculated subcutaneously into the flank of nude mice. Tumor size was measured every 3 days and tumor volume was calculated based on the formula:

$$V = \frac{length \times {width}^{2}}{2} .$$

On day 31, xenografts were harvested, fixed in 10% formalin, and then embedded in paraffin. For the lung metastasis model, 1×10⁶ shControl or shIWS1 SW872 cells were injected into the nude mice through the tail vein. Six weeks after injection, lung tissues with metastatic nodules were observed in gross and confirmed by hematoxylin & eosin (H&E) stanning. No blinding was done during all stages of animal experiment.

Survival analysis on TCGA DDLPS data. The raw read counts (coding and non-coding RNAs) and clinical data for the TCGA-SARC cohort were downloaded via the GDC data portal. Overall, all DDLPS samples (N=58) available from the TCGA-SARC cohort were selected. Raw counts were scaled using the Read Per Million (RPM) mapped reads formula and genes with a geometric mean of RPM<5 were removed because they were not expressed or expressed at very low levels. After the filtering step, we normalized the expressed genes with the Trimmed Mean of M values (TMM) method using the edgeR R package (54) and we selected IWS1 for carrying out OS and DSS analyses by employing a univariate Cox regression model leveraging the survFit function from the Survival R package [https://cran.r-project.org/web/packages/survival/index.html]. The median of IWS1 expression across all TCGA-DDLPS samples was used as threshold to define the group of patients with high and low IWS1 expression. Kaplan-Meier curves were finally generated using the ggsurvplot function from Survminer R package [https://cran.r-project.org/web/packages/survminer/index.html].

Cell cycle analysis. The DNA content of Stable transfected LPS cells was determined using FxCycle™ PI/RNase Staining Solution (Invitrogen; F10797). Cells of 2×10⁵ per sample were fixed and stained with staining solution for 15 min for analyzing DNA content. Then, the cell samples were analyzed using LSR Fortessa Flow Cytometer (BD Biosciences, San Jose, CA) and then analyzed using a FACSDiva software (Version 6.1.3).

Apoptosis analysis. The Stable transfected LPS cells were washed with 1×PBS and resuspended in a binding buffer at a concentration of 2×10⁵ cells. The eBioscience™ Annexin V-FITC Apoptosis Detection Kit (Invitrogen; BMS500FI) was purchased, and the apoptosis detection assay was performed by staining with Annexin V-FITC at room temperature for 10 min and then stained with propidium iodide. The assay was performed with LSR Fortessa Flow Cytometer (BD Biosciences, San Jose, CA), and apoptotic cells were analyzed using a FACSDiva software (Version 6.1.3).

Statistical analysis. Statistical analysis was performed using SPSS 20.0 software (IBM Corp., Armonk, NY, USA) and GraphPad Prism 9.0 (GraphPad Inc., La Jolla, CA, USA). Two-tailed Student's t-test or One-way analysis of variance (ANOVA) was conducted to analyze the significance between variables. Data are presented as mean±standard deviation (SD). Cox proportional hazard model (accounting for age, size of tumor, grade, and differentiation) was used to evaluate the effects of IWS1 expression on overall and disease-free survival. Overall survival was calculated from time of diagnosis to death and recurrent free survival was calculated from time of resection to date of first recurrence. Negative binomial regression was employed to assess the effects of IWS1 on the number of deaths. To minimize the effects of the extreme's values of IWS1, logarithmic transformation was used in the model. P<0.05 was considered as statistically significant difference. All statistical tests we used in every figure are justified as appropriate and the data meet the assumptions of the tests as normal distribution.

### Results

The AKT/IWS1 Axis is Active in Human Liposarcoma and Associated with a Worse Prognosis in Patients with Liposarcoma

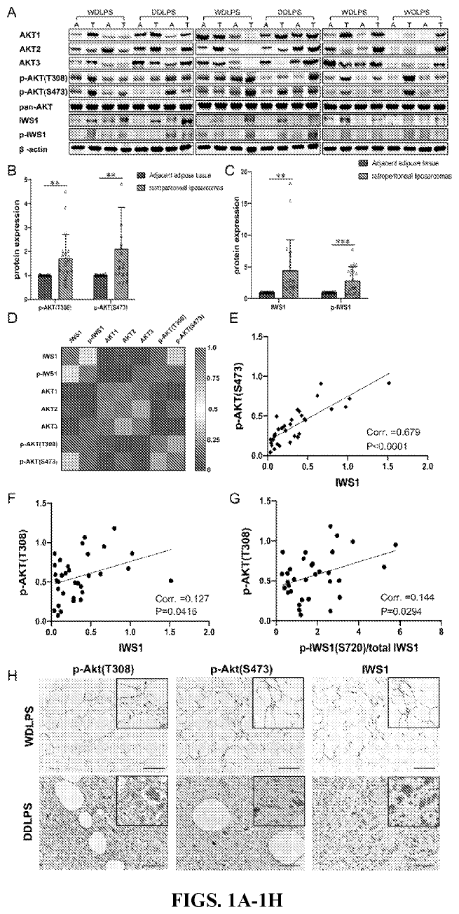

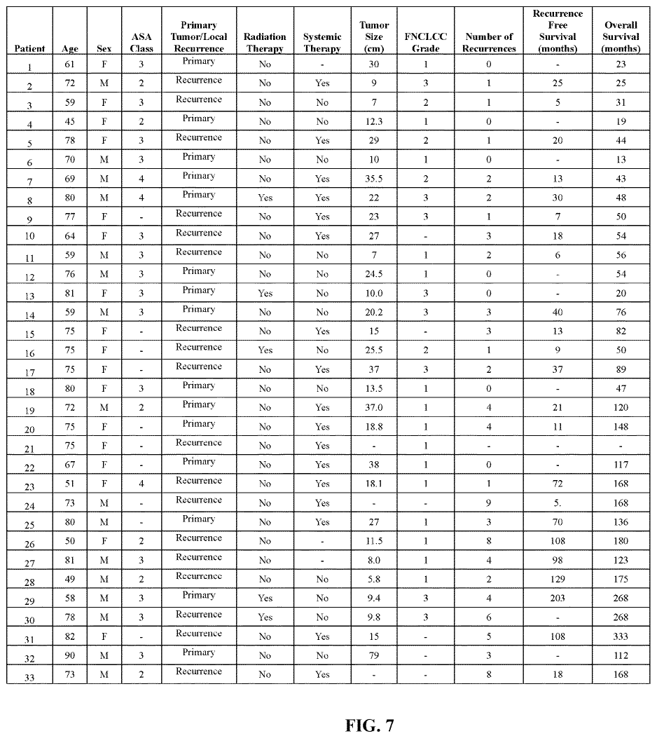

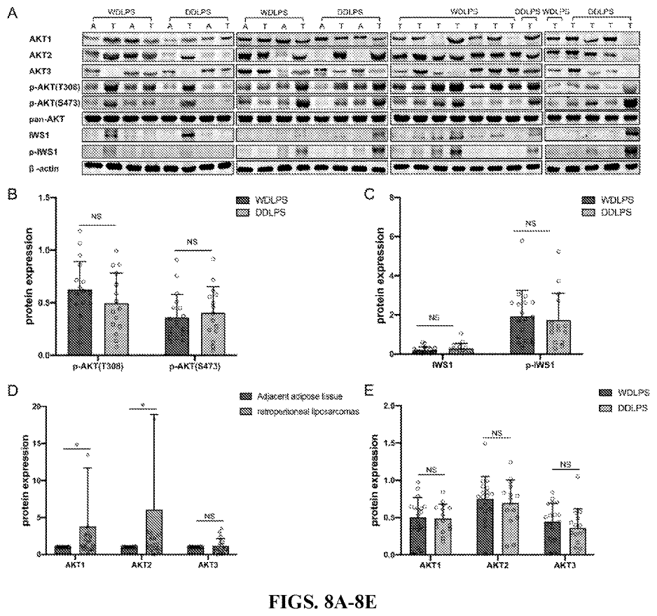

To address the clinical relevance of the AKT/p-IWS1 pathway in patients with LPS, the expression of AKT1, AKT2, AKT3, phospho-AKT (Thr 308), phospho-AKT (Ser 473), IWS1 and phospho-IWS1 (Ser 720) in 33 retroperitoneal LPS tumor specimens and 20 adjacent normal adipose tissue specimens was examined by Western blotting (FIG. 1A and FIG. 8A). The available clinicopathologic characteristics of patients (n=33) are presented in FIG. 7. Increased expression of AKT1, AKT2, AKT3, IWS1 and phospho-IWS1 was observed in most LPS specimens compared to adjacent adipose tissue (FIG. 1B-1C and FIG. 8B-8C). Importantly, phospho-AKT (Thr 308 and Ser 473) was higher in most tumors, even in those in which AKT1, AKT2, and AKT3 were not significantly elevated. By plotting the relative stoichiometry and comparing correlation coefficients of IWS1 and phosphorylated IWS1 with AKT, we found there were significant correlations between IWS1 and phospho-AKT (Thr 308 or Ser 473), as well as phospho-IWS1 and phospho-AKT (Thr 308) (p<0.0001, p=0.0416 and p=0.0294 respectively, by Spearman's rank order correlation) (FIG. 1D-1G). However, there was no difference in the expression of AKT3 when comparing LPS and adjacent adipose tissue (FIG. 8D). There were no differences in the protein expression of AKT1, AKT2, AKT3, IWS1, phospho-IWS1, phospho-AKT (Thr 308) and phospho-AKT when LPS tumors were stratified by state of differentiation (well-differentiated (WD) versus dedifferentiated (DD), FIGS. 8B, 8C, 8E). Since LPS can contain both WD and DD components, the expression of IWS1 and AKT in LPS was further explored by immunohistochemistry. While the expression of IWS1 and phospho-AKT (Thr 308 and Ser 473) were not different in DDLPS and WDLPS when examined by Western blotting, significant differences in the expression of these molecules was detected when specific DDLPS and WDLPS portions of resected tumors were examined by immunohistochemistry (x²=9.44, p=0.002; x²=9.44, p=0.002 and x²=12.86, p<0.001 respectively, FIG. 1H).

TABLE 1
Association of IWS1 expression with disease-free survival
in patients with retroperitoneal liposarcoma.

| | Hazard | Standard | | P | | |
|---|---|---|---|---|---|---|
| | ratio | error | z | value | [95% conf. interval] | |
| logiws1 actin | 1.372 | 0.246 | 1.770 | 0.078 | 0.966 | 1.949 |
| logpiws1 | 1.338 | 0.194 | 2.010 | 0.045 | 1.007 | 1.777 |
| actin | | | | | | |
| logpiws1 | 1.170 | 0.242 | 0.760 | 0.448 | 0.780 | 1.756 |
| IWS1 | | | | | | |

TABLE 2
Association of IWS1 expression with overall survival
in patients with retroperitoneal liposarcoma.

| | Hazard | Standard | | P | | |
|---|---|---|---|---|---|---|
| | ratio | error | z | value | [95% conf. interval] | |
| logiws1 actin | 1.220 | 0.666 | 0.360 | 0.716 | 0.419 | 3.557 |
| logpiws1 | 1.439 | 0.832 | 0.630 | 0.529 | 0.463 | 4.470 |
| actin | | | | | | |
| logpiws1 | 2.925 | 2.822 | 1.110 | 0.266 | 0.441 | 19.377 |
| IWS1 | | | | | | |

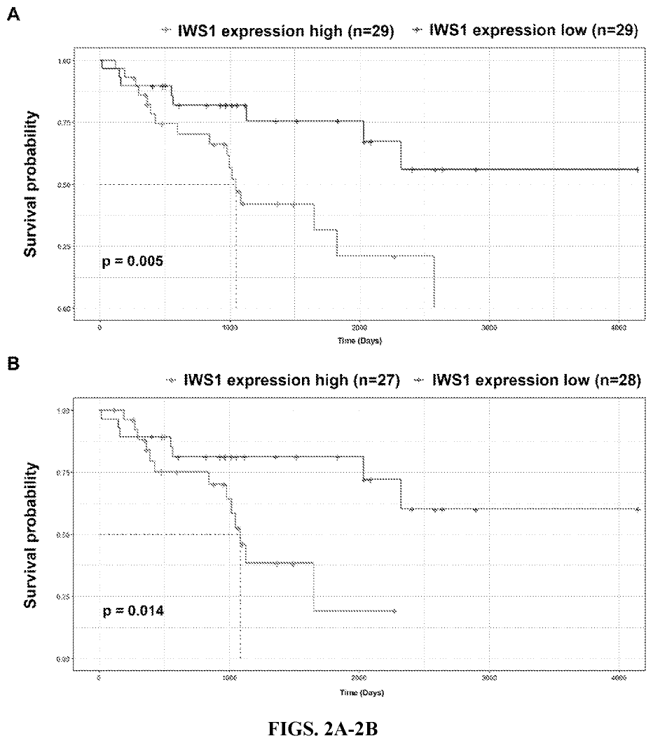

When we examined the oncologic outcomes of these patients, we found that an increase in phosphorylated IWS1 was associated with an increase in the number of LPS recurrences (Standard Error 0.112, 95% Confidence Interval 0.059-0.499, p=0.013) and a shorter disease-free survival (DFS, HR=3.652 p=0.012), but not overall survival (Tables 1-2). To further investigate this association, we carried out survival analyses based on RNA-seq data of patients with DDLPS (n=58) available from TCGA. Our analysis showed a significant reduction in overall survival (OS) and disease-specific survival (DSS) in DDLPS patients with a higher IWS1 expression compared to those with a lower IWS1 expression (OS p-value=0.005; DSS p-value=0.014). The Kaplan-Meier survival curves are shown in FIG. 2A-2B. Importantly, the impact of IWS1 expression of disease-free survival was not able to be determined since recurrence data is not currently available. In addition, RNA-Seq data from WDLPS samples are not available in TCGA so a comparison of patients with DDLPS was not possible. Nonetheless, our findings suggest that the AKT/p-IWS1 axis is active in LPS, and that increased expression of IWS1 is associated with a worse prognosis in patients.

### IWS1 Phosphorylation at Ser720/Thr721 Promotes Liposarcoma Colony Formation

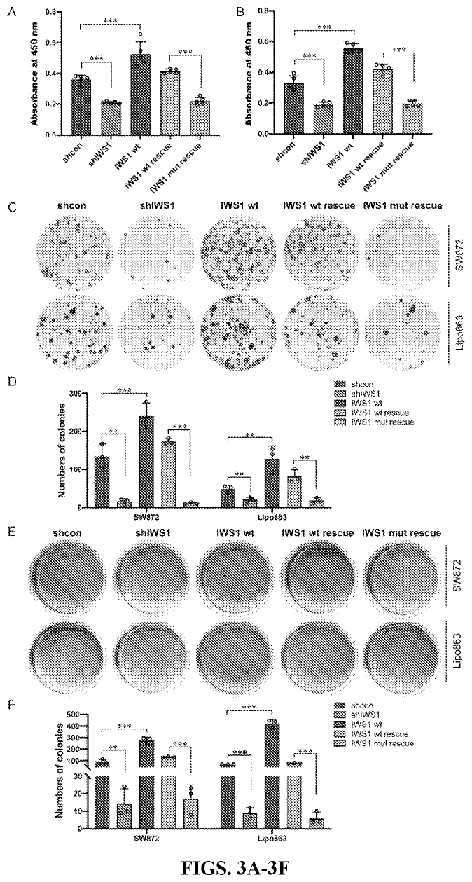

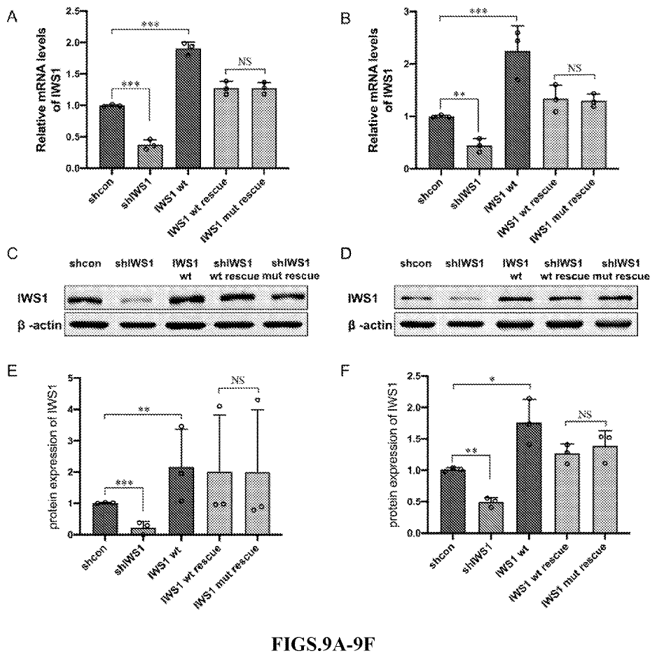

To further characterize the effect of IWS1 phosphorylation by AKT on LPS tumorigenesis, we determined the rate of colony forming capacity of LPS cells stably transduced with shControl, shIWS1, and IWS1 WT constructs. The same was done in shIWS1-transduced cells rescued with the Flag-tagged wild type IWS1 (shIWS1/WT-R) or rescued with the AKT-phosphorylation site mutant of IWS1 (Ser720A/Thr721A, shIWS1/MT-R). Notably, the ectopic Flag-tagged IWS1 clones were engineered to be shRNA-resistant (27). Using these cells, we employed the CCK8-plate colony forming assay and soft-agar colony forming assay, growing cells in complete media under normal conditions. The knockdown efficiency and ectopic Flag-IWS1 expression were confirmed with qRT-PCR and Western blotting (FIGS. 9A-9F). The loss of IWS1 expression (shIWS1) and/or phosphorylation (shIWS1/MT-R) significantly impaired plating efficiency and anchorage independent growth of Lipo863 (a dedifferentiated human LPS cell line). To determine if the observed phenotype was specific to LPS, a second experiment was performed using the undifferentiated LPS cell line, SW872 (FIGS. 3A-3F). In contrast to cells where expression of IWS1 was abrogated using shRNA, the plating efficiency and anchorage independent growth were noticeably enhanced by the increased expression of the wild type IWS1 (IWS1 WT). Importantly, the impairment of cell plating efficiency and colony formation in shIWS1 LPS cells could be rescued by wild type IWS1 (shIWS1/WT-R) but not by the mutant rescue (shIWS1/MT-R, FIGS. 3A-3F). These data indicate that IWS1 phosphorylation by AKT at Ser720/Thr721 promotes plating efficiency and colony formation of human LPS cell lines.

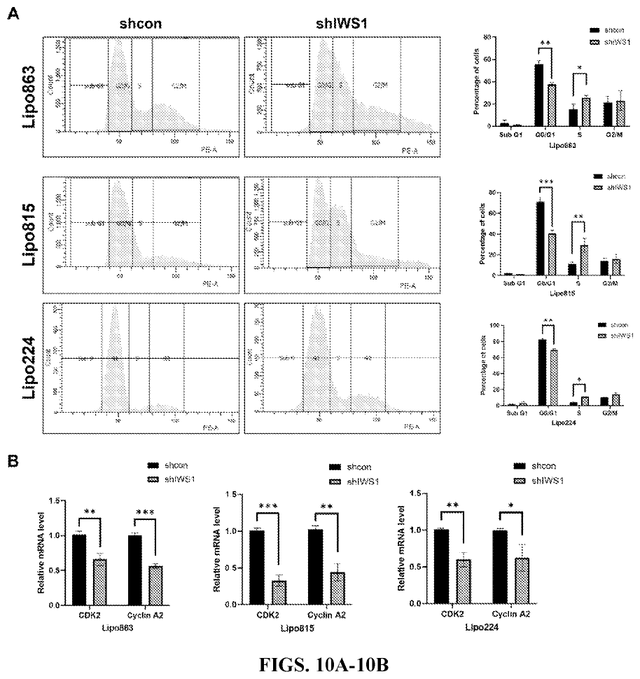

Flow cytometry using Annexin V was performed to determine if a reduction in IWS1 expression induced apoptosis and thereby reduced plating efficiency. However, there was no significant difference in shIWS1-transduced cells compared to shControl transduced cells. These findings were confirmed by performing western blot to detect the levels of cleaved caspase-3 (1:100 dilution; Cell Signaling Technology, Cat No. 9661S), protein on LPS cell lines upon IWS1 knock-down and observed no-significant difference in the expression levels of cleaved caspase-3 on shIWS1 compared to shControl cells (data not shown). To investigate how IWS1 may regulate cell growth, we used flow cytometry to characterize the impact of IWS1 expression on cell cycle progression. In three different human LPS cell lines, Lipo863, Lipo815, and Lipo224 (FIG. 10A), there was a greater proportion of cells in the S phase of the cell cycle in shIWS1 transduced cells compared to shControl cells. Noteworthy, cells knocked-down for IWS1 (Lipo863, Lipo815, and Lipo224) had lower expression of Cyclin-dependent kinase 2 (CDK2) and cyclin A2 (CCNA2) compared to the shControl cells (FIG. 10B). While the relationship between expression of IWS1, CDK2 and CCNA2 is unknown, we hypothesize that the impairment of cell growth and colony formation observed in shIWS1 cells is due to the downregulation in the expression levels of these important regulators of the cell cycle.

### IWS1 Phosphorylation at Ser720/Thr721 Promotes Liposarcoma Cell Migration and Invasion

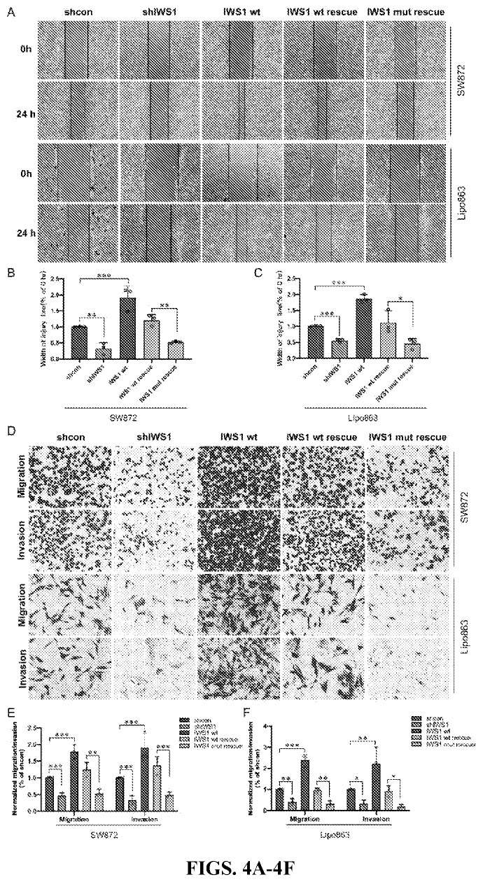

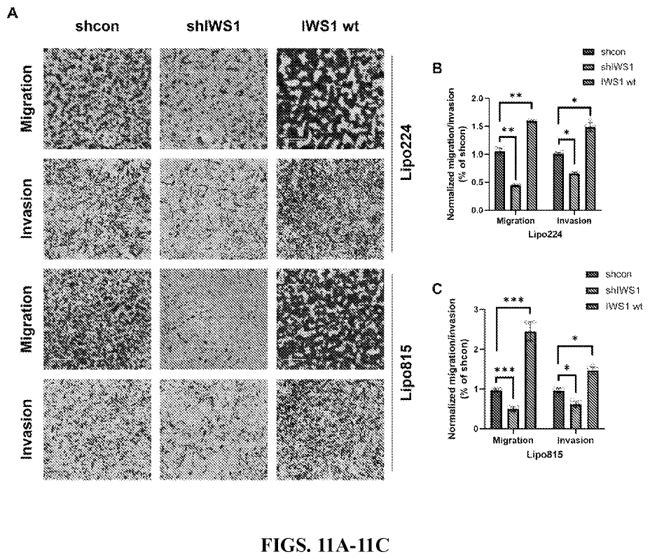

To determine whether IWS1 phosphorylation by AKT impacts the migration and invasion of LPS cells, we performed wound healing and transwell assays in SW872 and Lipo863 cells following lentiviral transduction with shControl, shIWS1, IWS1 WT, shIWS1/WT-R or shIWS1/MT-R. In addition to inhibiting the plating efficiency and anchorage-independent cell growth of LPS cells, the loss of IWS1 phosphorylation also reduced cell migration and invasion. Importantly, both cell migration and invasiveness were rescued by the wild-type IWS1 (IWS1/WT-R) but not by the phosphorylation site mutant (shIWS1/MT-R) in Lipo863 cells (FIG. 4A-4F). To determine if the observed phenotype was specific to LPS, a second experiment was performed using the undifferentiated sarcoma cell line, SW872. We also conducted migration and invasion assays on other LPS cell lines that overexpress mouse double minute 2 (MDM2), such as Lipo224 and Lipo815 cells, transduced with shControl, shIWS1, and IWS1 WT. There was a significant decrease in cell migration and invasion of both Lipo224 and Lipo815 (FIGS. 11A-11C) cells that were transduced with shIWS1 compared to the shControl cells. In contrast, the previously mentioned cell lines transduced with IWS1 WT exhibited an opposite phenotype by showing enhanced migration and invasiveness. Taken together, our findings clearly demonstrate the impact of IWS1 phosphorylation at Ser720/Thr721 by AKT in cell migration and invasion of LPS.

### IWS1 Phosphorylation Ser720/Thr721 Promotes Tumor Growth and Metastasis of Liposarcoma In Vivo

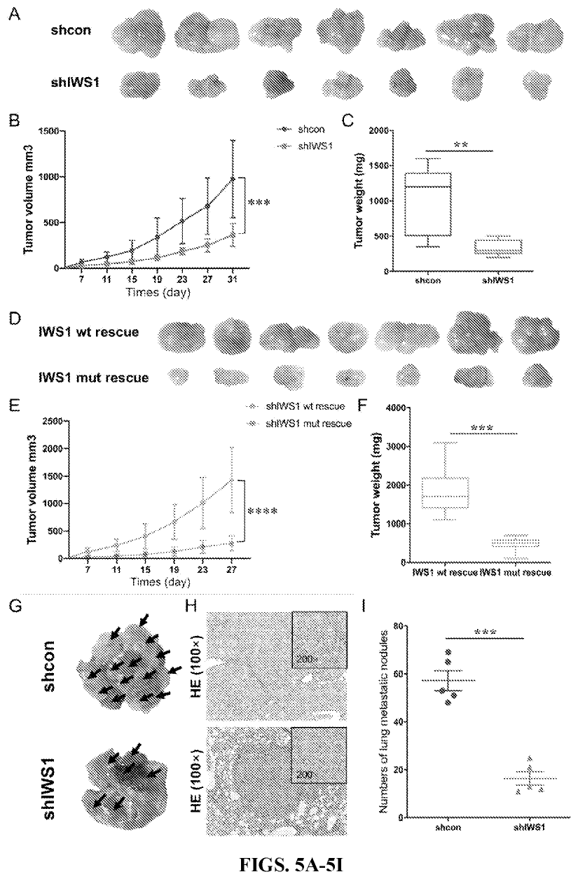

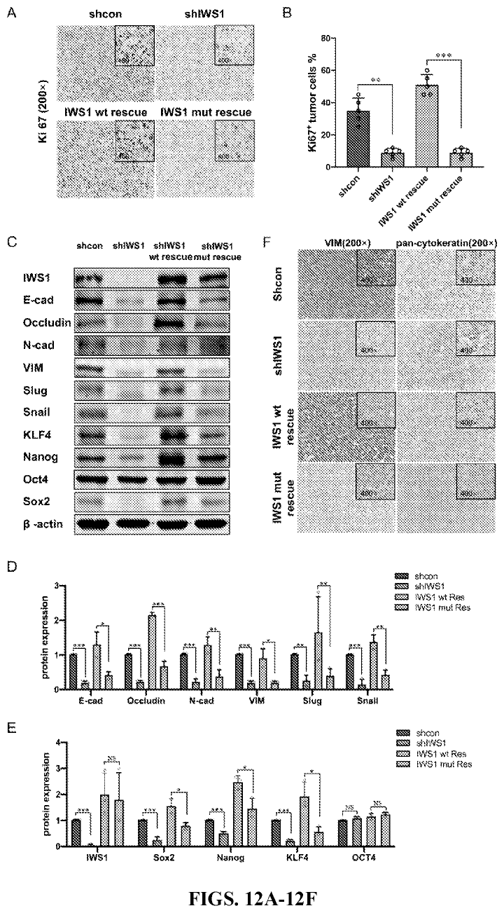

To address the role of IWS1 phosphorylation in sarcoma tumor growth in vivo, we injected 5×10⁶ shControl, shIWS1, shIWS1/WT-R or shIWS1/MT-R SW872 cells in immunocompromised NSG mice. Cells were injected either subcutaneously (sc) or intravenously (IV-tail vein). The animals were monitored for tumor development and lung metastasis for 4 and 6 weeks, respectively. Consistent with our in vitro data, the results revealed that the knockdown of IWS1 resulted in reduced tumor growth (tumor volume and weight) and expression of Ki 67, with a parallel reduction in the number of metastatic lung nodules (FIGS. 5A-5C, 5G-5I and FIGS. 12A-12B). Importantly, the observed reduction in tumor growth, expression of Ki 67, and lung metastases were rescued in the shIWS1/WT-R tumor bearing animals, but not in the shIWS1/MT-R (FIGS. 5D-5F and FIG. 12A-12B). The knockdown of IWS1 and its rescue by the wild type and mutant IWS1 were validated by western blotting of human cell lysates (FIG. 12C).

Western blots of the same cell lysates also showed that the knockdown of IWS1 resulted in the downregulation of the EMT markers E-Cadherin, Occludin, N-Cadherin, and Vimentin, the EMT promoting factors SNAIL and SLUG, the stemness factors FLF3, Nanog, OCT4 and Sox2. Moreover, whereas the wild type IWS1 rescued the expression of these proteins, the mutant IWS1 failed to do so (FIG. 12C). Downregulation of Vimentin, along with the expression of cytokeratin, which is also downregulated in shIWS1 and shIWS1/MT-R xenografts, were also examined by immunohistochemistry (FIG. 5D).

### IWS1 Phosphorylation at Ser720/Thr721 Promotes a Mesenchymal-to-Epithelial Plasticity and Cancer Stem Cell-Renewal Program in Human Liposarcoma Cell Lines

The PIK3/AKT pathway has been associated with EMT in several human cancers, through direct phosphorylation, or indirectly, through transcriptional regulation of EMT effectors (40-42). Recent evidence suggests that EMT is reversible, and a strong link exists between EMT/MET and CSC formation (12, 15). EMT/MET programs confer CSC-like characteristics to cells within the tumor and are important drivers of tumoral heterogeneity and resistance to currently available therapies (17, 18, 43). As such, we examined whether the loss of IWS1 or phosphorylated IWS1 alters the expression of EMT markers and transcription factors association with EMT.

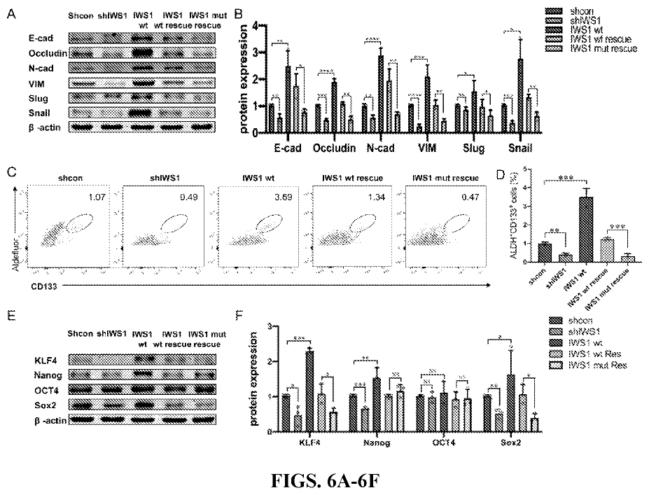

Occludin and E-cadherin are markers of epithelial cells and are downregulated in EMT. N-Cadherin and Vimentin are markers of mesenchymal cells and are induced in cells undergoing EMT. The Western blot in FIGS. 6A-6B shows that knockdown of IWS1 downregulates both E-Cadherin and Occludin, as well as N-Cadherin and Vimentin. This suggests IWS1 inhibits both the MET and EMT program in the undifferentiated LPS cell line SW872, thereby maintaining plasticity and tumoral heterogeneity. In further support, the overexpression of IWS1 gave rise to the opposite phenotype. Importantly, the phosphorylation site IWS1 mutant failed to rescue the phenotype of the IWS1 knockdown. This suggests that the downregulation of these markers is IWS1-phosphorylation dependent and suggest that IWS1 phosphorylation may promote both the MET and the EMT transition resulting in a population of “metastable” cells. To this end, the results in FIGS. 6A-6B also shows that the expression of the EMT-promoting transcription factors Snail and Slug follow the same pattern as the expression of the MET and EMT markers upon the knockdown or overexpression of IWS1 in these same cell lines.

Cells undergoing EMT tend to exhibit stem cell properties (16). This, combined with the observation that AKT signaling has been linked to maintenance of stem cells in LPS and that IWS1 has been shown to regulate the expression of Nanog, a stemness promoting factor, raised the question of whether IWS1 regulates the stemness of LPS cell lines (22, 28). To address these questions, we used flow cytometry to determine whether the knockdown of IWS1 and its rescue by the phosphorylation site IWS1 mutant affected the relative number of cells expressing the LPS stem cell markers ALDH and CD133.(9, 10) Our results showed that that a small percentage of shControl SW872 cells (0.92%-1.15%) display high CD133 expression and ALDH activity (FIGS. 6C-6D). Importantly, the reduced percentage of stem cells was rescued in shIWS1/WT-R, but not in shIWS1/MT-R cells (FIGS. 6C-6D). Next, Western blotting was performed to determine whether the knockdown of IWS1 and its rescue by the phosphorylation site IWS1 mutant affected the stemness promoting transcription factors KLF4, Nanog, OCT4, and Sox2 in SW872 cells (FIGS. 6E-6F). These data indicate that the IWS1 phosphorylation at Ser720/Thr721 by AKT kinase promotes EMT/MET plasticity and cancer stem cell-like properties in LPS cells.

### DISCUSSION

In this example, we demonstrate that phosphorylation of the transcription elongation factor, IWS1, by AKT is an important cellular event that maintains CSCs and mesenchymal-epithelial plasticity in human LPS. Specifically, phosphorylated IWS1 maintains levels of Nanog, the presence of CD133+ CSCs and EMT status in both cell and xenograft models of LPS and is required for anchorage-independent growth, migration, invasion, and tumor metastases. In patients with LPS, IWS1 expression is associated with a reduction in overall survival, and the presence of phosphorylated IWS1 is associated with an increase in the number of tumor recurrences and a shorter time to relapse.

Mechanistically, phosphorylation of IWS1 by AKT3 and AKT1 at Ser720/Thr721 recruits the Histone H3K36 trimethyl-transferase, SETD2, to the RNA Poll II complex. Together these proteins create a docking site for MRG15 and PTB that is required for transcription elongation (27). This study confirms a similar role of PI3K/AKT signaling and IWS1 phosphorylation in the cell biology and tumorigenesis of human LPS.

Dysregulation of PI3K/AKT signaling has long been implicated in the aggressive clinical behavior of LPS and other types of soft tissue sarcoma (20, 21). We found that phosphorylation of the transcription elongation factor, IWS1, by AKT is an important cellular event required for anchorage-independent growth, migration, invasion, and in vivo tumor metastases in preclinical models of human LPS. Moreover, the results are consistent in undifferentiated cell lines of LPS and in those with MDM2 amplification, suggesting that the impact of IWS1 on LPS phenotype is independent of MDM2. In addition, a retrospective analysis of patients treated at our center found that increased levels of phosphorylated IWS1 in resected tumors was associated with an increase in the number of tumor recurrences and a shorter disease-free interval in patients with retroperitoneal LPS. This finding is consistent with previous studies on AKT in LPS but is the first to implicate the transcription elongation factor IWS1 and its phosphorylation by AKT as a critical event in maintaining an aggressive phenotype.

LPS originate from the mesenchymal stem cell population, and it is well known that these CSCs are maintained as tumors progress. In fact, grade 4 dedifferentiated LPS express stem cell markers (44). It is believed that these CSCs confer resistance to chemo and radiotherapy that is so characteristic of LPS (23). In our study, we found that the phosphorylation of IWS1 maintains Nanog expression and CD133+ CSC phenotypes in human LPS cell lines and xenograft models. Nanog is an important molecule for tumor growth and progression in sarcomas and confers resistance to Doxorubicin (22, 45). Consistently, we observed that IWS1 phosphorylation promotes tumor growth and lung metastasis in vivo. These results are consistent with previous separate reports in the literature, whereby AKT kinase maintains Nanog expression and CSCs in sarcoma, and where phosphorylation of IWS1 is necessary for Nanog expression in murine embryonic development (22, 28). The results of this study suggest that IWS1 phosphorylation by AKT is an important event for maintaining Nanog expression in human LPS and based on recent findings where inhibition of CSCs by targeting AKT reduced chemo and radio resistance, may hold potential as a novel therapeutic target. Targeting IWS1 specifically may circumvent the toxicity of currently available AKT inhibitors, while reducing resistance to chemo and/or radiotherapy by reversing CSC phenotypes in patients with LPS and other forms of sarcoma (46-48). On the other hand, since IWS1 is ubiquitously expressed, its inhibition may lead to significant toxicity (39).

Increased cell migration and invasiveness in epithelial cell-derived tumors is commonly the result of EMT. During EMT cells lose the epithelial phenotype and become more migratory and invasive by acquiring a mesenchymal phenotype (16). Recent studies have shown that this is a multifaceted process rather than a single, linear event and is implicated in cancer progression, metastasis, and drug resistance (15, 16). Importantly, this process is reversible and tumor cells can undergo MET whereby they acquire a more sessile and proliferative phenotype like that of epithelial cells (12). Sarcomas are characterized by their mesenchymal features. As such, EMT in sarcomas may seem like a paradox (12). However, many sarcomas are heterogeneous, with many of their cells having an intermediate phenotype that express both epithelial and mesenchymal markers (13, 49-51). The presence of cells exhibiting this intermediate “meta stable” phenotype is considered a feature of tumors with more aggressive tumor biology because these cells can undergo MET and give rise to highly proliferative cells in the primary tumor, or EMT, giving rise to migratory, invasive, and metastatic cells (12).

In this example, we found that the knockdown of IWS1 downregulates both E-Cadherin and Occludin, as well as N-Cadherin and Vimentin, suggesting that it inhibits both the MET and the EMT program in the undifferentiated LPS cell line SW872. More so, overexpression of IWS1 gave rise to the opposite phenotype, supporting the conclusion that IWS1 may promote a “metastable” population of cells. Importantly, the phosphorylation site IWS1 mutant failed to rescue the phenotype of the IWS1 knockdown and suggests that the downregulation of these markers is IWS1-phosphorylation dependent. In further support, the expression of the EMT-promoting transcription factors Snail and Slug, both of which are canonically downstream of PI3K/AKT, followed the same pattern of expression as the MET/EMT markers upon knockdown or overexpression of IWS1 in these same cell lines. Lastly, phosphorylation of IWS1 was required for anchorage-independent growth, migration and invasion, and tumor metastases. These findings are the first to implicate AKT-dependent phosphorylation of the transcription elongation factor IWS1 in the maintenance of this “metastable” phenotype and increased tumorigenesis in cancer.

While the exact mechanism of how IWS1 exerts its effects on LPS cells is unknown, our results also suggest that phosphorylation of IWS1 by AKT may play a role in cell cycle progression. AKT is an important regulator of the cell cycle by maintaining protein stability of during multiple cell cycle checkpoints (52). We found that knock-down of the AKT-target, IWS1, resulted in a down-regulation of CDK2 and cyclin A2 expression and lead to a greater proportion of cells in the S phase. By altering cell cycle dynamics, IWS1 may impact cell viability, colony formation, cell migration and invasion.

In conclusion, the phosphorylation of IWS1 by AKT is an important cellular event that maintains tumoral heterogeneity and the oncogenic potential of human LPS by maintaining CSCs and EMT/MET plasticity. These findings support the use of the AKT/IWS1 axis as a novel prognostic factor and potential therapeutic target in patients with advanced LPS.

### REFERENCES

1. Janeway K A, Maki R G. New strategies in sarcoma therapy: linking biology and novel agents. Clin Cancer Res. 2012; 18(21):5837-44.
2. Chen J, Hang Y, Gao Q, Huang X. Surgical Diagnosis and Treatment of Primary Retroperitoneal Liposarcoma. Front Surg. 2021; 8:672669.
3. Strauss D C, Hayes A J, Thomas J M. Retroperitoneal tumours: review of management. Ann R Coll Surg Engl. 2011; 93(4):275-80.
4. Haddox C L, Riedel R F. Recent advances in the understanding and management of liposarcoma. Fac Rev. 2021; 10:1.
5. Lee A T J, Thway K, Huang P H, Jones R L. Clinical and Molecular Spectrum of Liposarcoma. J Clin Oncol. 2018; 36(2):151-9.
6. Livingston J A, Bugano D, Barbo A, Lin H, Madewell J E, Wang W L, et al. Role of chemotherapy in dedifferentiated liposarcoma of the retroperitoneum: defining the benefit and challenges of the standard. Sci Rep. 2017; 7(1):11836.
7. Trans-Atlantic Retroperitoneal Sarcoma Working Group. Electronic address ambbc. Management of metastatic retroperitoneal sarcoma: a consensus approach from the Trans-Atlantic Retroperitoneal Sarcoma Working Group (TARPSWG). Ann Oncol. 2018; 29(4):857-71.
8. Tyler R, Wanigasooriya K, Taniere P, Almond M, Ford S, Desai A, et al. A review of retroperitoneal liposarcoma genomics. Cancer Treat Rev. 2020; 86:102013.
9. Genadry K C, Pietrobono S, Rota R, Linardic C M. Soft Tissue Sarcoma Cancer Stem Cells: An Overview. Front Oncol. 2018; 8:475.
10. Martinez-Delgado P, Lacerenza S, Obrador-Hevia A, Lopez-Alvarez M, Mondaza-Hernandez J L, Blanco-Alcaina E, et al. Cancer Stem Cells in Soft-Tissue Sarcomas. Cells. 2020; 9(6).
11. Zhang Y, Young E D, Bill K, Belousov R, Peng T, Lazar A J, et al. Heterogeneity and immunophenotypic plasticity of malignant cells in human liposarcomas. Stem Cell Res. 2013; 11(2):772-81.
12. Sannino G, Marchetto A, Kirchner T, Grunewald T G P. Epithelial-to-Mesenchymal and Mesenchymal-to-Epithelial Transition in Mesenchymal Tumors: A Paradox in Sarcomas?Cancer Res. 2017; 77(17):4556-61.
13. Yang J, Du X, Wang G, Sun Y, Chen K, Zhu X, et al. Mesenchymal to epithelial transition in sarcomas. Eur J Cancer. 2014; 50(3):593-601.
14. Scheel C, Weinberg R A. Cancer stem cells and epithelial-mesenchymal transition: concepts and molecular links. Semin Cancer Biol. 2012; 22(5-6):396-403.
15. Sato R, Semba T, Saya H, Arima Y. Concise Review: Stem Cells and Epithelial-Mesenchymal Transition in Cancer: Biological Implications and Therapeutic Targets. Stem Cells. 2016; 34(8):1997-2007.
16. Lambert A W, Weinberg R A. Linking EMT programmes to normal and neoplastic epithelial stem cells. Nat Rev Cancer. 2021; 21(5):325-38.
17. Deng J, Wang L, Chen H, Hao J, Ni J, Chang L, et al. Targeting epithelial-mesenchymal transition and cancer stem cells for chemoresistant ovarian cancer. Oncotarget. 2016; 7(34):55771-88.
18. Ghosh R D, Ghuwalewala S, Das P, Mandloi S, Alam S K, Chakraborty J, et al. MicroRNA profiling of cisplatin-resistant oral squamous cell carcinoma cell lines enriched with cancer-stem-cell-like and epithelial-mesenchymal transition-type features. Sci Rep. 2016; 6:23932.
19. Su F, Ahn S, Saha A, DiGiovanni J, Kolonin M G. Adipose stromal cell targeting suppresses prostate cancer epithelial-mesenchymal transition and chemoresistance. Oncogene. 2019; 38(11):1979-88.
20. Gutierrez A, Snyder E L, Marino-Enriquez A, Zhang Y X, Sioletic S, Kozakewich E, et al. Aberrant A K T activation drives well-differentiated liposarcoma. Proc Natl Acad Sci USA. 2011; 108(39):16386-91.
21. Ishii T, Kohashi K, Iura K, Maekawa A, Bekki H, Yamada Y, et al. Activation of the Akt-mTOR and MAPK pathways in dedifferentiated liposarcomas. Tumour Biol. 2016; 37(4):4767-76.
22. Yoon C, Lu J, Yi B C, Chang K K, Simon M C, Ryeom S, et al. PI3K/Akt pathway and Nanog maintain cancer stem cells in sarcomas. Oncogenesis. 2021; 10(1):12.
23. Xiao W, Mohseny A B, Hogendoorn P C, Cleton-Jansen A M. Mesenchymal stem cell transformation and sarcoma genesis. Clin Sarcoma Res. 2013; 3(1):10.
24. Chang L, Graham P H, Hao J, Ni J, Bucci J, Cozzi P J, et al. Acquisition of epithelial-mesenchymal transition and cancer stem cell phenotypes is associated with activation of the PI3K/Akt/mTOR pathway in prostate cancer radioresistance. Cell Death Dis. 2013; 4:e875.
25. Bakin A V, Tomlinson A K, Bhowmick N A, Moses H L, Arteaga C L. Phosphatidylinositol 3-kinase function is required for transforming growth factor beta-mediated epithelial to mesenchymal transition and cell migration. J Biol Chem. 2000; 275(47):36803-10.
26. Xu Q, Ma J, Lei J, Duan W, Sheng L, Chen X, et al. alpha-Mangostin suppresses the viability and epithelial-mesenchymal transition of pancreatic cancer cells by downregulating the PI3K/Akt pathway. Biomed Res Int. 2014; 2014:546353.
27. Sanidas I, Polytarchou C, Hatziapostolou M, Ezell S A, Kottakis F, Hu L, et al. Phosphoproteomics screen reveals akt isoform-specific signals linking RNA processing to lung cancer. Mol Cell. 2014; 53(4):577-90.
28. Oqani R K, Lin T, Lee J E, Kang J W, Shin H Y, Il Jin D. Iwsl and Spt6 Regulate Trimethylation of Histone H3 on Lysine 36 through Akt Signaling and are Essential for Mouse Embryonic Genome Activation. Sci Rep. 2019; 9(1):3831.
29. Yoh S M, Cho H, Pickle L, Evans R M, Jones K A. The Spt6 SH2 domain binds Ser2-P RNAPII to direct Iws1-dependent mRNA splicing and export. Genes Dev. 2007; 21(2):160-74.
30. Yoh S M, Lucas J S, Jones K A. The Iws1:Spt6:CTD complex controls cotranscriptional mRNA biosynthesis and HYPB/Setd2-mediated histone H3K36 methylation. Genes Dev. 2008; 22(24):3422-34.
31. Dong X, Chen R. Understanding aberrant RNA splicing to facilitate cancer diagnosis and therapy. Oncogene. 2020; 39(11):2231-42.
32. Core L J, Waterfall J J, Lis J T. Nascent RNA sequencing reveals widespread pausing and divergent initiation at human promoters. Science. 2008; 322(5909):1845-8.
33. Mayer A, di Iulio J, Maleri S, Eser U, Vierstra J, Reynolds A, et al. Native elongating transcript sequencing reveals human transcriptional activity at nucleotide resolution. Cell. 2015; 161(3):541-54.
34. Rapp T B, Yang L, Conrad E U 3rd, Mandahl N, Chansky H A. RNA splicing mediated by YB-1 is inhibited by TLS/CHOP in human myxoid liposarcoma cells. J Orthop Res. 2002; 20(4):723-9.
35. Bonnal S C, Lopez-Oreja I, Valcarcel J. Roles and mechanisms of alternative splicing in cancer—implications for care. Nat Rev Clin Oncol. 2020; 17(8):457-74.
36. Huang J, Sachdeva M, Xu E, Robinson T J, Luo L, Ma Y, et al. The Long Noncoding RNA NEAT1 Promotes Sarcoma Metastasis by Regulating RNA Splicing Pathways. Mol Cancer Res. 2020; 18(10):1534-44.
37. Karni R, de Stanchina E, Lowe S W, Sinha R, Mu D, Krainer A R. The gene encoding the splicing factor SF2/ASF is a proto-oncogene. Nat Struct Mol Biol. 2007; 14(3):185-93.
38. Miller T E, Liau B B, Wallace L C, Morton A R, Xie Q, Dixit D, et al. Transcription elongation factors represent in vivo cancer dependencies in glioblastoma. Nature. 2017; 547(7663):355-9.
39. Orlacchio A, Stark A E, Foray C, Amari F, Sheetz T, Reese E, et al. Genetic ablation of interacting with Spt6 (Iwsl) causes early embryonic lethality. PLoS One. 2018; 13(9):e0201030.
40. Xue G, Restuccia D F, Lan Q, Hynx D, Dirnhofer S, Hess D, et al. Akt/PKB-mediated phosphorylation of Twist1 promotes tumor metastasis via mediating cross-talk between PI3K/Akt and TGF-beta signaling axes. Cancer Discov. 2012; 2(3):248-59.
41. Xu W, Yang Z, Lu N. A new role for the PI3K/Akt signaling pathway in the epithelial-mesenchymal transition. Cell Adh Migr. 2015; 9(4):317-24.
42. Tian H, Lian R, Li Y, Liu C, Liang S, Li W, et al. AKT-induced incRNA VAL promotes EMT-independent metastasis through diminishing Trim16-dependent Vimentin degradation. Nat Commun. 2020; 11(1):5127.
43. Gooding A J, Schiemann W P. Epithelial-Mesenchymal Transition Programs and Cancer Stem Cell Phenotypes: Mediators of Breast Cancer Therapy Resistance. Mol Cancer Res. 2020; 18(9):1257-70.
44. Stratford E W, Castro R, Wennerstrom A, Holm R, Munthe E, Lauvrak S, et al. Liposarcoma Cells with Aldefluor and CD133 Activity have a Cancer Stem Cell Potential. Clin Sarcoma Res. 2011; 1(1):8.
45. Yoon C, Chang K K, Lee J H, Tap W D, Hart C P, Simon M C, et al. Multimodal targeting of tumor vasculature and cancer stem-like cells in sarcomas with VEGF-A inhibition, HIF-lalpha inhibition, and hypoxia-activated chemotherapy. Oncotarget. 2016; 7(28):42844-58.
46. Zhu Q S, Ren W, Korchin B, Lahat G, Dicker A, Lu Y, et al. Soft tissue sarcoma cells are highly sensitive to AKT blockade: a role for p53-independent up-regulation of GADD45 alpha. Cancer Res. 2008; 68(8):2895-903.
47. Patel S. Exploring novel therapeutic targets in GIST: focus on the PI3K/Akt/mTOR pathway. Curr Oncol Rep. 2013; 15(4):386-95.
48. Setsu N, Kohashi K, Fushimi F, Endo M, Yamamoto H, Takahashi Y, et al. Prognostic impact of the activation status of the Akt/mTOR pathway in synovial sarcoma. Cancer. 2013; 119(19):3504-13.
49. Qi Y, Wang N, He Y, Zhang J, Zou H, Zhang W, et al. Transforming growth factor-betal signaling promotes epithelial-mesenchymal transition-like phenomena, cell motility, and cell invasion in synovial sarcoma cells. PLoS One. 2017; 12(8):e0182680.
50. Damerell V, Pepper M S, Prince S. Molecular mechanisms underpinning sarcomas and implications for current and future therapy. Signal Transduct Target Ther. 2021; 6(1):246.
51. Fitzgerald M P, Gourronc F, Teoh M L, Provenzano M J, Case A J, Martin J A, et al. Human Chondrosarcoma Cells Acquire an Epithelial-Like Gene Expression Pattern via an Epigenetic Switch: Evidence for Mesenchymal-Epithelial Transition during Sarcomagenesis. Sarcoma. 2011; 2011:598218.
52. Chang F, Lee J T, Navolanic P M, Steelman L S, Shelton J G, Blalock W L, et al. Involvement of PI3K/Akt pathway in cell cycle progression, apoptosis, and neoplastic transformation: a target for cancer chemotherapy. Leukemia. 2003; 17(3):590-603.
53. Peng T, Zhang P, Liu J, Nguyen T, Bolshakov S, Belousov R, et al. An experimental model for the study of well-differentiated and dedifferentiated liposarcoma; deregulation of targetable tyrosine kinase receptors. Lab Invest. 2011; 91(3):392-403.
54. Robinson M D, McCarthy D J, Smyth G K. edgeR: a Bioconductor package for differential expression analysis of digital gene expression data. Bioinformatics. 2010; 26(1):139-40.

### Example 2. Evaluation of Small Molecules for the Treatment of Lipocarcinoma

In this Example, we identify small molecules that can be used as a treatment for patients with liposarcoma (LPS). We have shown that these compounds can act as inhibitors of the transcription elongation factor, IWS1 (Uniprot ID: Q96ST2). When IWS1 is inhibited by these compounds, the spread and progression of liposarcoma can be reduced.

IWS1 is upregulated in human liposarcoma compared to adjacent adipose tissue. Moreover, IWS1 expression is associated with worse survival and shorter time to relapse. The activation of IWS1 (ie. phosphorylated AKT) maintains levels of Nanog, the presence of CD133+ cancer stem cells, and epithelial to mesenchymal status in both cell and xenograft models of liposarcoma (markers of chemo and radioresistance and tumor aggressiveness). In addition, IWS1 is required for anchorage-independent growth, migration, invasion, and in vivo tumor metastases in preclinical models of human LPS (markers of aggressiveness). Commercially available inhibitors of AKT (upstream of IWS1) have been used in early phase clinical studies but are toxic and have not been approved due to their lack of efficacy and poor tolerability. Targeting IWS1 may circumvent this problem: it can reduce resistance to chemotherapy by reversing CSC phenotypes as well as reduce the metastatic potential of the tumor cells. The development of a pharmacological IWS1 inhibitor can lead to the establishment of a new efficient combination therapy for liposarcoma.

Specifically, we have identified IWS1 inhibitors that reduce the binding of IWS1 and Spt6 (Uniprot ID: Q7KZ85). The formation of the IWS1/Spt6 complex is required for the formation of the RNAPII complex, the machinery that transcribes DNA into mRNA precursors for a variety of biological activities. Evidence from mutagenesis studies indicates that mutations of key residues at the IWS1/Spt6 interface disrupt the complex formation, and consequently, down-regulate the transcriptional activities of RNAPII and alter specific pathways, including those important to tumor progression [1]. The presence of IWS1 alone, however, downregulates part of the transcriptional activities, based on a genetic screen indicating that IWS1 has a strong antagonizing effect towards the chromatin remodeling complex Swi/Snf [2]. In some cases, the inhibitors can target the IWS1/Spt6 interface, and can not interfere with other domains of IWS1. Consequently, reducing Spt6 interaction in a competitive manner without abolishing the other regulatory roles of IWS1 shall result in a unidirectional reduction of transcriptional activities. Therefore, the abrogation of the IWS1/Spt6 complex will reduce specific transcriptional activities such as growth, proliferation, and migration of the tumor cells.

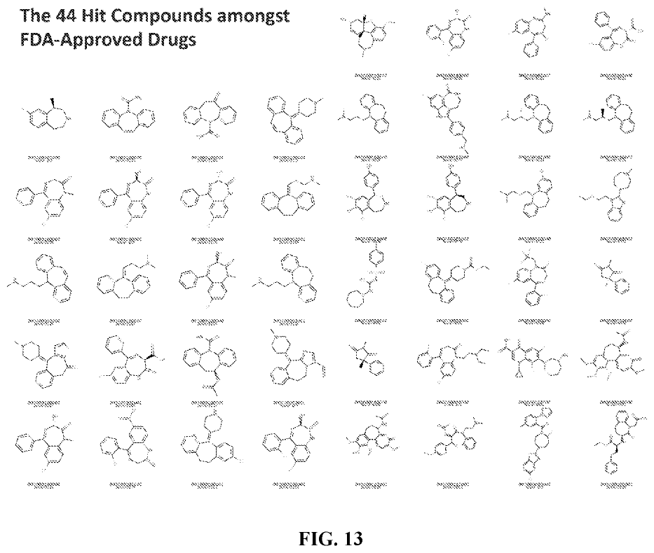

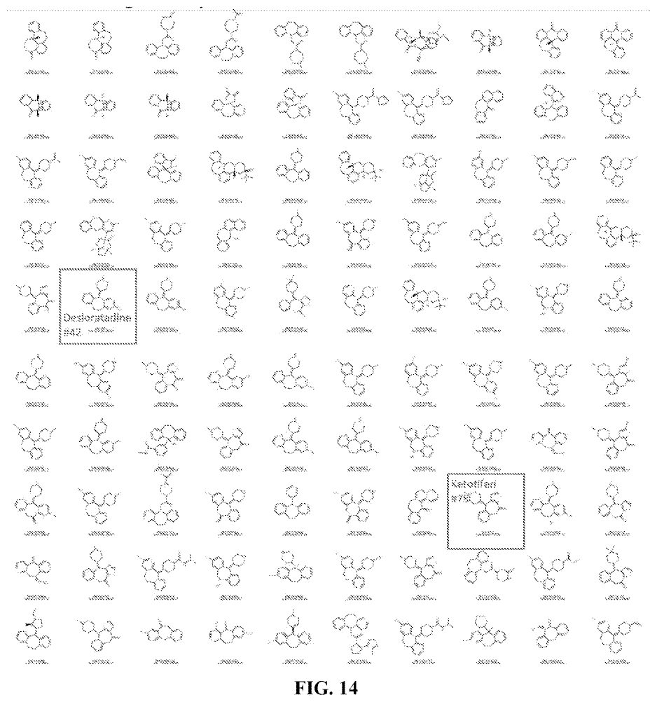

In some cases, the IWS1 inhibitors identified herein can share similar structural features, including a tricyclic fused-ring structure. The composition of the tricyclic fused-ring system features a central seven-membered ring that is cycloheptane or cycloheptene, with two ortho-fused aromatic rings that are six- or five-membered. The class of compounds includes known compounds, such as the FDA-approved drugs Ketotifen, Desloratadine, and Cyproheptadine. See FIG. 13. No preferred chirality or stereochemistry has been established. The class of compounds also includes commercially available compounds containing the aforementioned chemical pattern as a substructure. A list of such compounds is included in FIG. 14. This class of compounds was initially identified as hypothetically potent inhibitors by computer-aided, structure-based virtual screening approaches.

In the first round of virtual screening, a subset of FDA-approved drugs was evaluated by molecular docking calculations, and the top-ranked compounds were selected based on the calculated theoretical potency. A human IWS1 structure was retrieved from the AlphaFold database, a library of predicted protein structures by the AI-based algorithm [3]. A putative binding site was defined on IWS1 featuring a concave-shaped pocket that is necessary to accommodate a key phenylalanine residue of Spt6 in the IWS1/Spt6 complex. The protein structure serves as the structural basis for the following in silico compound screening, where a theoretical potency is computed per compound using the Vina scoring function [4]. The best-scored binding poses were predicted by the docking program AutoDock Vina, and the best-reported score per compound was then used as the major descriptor for compound selection. This initial virtual screening provides a selection of strong candidates amongst the FDA-approved drugs as inhibitors for IWS1 targeting the IWS1/Spt6 interface. These known drugs, including Ketotifen, Desloratadine, and Cyproheptadine, can be repurposed as a treatment for patients with liposarcoma.

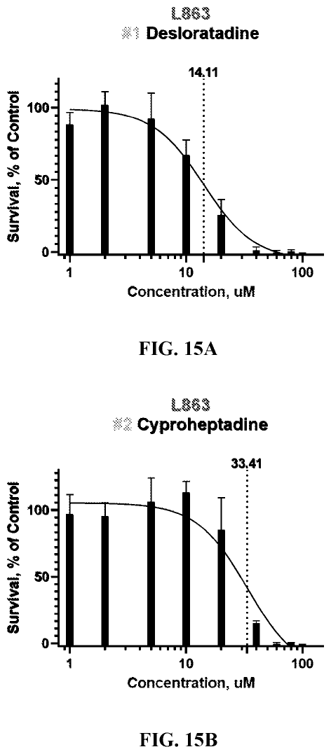

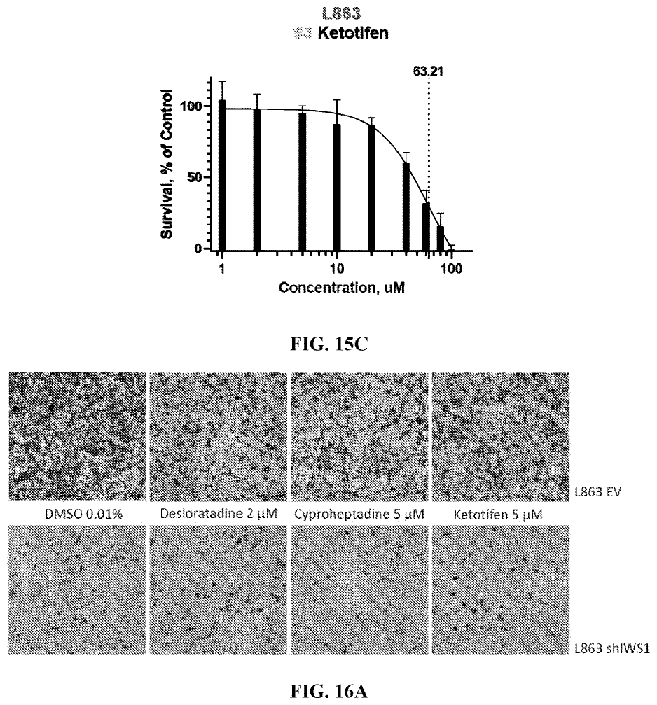

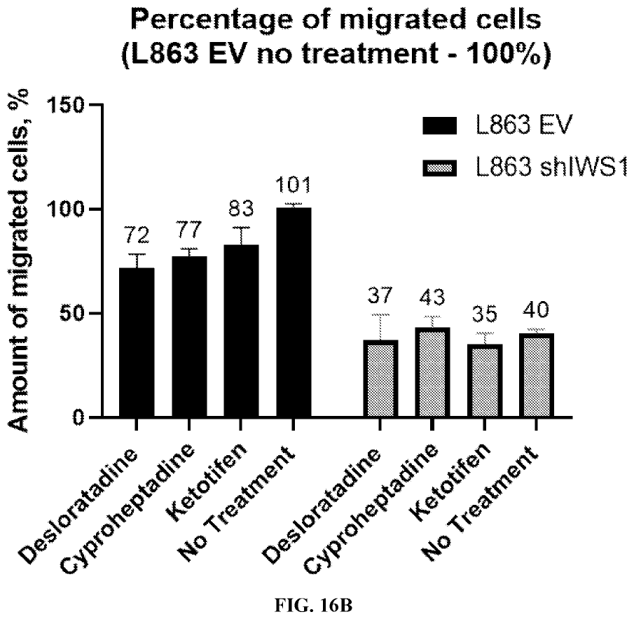

To evaluate these compounds, we used dedifferentiated cell lines Lipo863 and Lipo815 as an in-vitro model of dedifferentiated liposarcoma (DDLPS). Using a cell cytotoxicity assay, we determined the cytotoxicity of Ketotifen, Desloratadine, and Cyproheptadine. The IC50 values for Ketotifen, Desloratadine, and Cyproheptadine were 63, 14, and 33 uM, accordingly. FIG. 15A-15C. Using these candidates as potential pharmacological inhibitors of IWS1, we expected to reproduce/mimic phenotypical changes specific for lentiviral IWS1 knockdown. Particularly, as described in Example 1, we demonstrated a significant decrease in the migratory ability of cells after IWS1 knockdown [8]. We performed a migration assay to assess the ability of cells to migrate after 24 hours of treatment compared to intact cells (EV) as well as IWS1 knockdown cells (shIWS1). See FIG. 16A-16B. Using drug concentrations that would not affect the viability of the cells, we sought to evaluate predominantly the migratory ability of cells. All 3 drugs showed a reduction in cell migration compared to the control group: Desloratadine−29%, Cyproheptadine−24%, and Ketotifen −18%. However, there was no significant reduction in migration for shIWS1 cells (comparing treated groups to the non-treatment control). In summary, the reduction in the migratory ability of cells induced by drug treatment was dependent on the presence of IWS1. Moreover, the effect on migration was dose-dependent: 1-10 uM solutions of Ketotifen decreased migration ability by 18-44%, and 0.4-4 uM solutions of Desloratadine decreased migration ability by 34-53%. FIG. 17. Dose-dependent effect of drugs on the migratory ability of cells suggests that there may be an optimal in vivo concentration to slow down or even stop cells from metastasizing from the primary tumor to distant organs like the lung or liver.

To demonstrate the ability of this compound to disrupt the IWS1/spt6 binding, we performed a co-immunoprecipitation experiment where the native complex of two or more proteins is isolated and analyzed. We performed Co-IP experiments after 48 hours of treatment with Ketotifen, Desloratadine, and DMSO (control) and analyzed the amount of the complex pulled down from the cells. The results showed a decreased amount of complex presented in the cells by more than 50% after treatment with Ketotifen/Desloratadine compared to the DMSO negative control. The Co-IP method allowed us to analyze IWS1/Spt6 protein complex directly and assess the efficiency of the drug candidates to disrupt the complex.

The binding of the small molecule to IWS1 may cause the redistribution of the latter in the cell (cytoplasmic vs nuclear locations) due to conformational changes of the protein or disruption of its functions. To characterize the effect of a drug candidate on the distribution of IWS1 in the cell, we performed fraction analysis by separating cytoplasmic and nuclear proteins. After a 48-hour treatment, the ratio between nuclear and cytosolic fractions of IWS1 increased by 1.1 for 20 uM Ketotifen and by 3.3 for 5 uM Desloratadine compared to DMSO negative control with a ratio of 0.75. This suggests IWS1 is accumulated in the nucleus in response to treatment.

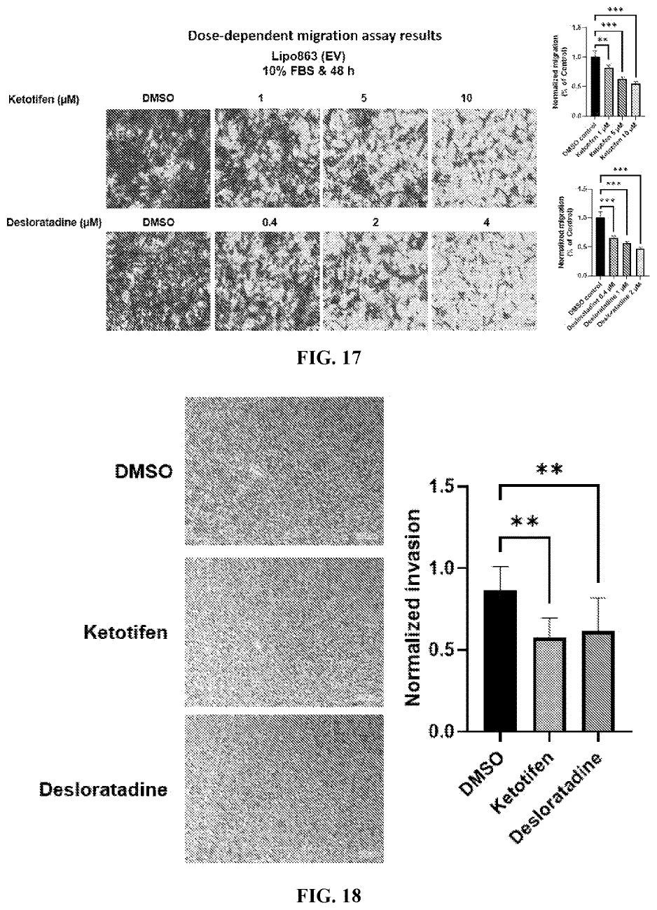

Invasiveness is important parameter used to characterize the aggressive behavior of cancer cells. To test the effect of Ketotifen and Desloratadine on the invasiveness of cells, invasive DDLPS cell line Lipo246 was used. As shown in FIG. 18, after incubating cells for 48 hours at doses of 20 M and 4 M of Ketotifen and Desloratadine, respectively, a significant decrease in invasion ability of Lipo246 cells was observed for both Ketotifen and Desloratadine (30% and 25%, respectively).

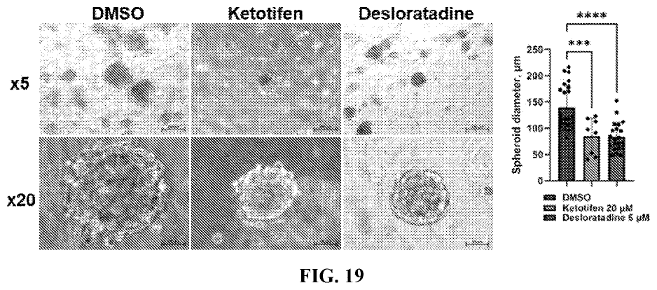

To evaluate the impact of IWS1 inhibition on spheroid formation, a matrigel extracellular matrix scaffold was employed. 14-day treatment with Ketotifen or Desloratadine significantly reduced spheroid formation (both the number and size of spheroids), confirming their impact on tumor growth and cell proliferation. FIG. 19 shows representative microscopic images of spheroids of Lipo863 cells after 14-day treatment with Ketotifen and Desloratadine, ***p<0.001, ****p<0.0001.

Based on the outcome of these experiments, the aforementioned chemical pattern is considered the consensus structure of known active compounds and therefore a hypothetical lead structure. A subset of in-stock chemicals from the Zinc library [5] was generated by substructure search, and a follow-up virtual screening was performed to discover more potent drug candidates and to elucidate possible optimizations based on the known actives. To account for the flexibility and possible alternate conformations of IWS1, a collection of NMR-resolved human IWS1 structures was retrieved from the PDB repository [6][7]. Along with the AlphaFold structure, the ensemble of experimental and predicted IWS1 structures serve as the receptors in molecular docking. The sum of the potencies against all receptors is considered the major descriptor for compound selection. The follow-up virtual screening provides theoretical evidence that many other compounds containing hypothetical lead structures may be potent inhibitors for IWS1. Furthermore, the selection of compounds reveals two potential directions of optimization: (i) extended hydrophobic areas by peri-fused ring systems; and (ii) hydrophilic chains as add-ons to the above-mentioned tricyclic ring patterns.

Due to the largely unstructured nature of IWS1 and the absence of an established high-throughput, experimental screening technique, IWS1 has never been purposed as a drug target, and no known ligands have been reported for IWS1. The establishment of the above-described class of compounds is, to our knowledge, the first effort to identify inhibitors of IWS1 and to use the inhibitors as treatments for cancer including liposarcoma.

### REFERENCES

[1] Diebold, M. L., M. Koch, E. Loeliger, V. Cura, F. Winston, J. Cavarelli and C. Romier (2010). “The structure of an Iwsl/Spt6 complex reveals an interaction domain conserved in TFIIS, Elongin A and Med26.” EMBO J 29(23): 3979-3991.
[2] Eberhardt, J., D. Santos-Martins, A. F. Tillack and S. Forli (2021). “AutoDock Vina 1.2.0: New Docking Methods, Expanded Force Field, and Python Bindings.” J Chem Inf Model 61(8): 3891-3898.
[3]^{a}Irwin, J. J., K. G. Tang, J. Young, C. Dandarchuluun, B. R. Wong, M. Khurelbaatar, Y. S. Moroz, J. Mayfield and R. A. Sayle (2020). “ZINC20-A Free Ultralarge-Scale Chemical Database for Ligand Discovery.” J Chem Inf Model 60(12): 6065-6073.
^{b}Jumper, J., R. Evans, A. Pritzel, T. Green, M. Figurnov, O. Ronneberger, K. Tunyasuvunakool, R. Bates, A. Zidek, A. Potapenko, A. Bridgland, C. Meyer, S. A. A. Kohl, A. J. Ballard, A. Cowie, B. Romera-Paredes, S. Nikolov, R. Jain, J. Adler, T. Back, S. Petersen, D. Reiman, E. Clancy, M. Zielinski, M. Steinegger, M. Pacholska, T. Berghammer, S. Bodenstein, D. Silver, O. Vinyals, A. W. Senior, K. Kavukcuoglu, P. Kohli and D. Hassabis (2021). “Highly accurate protein structure prediction with AlphaFold.” Nature 596(7873): 583-589.
[4] Varadi, M., S. Anyango, M. Deshpande, S. Nair, C. Natassia, G. Yordanova, D. Yuan, O. Stroe, G. Wood, A. Laydon, A. Zidek, T. Green, K. Tunyasuvunakool, S. Petersen, J. Jumper, E. Clancy, R. Green, A. Vora, M. Lutfi, M. Figurnov, A. Cowie, N. Hobbs, P. Kohli, G. Kleywegt, E. Birney, D. Hassabis and S. Velankar (2022). “AlphaFold Protein Structure Database: massively expanding the structural coverage of protein-sequence space with high-accuracy models.” Nucleic Acids Res 50(D1): D439-D444.
[5] Zhang, L., A. G. Fletcher, V. Cheung, F. Winston and L. A. Stargell (2008). “SpnI regulates the recruitment of Spt6 and the Swi/Snf complex during transcriptional activation by RNA polymerase II.” Mol Cell Biol 28(4): 1393-1403.
[6] Berman, H. M., J. Westbrook, Z. Feng, G. Gilliland, T. N. Bhat, H. Weissig, I. N. Shindyalov and P. E. Bourne (2000). “The Protein Data Bank.” Nucleic Acids Research 28(1): 235-242.
[7] Cermakova, K., J. Demeulemeester, V. Lux, M. Nedomova, S. R. Goldman, E. A. Smith, P. Srb, R. Hexnerova, M. Fabry, M. Madlikova, M. Horejsi, J. De Rijck, Z. Debyser, K. Adelman, H. C. Hodges and V. Veverka (2021). “A ubiquitous disordered protein interaction module orchestrates transcription elongation.” Science 374(6571): 1113-1121.
[8] Beane, J., Y. Wang, H. Zhang, A. L. Ferlita, S. P. Nipin, M. Goryunova, P. Sarchet, Z. Hu, M. Sorkin, A. Kim, H. Huang, H. Zhu, A. Tsung and R. Pollock. “Phosphorylation of IWS1 by AKT Maintains Liposarcoma Tumor Heterogeneity through Preservation of Cancer Stem Cell Phenotypes and Mesenchymal-Epithelial Plasticity.” Oncogenesis. [In press]

The compounds, compositions, and methods of the appended claims are not limited in scope by the specific compounds, compositions, and methods described herein, which are intended as illustrations of a few aspects of the claims. Any compounds, compositions, and methods that are functionally equivalent are intended to fall within the scope of the claims. Various modifications of the compounds, compositions, and methods in addition to those shown and described herein are intended to fall within the scope of the appended claims. Further, while only certain representative compounds, components, compositions, and method steps disclosed herein are specifically described, other combinations of the compounds, components, compositions, and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than where noted, all numbers expressing geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

Unless defined otherwise, all technical and scientific terms used herein have the same meanings as commonly understood by one of skill in the art to which the disclosed invention belongs. Publications cited herein and the materials for which they are cited are specifically incorporated by reference.

What is claimed is:

1. A method for treating cancer in a subject in need thereof, the method comprising administering to the subject an effective amount of an IWS1 inhibitor to treat the cancer.

2. The method of claim 1, wherein the cancer comprises a solid tumor.

3. The method of any one of claims 1-2, wherein the cancer comprises a soft tissue sarcoma.

4. The method of any one of claims 1-3, wherein the cancer is characterized by elevated levels of IWS1.

5. The method of any one of claims 1-4, wherein the cancer comprises a liposarcoma (LPS).

6. The method of any one of claims 1-5, wherein the cancer comprises dedifferentiated LPS (ddLPS).

7. The method of any one of claims 1-6, wherein the IWS1 inhibitor is administered in combination with an additional treatment for the cancer.

8. The method of claim 7, wherein the additional treatment for the cancer comprises radiotherapy, surgery, administration of one or more additional active agents, or a combination thereof.

9. The method of any one of claims 1-8, wherein the IWS1 inhibitor reduces the migration ability of cancer cells in the subject.

10. The method of any one of claims 1-9, wherein the IWS1 inhibitor reduces the invasiveness of cancer cells in the subject.

11. The method of any one of claims 1-10, wherein the IWS1 inhibitor reduces metastasis of the cancer.

12. A method for preventing metastisis of a liposarcoma in a subject or reducing the rate of metastisis of a liposarcoma, the method comprising administering to the subject an effective amount of an IWS1 inhibitor to prevent metastisis of the liposarcoma or reduce the rate of metastisis of the liposarcoma.

13. The method of claim 12, wherein the liposarcoma comprises dedifferentiated LPS (ddLPS).

14. A method for reducing the migration of liposarcoma cells in a subject, the method comprising administering to the subject an effective amount of an IWS1 inhibitor to reduce the migration of the liposarcoma cells.

15. The method of claim 14, wherein the liposarcoma cells comprise dedifferentiated LPS (ddLPS) cells.

16. A method for reducing the invasiveness of liposarcoma cells in a subject, the method comprising administering to the subject an effective amount of an IWS1 inhibitor to reduce the invasiveness of the liposarcoma cells.

17. The method of claim 16, wherein the liposarcoma cells comprise dedifferentiated LPS (ddLPS) cells.

18. The method of any one of claims 1-17, wherein the subject is a human.

19. The method of any one of claims 1-18, wherein the IWS1 inhibitor targets an IWS1/Spt6 interface.

20. The method of any one of claims 1-19, wherein the IWS1 inhibitor reduces the binding of IWS1 and Spt6.

21. The method of any one of claims 1-20, wherein the IWS1 inhibitor reduces the formation of the RNAPII complex.

22. The method of any one of claims 1-21, wherein the IWS1 inhibitor down-regulates the transcriptional activities of RNAPII.

23. The method of any one of claims 1-22, wherein the IWS1 inhibitor comprises a tricyclic fused ring system.

24. The method of any one of claims 1-23, wherein the IWS1 inhibitor is defined by Formula I or Formula II 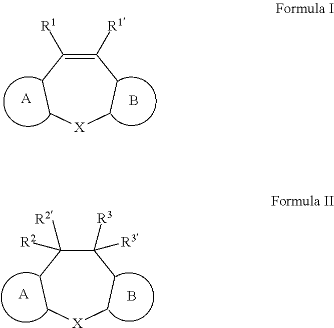
or a pharmaceutically acceptable salt or prodrug thereof, wherein
X is NR⁴, CR⁵R^{5′}, or —C═CR⁶R^{6′};
A and B are each individually selected from a fused 3-10 membered monocyclic or bicyclic cycloalkyl ring, a fused 3-10 membered monocyclic or bicyclic hecycloalkyl ring, a fused 5-11 membered monocyclic or bicyclic aryl ring, and a fused 5-11 membered monocyclic or bicyclic heteroaryl ring, each optionally substituted by 1, 2, 3, or 4 independently selected R^{A} groups;
R¹ and R^{1′} are individually selected from H, halo, OH, OR^{B}, CN, NO₂, C₁₋₄ alkyl, C₁₋₄ haloalkyl, C₂₋₄ alkenyl, or C₂₋₄ alkynyl, wherein the C₁₋₄ alkyl, C₁₋₄ haloalkyl, C₂₋₄ alkenyl, and C₂₋₄ alkynyl groups are each optionally substituted by 1, 2, 3, or 4 independently selected R^{A} groups;
R² and R^{2′} are individually selected from H, halo, OH, OR^{B}, CN, NO₂, C₁₋₄ alkyl, C₁₋₄ haloalkyl, C₂₋₄ alkenyl, or C₂₋₄ alkynyl, wherein the C₁₋₄ alkyl, C₁₋₄ haloalkyl, C₂₋₄ alkenyl, and C₂₋₄ alkynyl groups are each optionally substituted by 1, 2, 3, or 4 independently selected R^{A} groups; or wherein R² and R^{2′} together form a ketone group;
R³ and R^{3′} are individually selected from H, halo, OH, OR^{B}, CN, NO₂, C₁₋₄ alkyl, C₁₋₄ haloalkyl, C₂₋₄ alkenyl, or C₂₋₄ alkynyl, wherein the C₁₋₄ alkyl, C₁₋₄ haloalkyl, C₂₋₄ alkenyl, and C₂₋₄ alkynyl groups are each optionally substituted by 1, 2, 3, or 4 independently selected R^{A} groups; or wherein R³ and R^{3′} together form a ketone group;
R⁴ is selected from hydrogen, C₁₋₆ alkyl, C₂₋₆ alkenyl, C₂₋₆ alkynyl, C₁₋₄ haloalkyl, C₁₋₆ alkoxy, C₁₋₆ haloalkoxy, a 3-10 membered monocyclic or bicyclic cycloalkyl ring, a 3-10 membered monocyclic or bicyclic hecycloalkyl ring, a 5-11 membered monocyclic or bicyclic aryl ring, a 5-11 membered monocyclic or bicyclic heteroaryl ring, cyano-C₁₋₃ alkyl, HO—C₁₋₃ alkyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino, each optionally substituted by 1, 2, 3, or 4 independently selected R^{A} groups;
R⁵ and R^{5′} are individually selected from H, halo, OH, OR^{B}, CN, NO₂, C₁₋₆ alkyl, C₂₋₆ alkenyl, C₂₋₆ alkynyl, C₁₋₄ haloalkyl, C₁₋₆ alkoxy, C₁₋₆ haloalkoxy, a 3-10 membered monocyclic or bicyclic cycloalkyl ring, a 3-10 membered monocyclic or bicyclic hecycloalkyl ring, a 5-11 membered monocyclic or bicyclic aryl ring, a 5-11 membered monocyclic or bicyclic heteroaryl ring, each optionally substituted by 1, 2, 3, or 4 independently selected R^{A} groups; or wherein R⁵ and R^{5′} together form a ketone group;
R⁶ and R^{6′} are individually selected from H, halo, OH, OR^{B}, CN, NO₂, C₁₋₆ alkyl, C₂₋₆ alkenyl, C₂₋₆ alkynyl, C₁₋₄ haloalkyl, C₁₋₆ alkoxy, C₁₋₆ haloalkoxy, a 3-10 membered monocyclic or bicyclic cycloalkyl ring, a 3-10 membered monocyclic or bicyclic hecycloalkyl ring, a 5-11 membered monocyclic or bicyclic aryl ring, a 5-11 membered monocyclic or bicyclic heteroaryl ring, each optionally substituted by 1, 2, 3, or 4 independently selected R^{A} groups;
each R^{A} is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino; and
R^{B} is selected from hydrogen, C₁₋₆ alkyl, C₁₋₄haloalkyl, C₂₋₆ alkenyl, C₂₋₆ alkynyl, C₃₋₁₀ cycloalkyl, and 6-10 membered aryl, wherein the C₁₋₆ alkyl, C₁₋₄haloalkyl, C₂₋₆ alkenyl, C₂₋₆ alkynyl, C₃₋₁₀ cycloalkyl, and 6-10 membered aryl are each optionally substituted by 1, 2, 3, or 4 independently selected R^{A} groups.

25. The method of any one of claims 1-24, wherein the IWS1 inhibitor is defined by Formula IA or Formula IIA 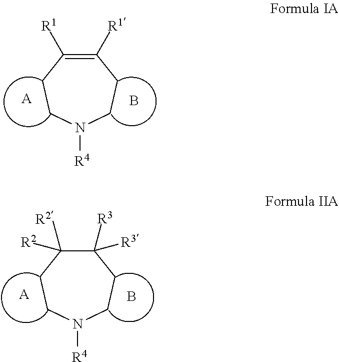
or a pharmaceutically acceptable salt or prodrug thereof, wherein
A and B are each individually selected from a fused 3-10 membered monocyclic or bicyclic cycloalkyl ring, a fused 3-10 membered monocyclic or bicyclic hecycloalkyl ring, a fused 5-11 membered monocyclic or bicyclic aryl ring, and a fused 5-11 membered monocyclic or bicyclic heteroaryl ring, each optionally substituted by 1, 2, 3, or 4 independently selected R^{A} groups;
R¹ and R^{1′} are individually selected from H, halo, OH, OR^{B}, CN, NO₂, C₁₋₄ alkyl, C₁₋₄ haloalkyl, C₂₋₄ alkenyl, or C₂₋₄ alkynyl, wherein the C₁₋₄ alkyl, C₁₋₄ haloalkyl, C₂₋₄ alkenyl, and C₂₋₄ alkynyl groups are each optionally substituted by 1, 2, 3, or 4 independently selected R^{A} groups;
R² and R^{2′} are individually selected from H, halo, OH, OR^{B}, CN, NO₂, C₁₋₄ alkyl, C₁₋₄ haloalkyl, C₂₋₄ alkenyl, or C₂₋₄ alkynyl, wherein the C₁₋₄ alkyl, C₁₋₄ haloalkyl, C₂₋₄ alkenyl, and C₂₋₄ alkynyl groups are each optionally substituted by 1, 2, 3, or 4 independently selected R^{A} groups; or wherein R² and R^{2′} together form a ketone group;
R³ and R^{3′} are individually selected from H, halo, OH, OR^{B}, CN, NO₂, C₁₋₄ alkyl, C₁₋₄ haloalkyl, C₂₋₄ alkenyl, or C₂₋₄ alkynyl, wherein the C₁₋₄ alkyl, C₁₋₄ haloalkyl, C₂₋₄ alkenyl, and C₂₋₄ alkynyl groups are each optionally substituted by 1, 2, 3, or 4 independently selected R^{A} groups; or wherein R³ and R^{3′} together form a ketone group;
R⁴ is selected from hydrogen, C₁₋₆ alkyl, C₂₋₆ alkenyl, C₂₋₆ alkynyl, C₁₋₄ haloalkyl, C₁₋₆ alkoxy, C₁₋₆ haloalkoxy, a 3-10 membered monocyclic or bicyclic cycloalkyl ring, a 3-10 membered monocyclic or bicyclic hecycloalkyl ring, a 5-11 membered monocyclic or bicyclic aryl ring, a 5-11 membered monocyclic or bicyclic heteroaryl ring, cyano-C₁₋₃ alkyl, HO—C₁₋₃ alkyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino, each optionally substituted by 1, 2, 3, or 4 independently selected R^{A} groups;
each R^{A} is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino; and
R^{B} is selected from hydrogen, C₁₋₆ alkyl, C₁₋₄haloalkyl, C₂₋₆ alkenyl, C₂₋₆ alkynyl, C₃₋₁₀ cycloalkyl, and 6-10 membered aryl, wherein the C₁₋₆ alkyl, C₁₋₄haloalkyl, C₂₋₆ alkenyl, C₂₋₆ alkynyl, C₃₋₁₀ cycloalkyl, and 6-10 membered aryl are each optionally substituted by 1, 2, 3, or 4 independently selected R^{A} groups.

26. The method of claim 25, wherein R⁴ is C₁₋₆ alkyl optionally substituted by 1, 2, 3, or 4 independently selected R^{A} groups.

27. The method of any one of claims 1-24, wherein the IWS1 inhibitor is defined by Formula IB or Formula IIB 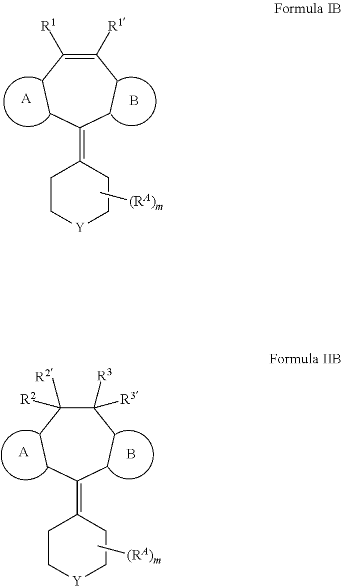
or a pharmaceutically acceptable salt or prodrug thereof, wherein
Y is NR⁴ or CR⁵R^{5′};
A and B are each individually selected from a fused 3-10 membered monocyclic or bicyclic cycloalkyl ring, a fused 3-10 membered monocyclic or bicyclic hecycloalkyl ring, a fused 5-11 membered monocyclic or bicyclic aryl ring, and a fused 5-11 membered monocyclic or bicyclic heteroaryl ring, each optionally substituted by 1, 2, 3, or 4 independently selected R^{A} groups;
R¹ and R^{1′} are individually selected from H, halo, OH, ORB, CN, NO₂, C₁₋₄ alkyl, C₁₋₄ haloalkyl, C₂₋₄ alkenyl, or C₂₋₄ alkynyl, wherein the C₁₋₄ alkyl, C₁₋₄ haloalkyl, C₂₋₄ alkenyl, and C₂₋₄ alkynyl groups are each optionally substituted by 1, 2, 3, or 4 independently selected R^{A} groups;
R² and R^{2′} are individually selected from H, halo, OH, ORB, CN, NO₂, C₁₋₄ alkyl, C₁₋₄ haloalkyl, C₂₋₄ alkenyl, or C₂₋₄ alkynyl, wherein the C₁₋₄ alkyl, C₁₋₄ haloalkyl, C₂₋₄ alkenyl, and C₂₋₄ alkynyl groups are each optionally substituted by 1, 2, 3, or 4 independently selected R^{A} groups; or wherein R² and R^{2′} together form a ketone group;
R³ and R^{3′} are individually selected from H, halo, OH, ORB, CN, NO₂, C₁₋₄ alkyl, C₁₋₄ haloalkyl, C₂₋₄ alkenyl, or C₂₋₄ alkynyl, wherein the C₁₋₄ alkyl, C₁₋₄ haloalkyl, C₂₋₄ alkenyl, and C₂₋₄ alkynyl groups are each optionally substituted by 1, 2, 3, or 4 independently selected R^{A} groups; or wherein R³ and R^{3′} together form a ketone group;
R⁴ is selected from hydrogen, C₁₋₆ alkyl, C₂₋₆ alkenyl, C₂₋₆ alkynyl, C₁₋₄ haloalkyl, C₁₋₆ alkoxy, C₁₋₆ haloalkoxy, a 3-10 membered monocyclic or bicyclic cycloalkyl ring, a 3-10 membered monocyclic or bicyclic hecycloalkyl ring, a 5-11 membered monocyclic or bicyclic aryl ring, a 5-11 membered monocyclic or bicyclic heteroaryl ring, cyano-C₁₋₃ alkyl, HO—C₁₋₃ alkyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino, each optionally substituted by 1, 2, 3, or 4 independently selected R^{A} groups;
R⁵ and R^{5′} are individually selected from H, halo, OH, ORB, CN, NO₂, C₁₋₆ alkyl, C₂₋₆ alkenyl, C₂₋₆ alkynyl, C₁₋₄ haloalkyl, C₁₋₆ alkoxy, C₁₋₆ haloalkoxy, a 3-10 membered monocyclic or bicyclic cycloalkyl ring, a 3-10 membered monocyclic or bicyclic hecycloalkyl ring, a 5-11 membered monocyclic or bicyclic aryl ring, a 5-11 membered monocyclic or bicyclic heteroaryl ring, each optionally substituted by 1, 2, 3, or 4 independently selected R^{A} groups;
each R^{A} is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino;
m is an integer from 0 to 4; and
R^{B} is selected from hydrogen, C₁₋₆ alkyl, C₁₋₄haloalkyl, C₂₋₆ alkenyl, C₂₋₆ alkynyl, C₃₋₁₀ cycloalkyl, and 6-10 membered aryl, wherein the C₁₋₆ alkyl, C₁₋₄haloalkyl, C₂₋₆ alkenyl, C₂₋₆ alkynyl, C₃₋₁₀ cycloalkyl, and 6-10 membered aryl are each optionally substituted by 1, 2, 3, or 4 independently selected R^{A} groups.

28. The method of any one of claims 24-27, wherein A is a phenyl ring.

29. The method of any one of claims 24-27, wherein A is a pyridyl ring.

30. The method of any one of claims 24-27, wherein A is a 5-membered heterocyclic ring chosen from a thiophene ring, a pyrrolidine ring, a pyrroline ring, a pyrrole ring, a pyrazolidine ring, an imidazolidine ring, a pyrazoline ring, and imidazoline ring, a pyrazole ring, a imidazole ring, a triazole ring, a furyl ring, a dioxolane ring, a thiophenyl ring, an oxazole ring, an isoxazole ring, an isothiazole ring, a thiazole ring, an oxathiolane ring, oxadiazole ring, or a thiadiazole ring.

31. The method of any one of claims 24-30, wherein B is a phenyl ring.

32. The method of any one of claims 24-30, wherein B is a pyridyl ring.

33. The method of any one of claims 24-30, wherein B is a 5-membered heterocyclic ring chosen from a thiophene ring, a pyrrolidine ring, a pyrroline ring, a pyrrole ring, a pyrazolidine ring, an imidazolidine ring, a pyrazoline ring, and imidazoline ring, a pyrazole ring, a imidazole ring, a triazole ring, a furyl ring, a dioxolane ring, a thiophenyl ring, an oxazole ring, an isoxazole ring, an isothiazole ring, a thiazole ring, an oxathiolane ring, oxadiazole ring, or a thiadiazole ring.

34. The method of any one of claims 27-33, wherein Y is NR⁴.

35. The method of any one of claims 27-34, wherein R⁴ is selected from hydrogen and C₁₋₆ alkyl.

36. The method of any one of claims 27-35, wherein m is 0.

37. The method of any one of claims 1-23, wherein the IWS1 inhibitor comprises a compound shown below

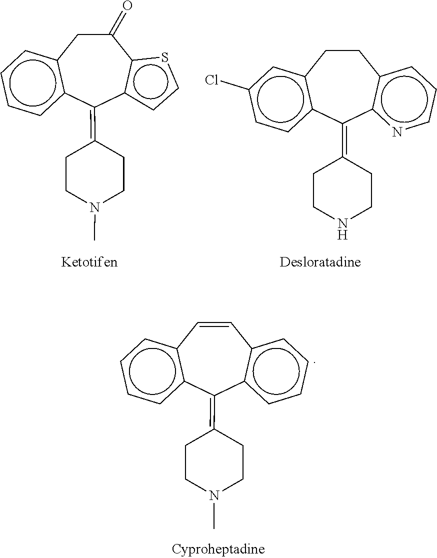

38. The method of any one of claims 1-23, wherein the IWS1 inhibitor comprises a compound shown below 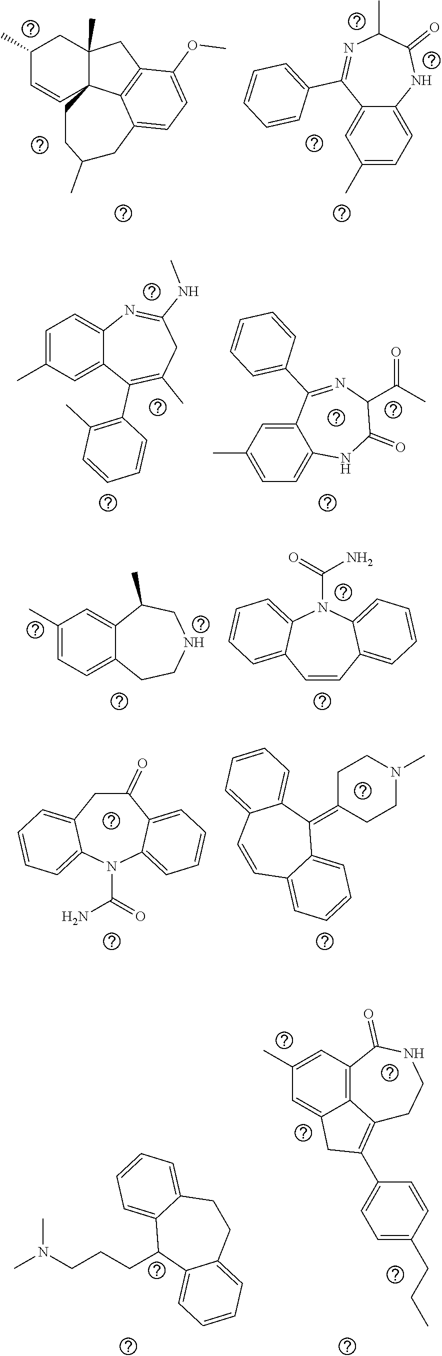 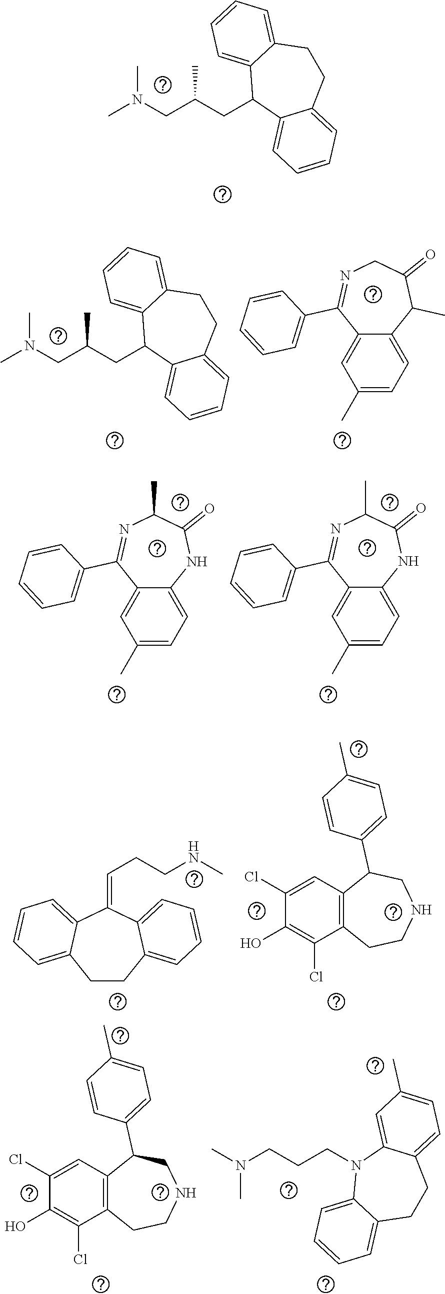 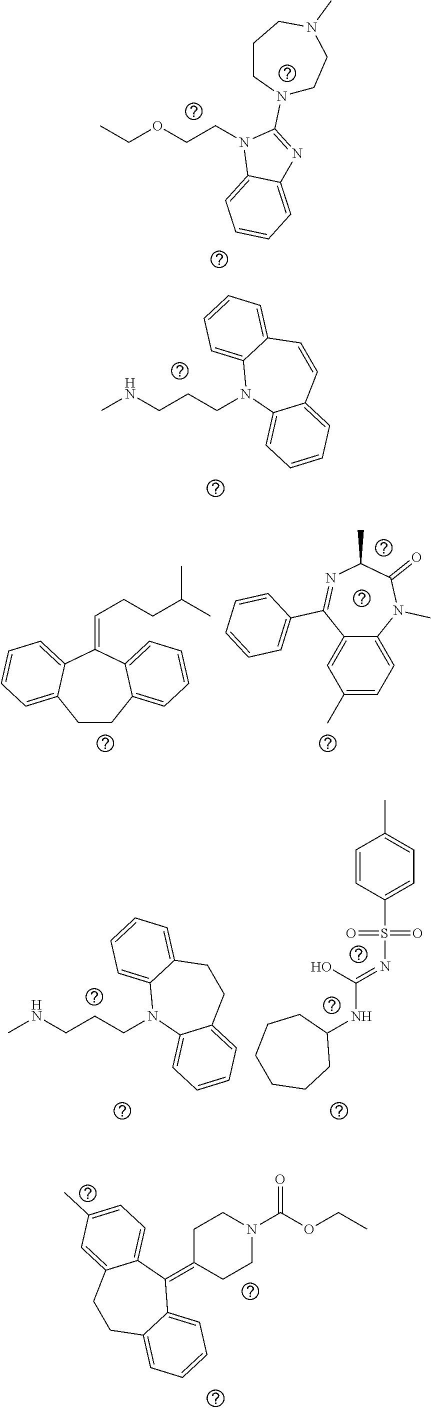 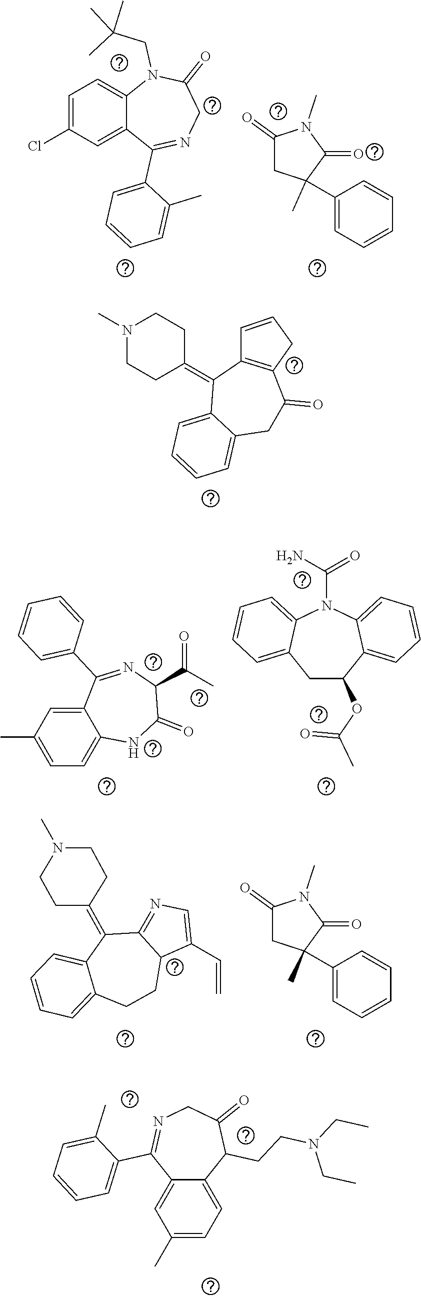 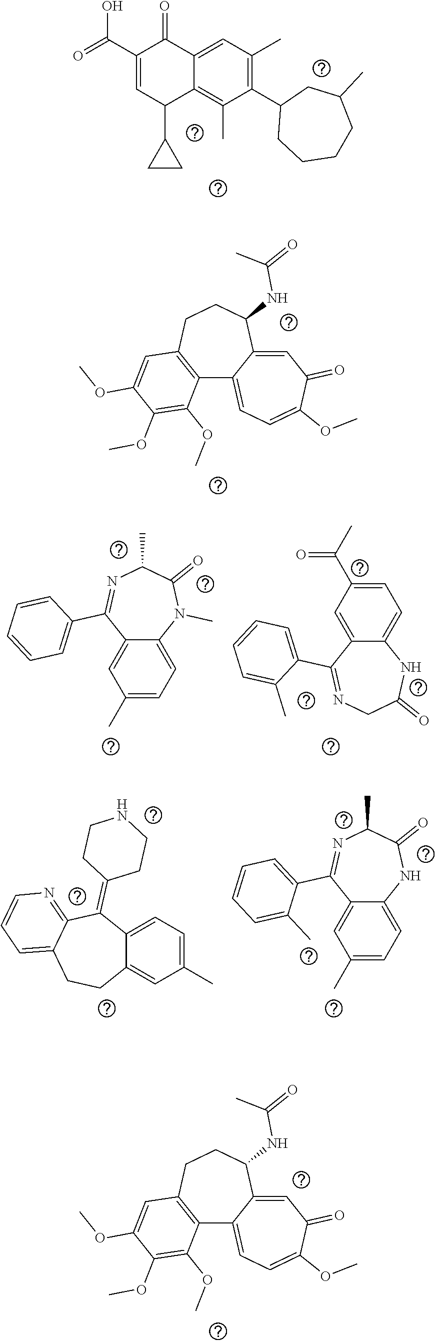 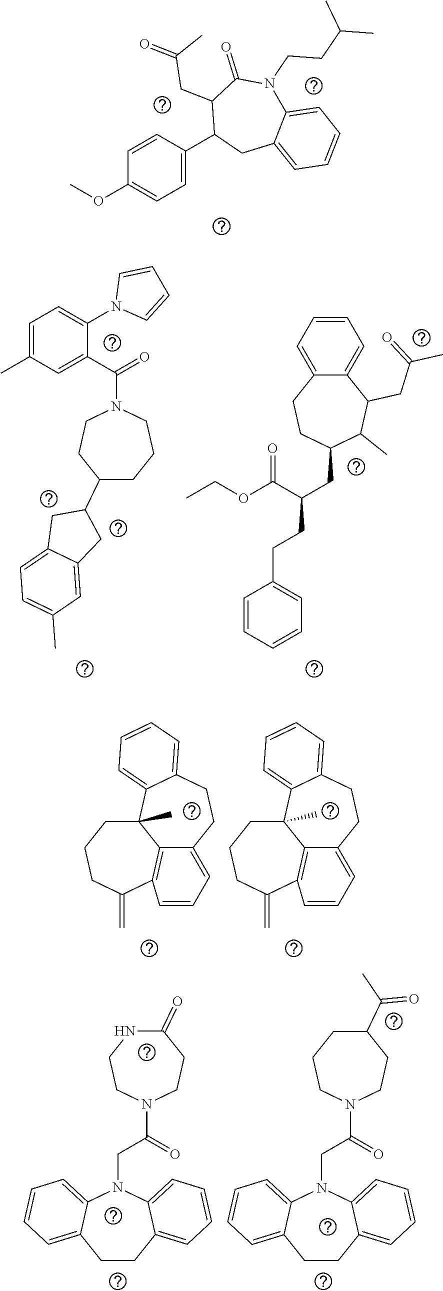 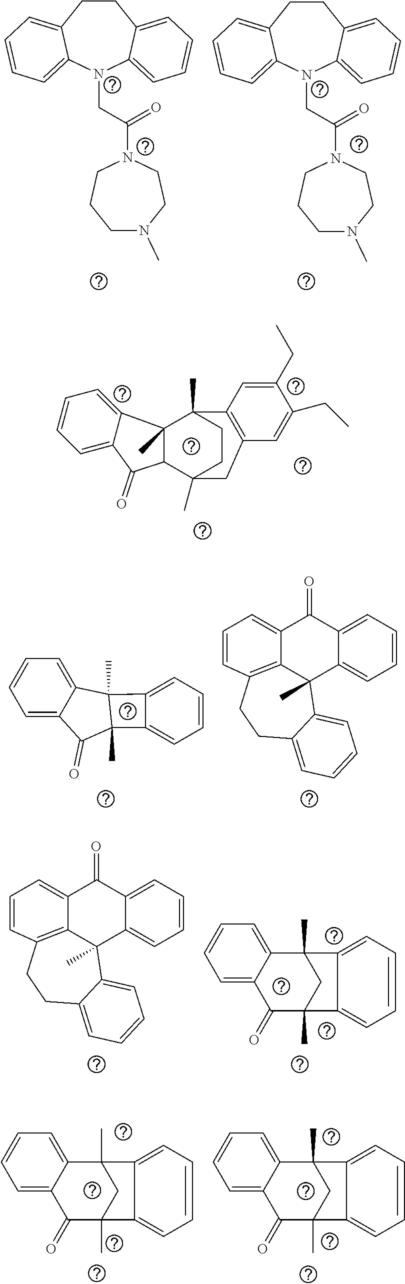 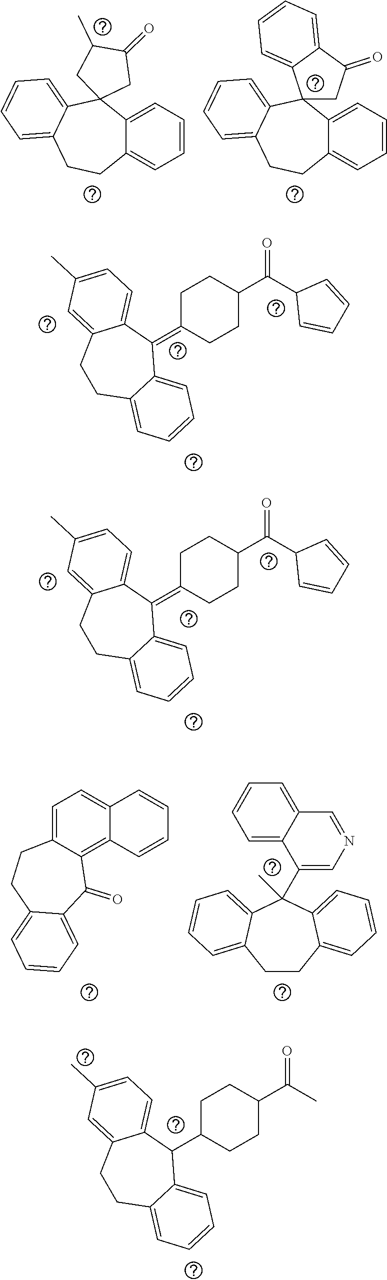 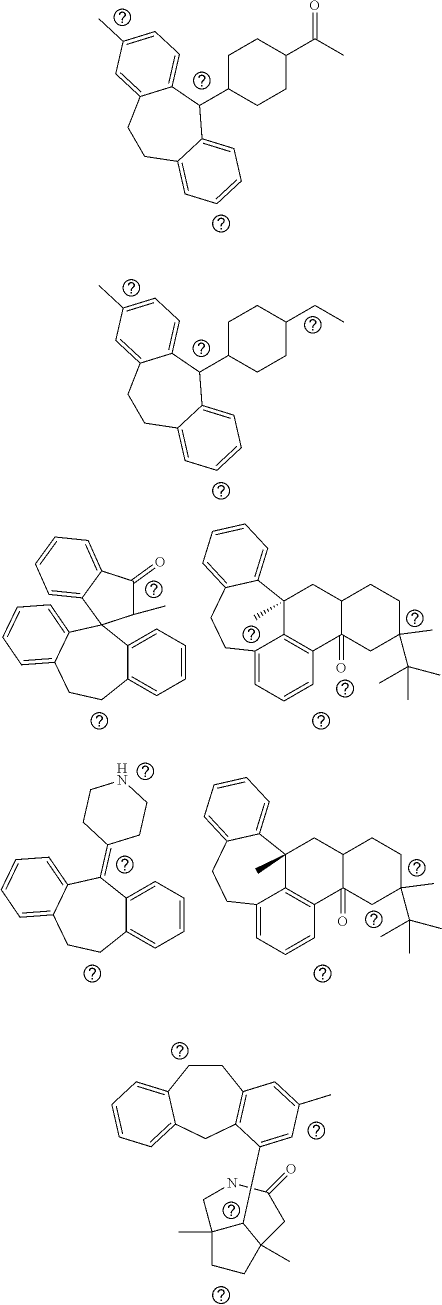 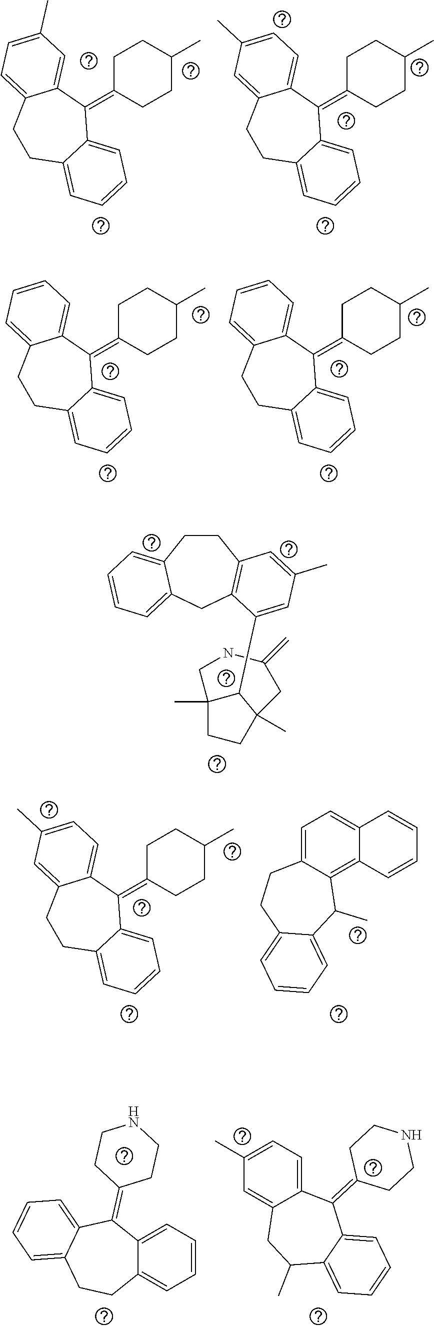 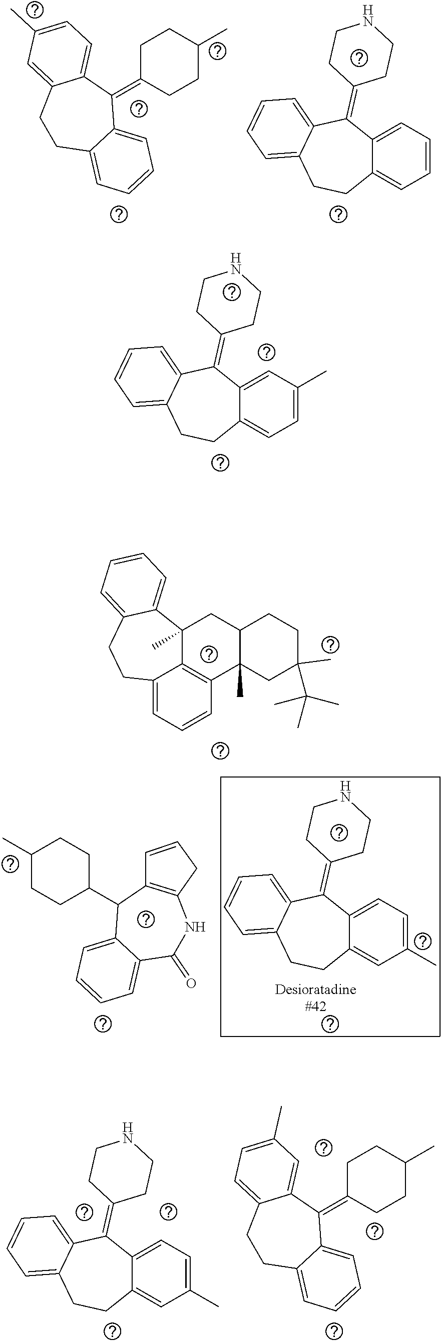 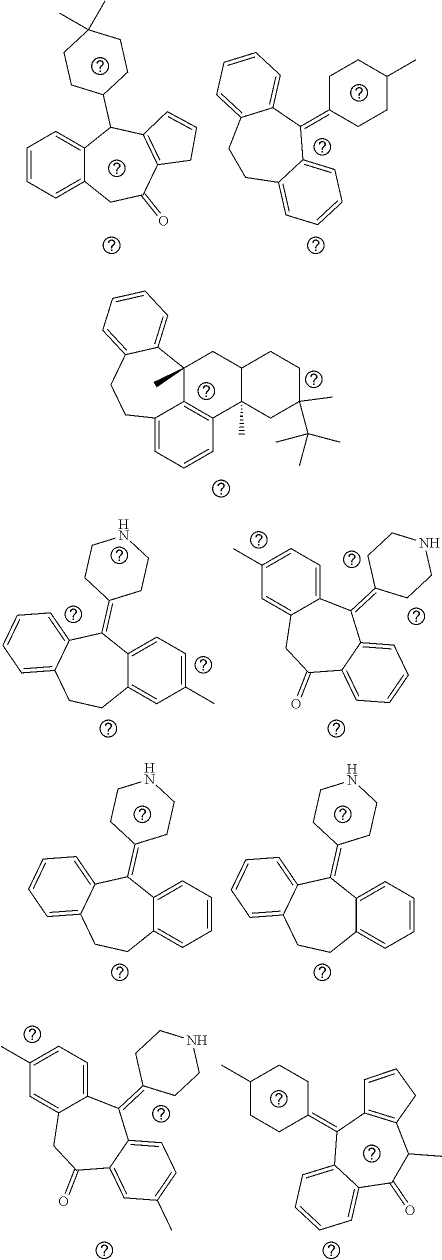 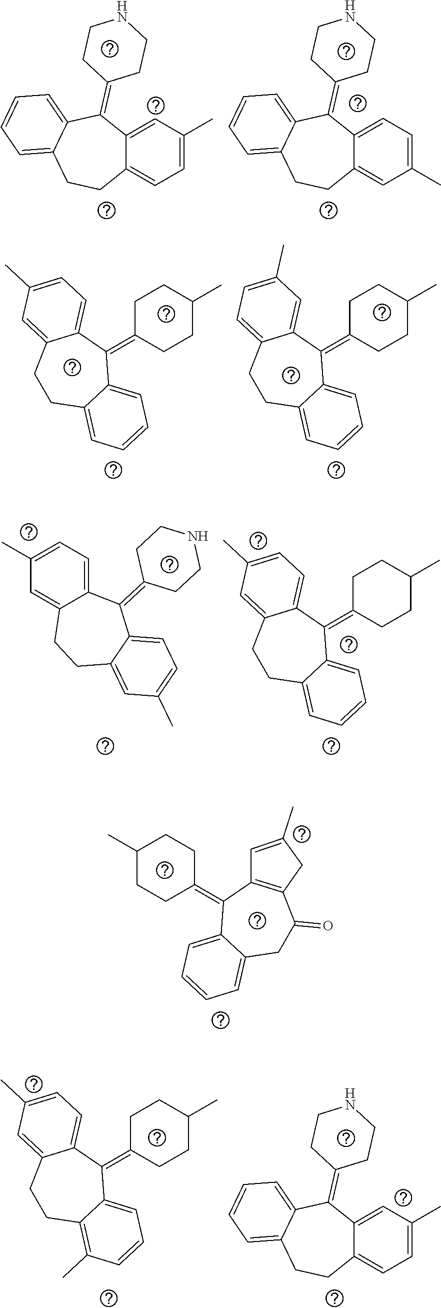 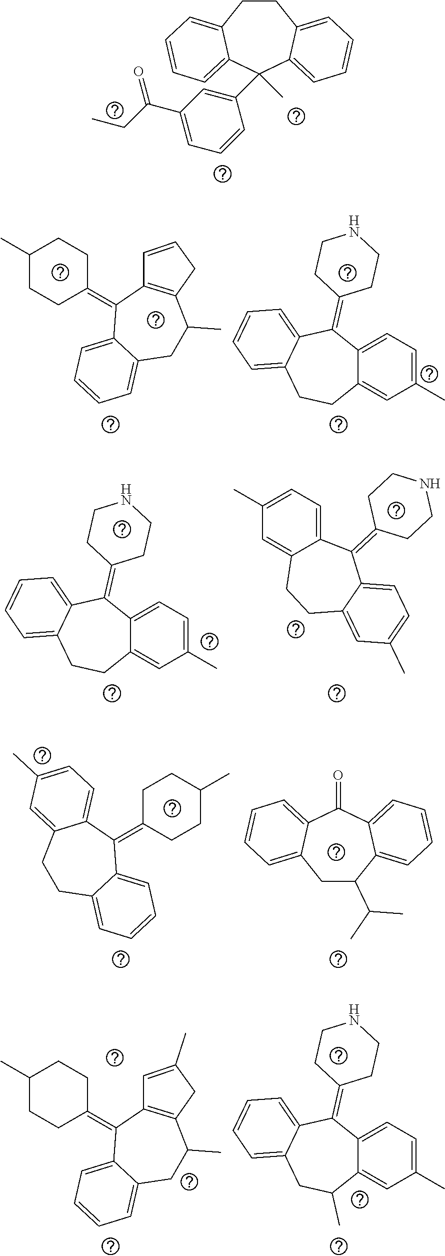 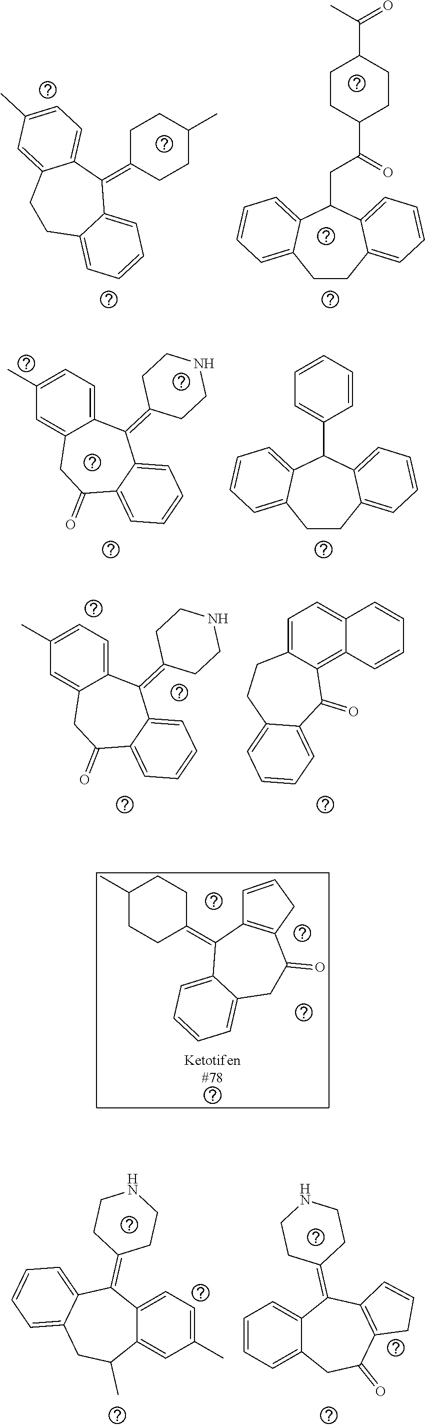 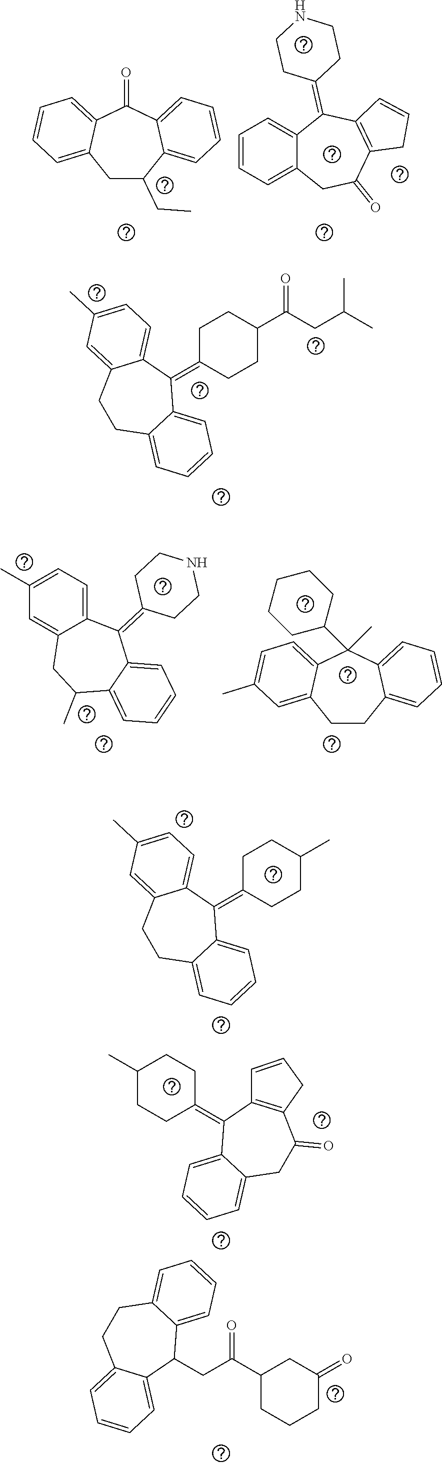 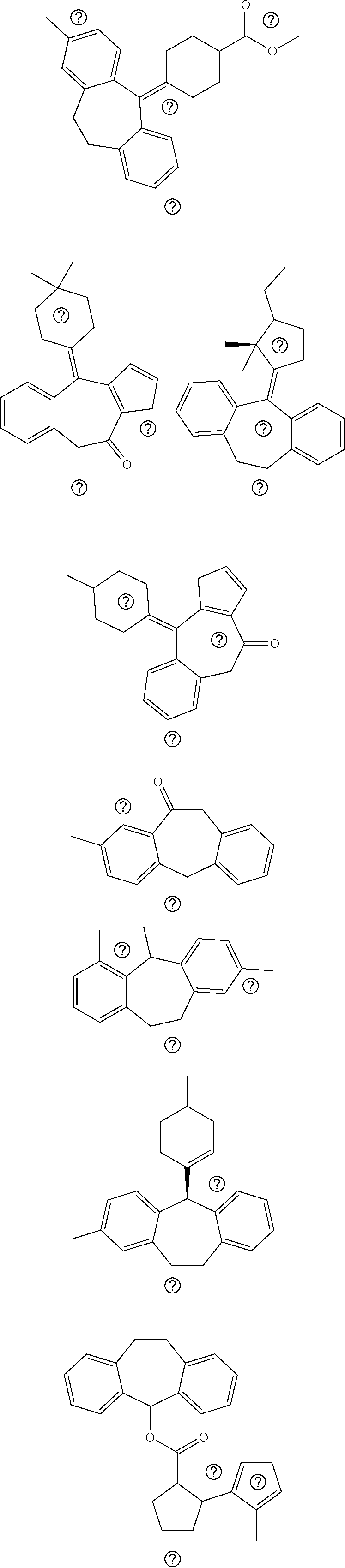 [chemical structure]
